(19) United States
(12) Patent Application Publication
(10) Pub. No.: US 2006/0057643 A1
(43) Pub. Date: Mar. 16, 2006

(54) Methods and compositions for detecting erythrovirus genotypes

(76) Inventors: Laurence R. McCarthy, Great Falls, VA (US); Lilly I. Kong, Covina, CA (US); Xin Su, Irvine, CA (US); Tang Shao, Rosemead, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/223,785

(22) Filed: Sep. 9, 2005

Related U.S. Application Data
(60) Provisional application No. 60/609,083, filed on Sep. 10, 2004.

(51) Int. Cl. C12Q 1/68; G01N 33/53

(52) U.S. Cl. 435/7.1; 435/6

(57) ABSTRACT

The invention provides methods and compositions for rapid, sensitive, and highly specific nucleic acid-based (e.g., DNA based) detection of human erythroviruses, such as the B19, V9, and A6 genotype, in a sample. In general, the methods involve detecting a target nucleic acid having a target sequence of a conserved region of the erythrovirus genomes. The invention also features compositions, including primers, probes, and kits, for use in the methods of the invention.

### CROSS-REFERENCE

This application claims the benefit of U.S. Provisional Application No. 60/609,083, filed Sep. 10, 2004, which application is incorporated herein by reference in its entirety.

### FIELD OF THE INVENTION

The invention relates to detection of human erythroviruses, especially the B19, V9, and A6 genotypes.

### BACKGROUND OF THE INVENTION

The erythroviruses (formerly parvoviruses) constitute a family of viruses that have been associated with diseases or conditions in multiple mammals, including dogs and humans. Erythrovirus genotypes B19, V9/D91.1, and A6/Lali are associated with diseases and syndromes in humans. The human erythroviruses are small 22-nm iscoahedral, non-enveloped DNA viruses whose genome includes a linear single-stranded 5.6 kb DNA molecule that encodes two structural proteins, which are designated VP1 and VP2, and a non-structural protein, designated NS-1. The two proteins are encoded in overlapping reading frames from about nucleotides 2444 to 4789 and about 3125 to 4789, respectively. VP2 constitutes 95% of the capsid and the larger VP1 protein only 5% of the capsid. VP1 is required for the mature conformation of the virus. NS1 (77 kDa), is a nonstructural protein and is present only in the nuclear fraction of infected cells and absent from the cytoplasm and intact virions in sera.

Several diseases and syndromes have been associated with erythrovirus B19 genotype. One disease includes ertyhema infectiosum (EI), which is a common in children and is characterized by fever, headache, nausea, and diarrhea. While these symptoms are typically mild, the consequences of erythrovirus infection in some individuals, such as pregnant women, can be more severe. For example, erythrovirus B19 infection during pregnancy can have significant and potentially fatal effects on the fetus.

Immunodiagnostic methods have been used to identify blood, serum, or plasma that is potentially contaminated with erythrovirus B19 genotype. Many methods detect anti-parvovirus antibodies (IgM or IgG) present in an individual's serum or plasma (e.g., see PCT Nos. WO 96/09391 and WO 96/27799). Immunological methods, however, have limitations on detecting recent or current infections because they rely on detecting the body's response to the infectious agent. Because of the rapid rise in viremia following infection, an individual's blood may contain high levels of parvovirus B19 before anti-parvovirus antibodies are detectable, leading to false negative results. Because viremia is often quickly cleared, a person may remain antibody-positive even when infective particles are not present, leading to false positive results. In addition, the immunodiagnostic methods have also not been able to distinguish between the different erythrovirus genotypes, e.g., B19, V9, and A6, in viremic samples.

Accordingly, there remains a need for the development of reliable diagnostic tests to detect erythroviruses with a sensitivity that allows detection of low titres of virus as may occur early in an infection. In addition, there remains a need for a reliable diagnostic test to distinguish between the different erythrovirus genotypes, e.g., B19, V9, and A6, in viremic samples, in order to prevent transmission of the virus through blood and plasma derivatives or by close personal contact. The present invention addresses these needs.

Literature

Literature of interest includes: Shade et al., J. Virol. (1986) 58:921-936; Brown et al., Ann. Rev. Med. (1997) 48: 59-67; Söderlund et al., Lancet 1997; 349:1063-1065; Hokynar et al., Virology 2002; 302:224-228; Kakkola et al., Arch Virol 2004; 149:1095-1106; Hokynar et al., J Clin Microbiol 2004; 42:2013-2019; Brunstein et al., Virology 2000 Sep. 1;274(2):284-91; U.S. Pat. Nos. 6,642,033; 6,413,716; 6,379,885; 6,287,815; 6,204,044, 6,287,815; 6,379,885; 5,688,669; 5,449,608; U.S. Patent Application Nos: 2003/0170612; 2003/0124578; 2002/0119527; and PCT Nos. WO 96/09391; WO 96/27799; WO 96/09391; WO 99/28439.

### SUMMARY OF THE INVENTION

The invention provides methods and compositions for rapid, sensitive, and highly specific nucleic acid-based (e.g., DNA based) detection of human erythroviruses, such as the B19, V9, and A6 genotypes, in a sample. In general, the methods involve detecting a target nucleic acid having a target sequence of a conserved region of the erythrovirus genomes. The invention also features compositions, including primers, probes, and kits, for use in the methods of the invention.

An advantage of the invention is that it provides for detection of human erythrovirus (human EV) while avoiding detection of viruses that are closely related genetically. Thus, the invention decreases the incidence of false negatives.

Another advantage of the invention is that it decreases the incidence of false positive results that can result from detection of the closely genetically related viruses.

Another advantage of the invention is that it provides for the selective detection of different specific of human erythrovirus, such as the B19, V9, and A6 genotypes.

Still another advantage is that the invention encompasses embodiments that require detection of only a relatively short target sequence. This can be particularly advantageous where the assay uses amplification-based technology, such as real-time PCR.

The present invention can be developed into assays or manufactured into kits to be use in reference laboratories or hospitals for the diagnostics of erythrovirus. The assay can also be utilized in the development and clinical trials of therapeutic drugs for treating diseases caused by erythrovirus infection.

These and other advantages will be readily apparent to the ordinarily skilled artisan upon reading the present specification.

### BRIEF DESCRIPTION OF THE DRAWINGS

FIGS. 1A-1N show the alignment of Human Erythrovirus B19 (GenBank Accession No. NC_{—}000883), A6 (GenBank Accession No. AY064475), and V9 (GenBank Accession No. NC_{—}004295), and denotes the Target Regions I-VIII. Each Target Region is demarcated with arrows and the nucleotide range (inclusive) for each Target Region. As shown in the figure, a portion of the 5′ end of Target Region VI overlaps with a portion of the 3′ end of Target Region V. Moreover, a portion of the 3′ end of Target Region VI overlaps with a portion of the 5′ end of Target Region VII.

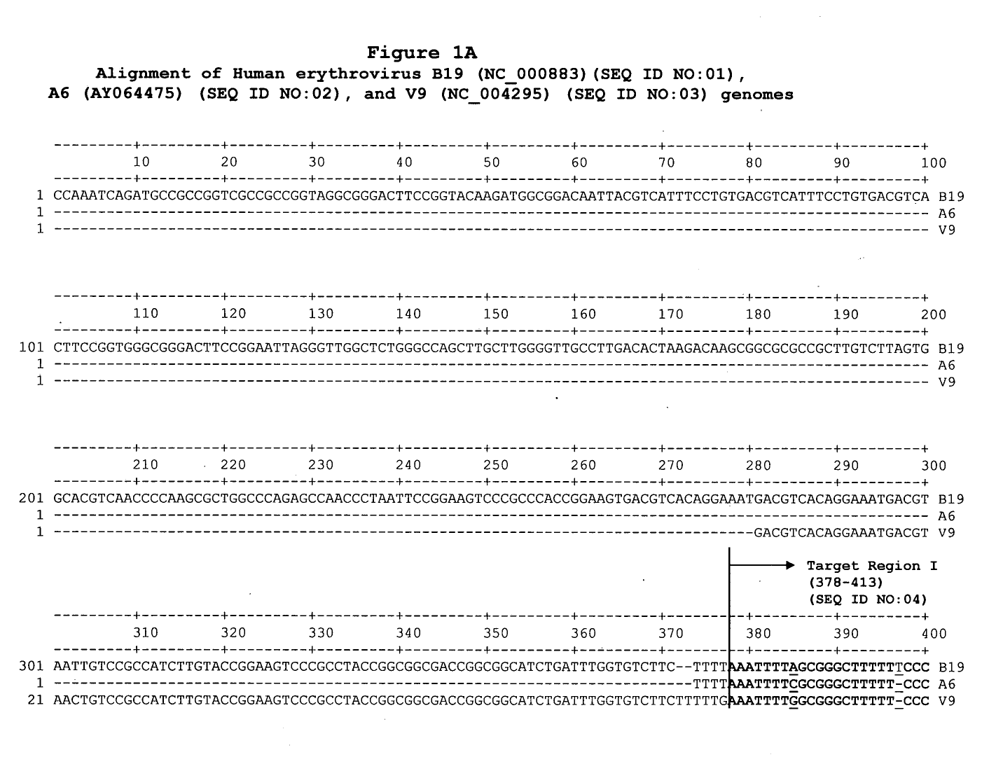

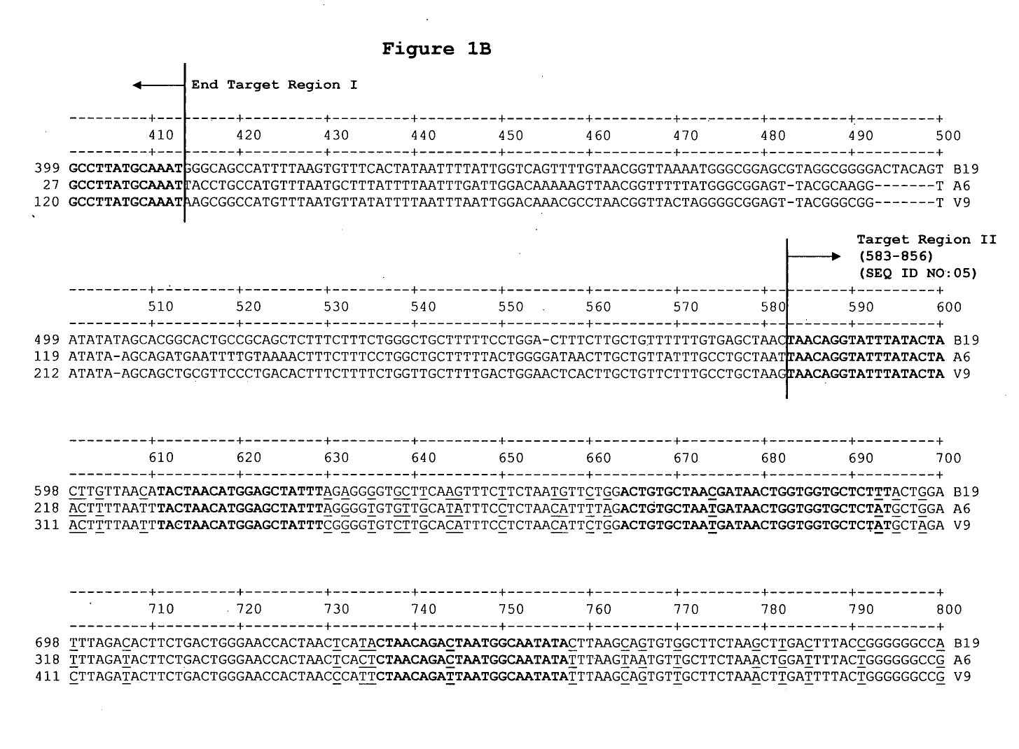

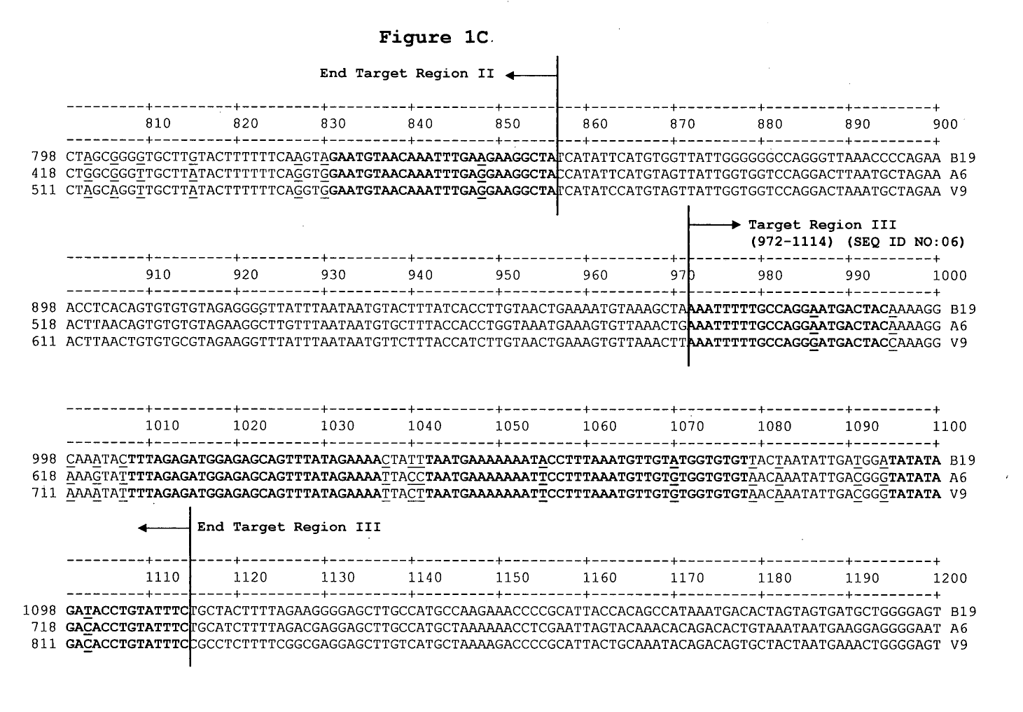

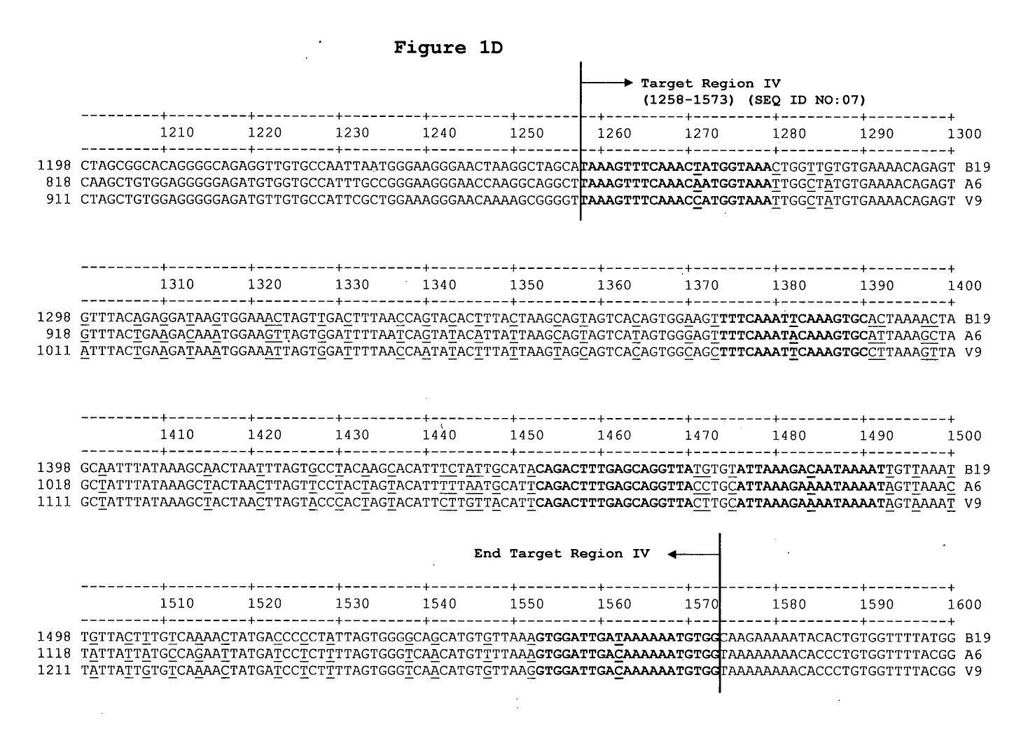

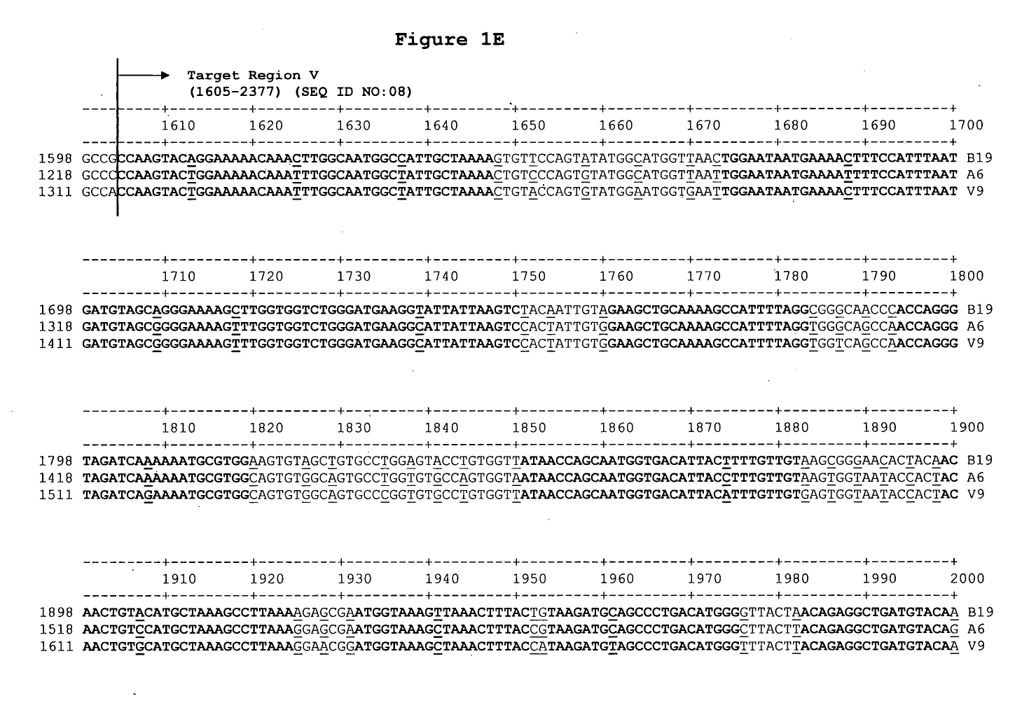

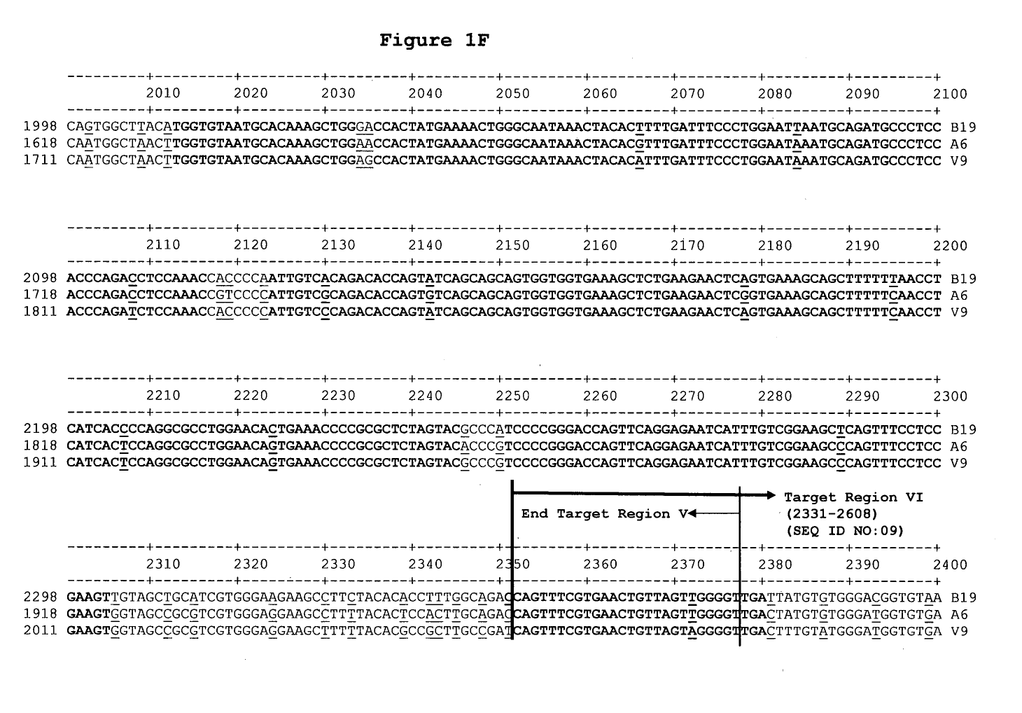

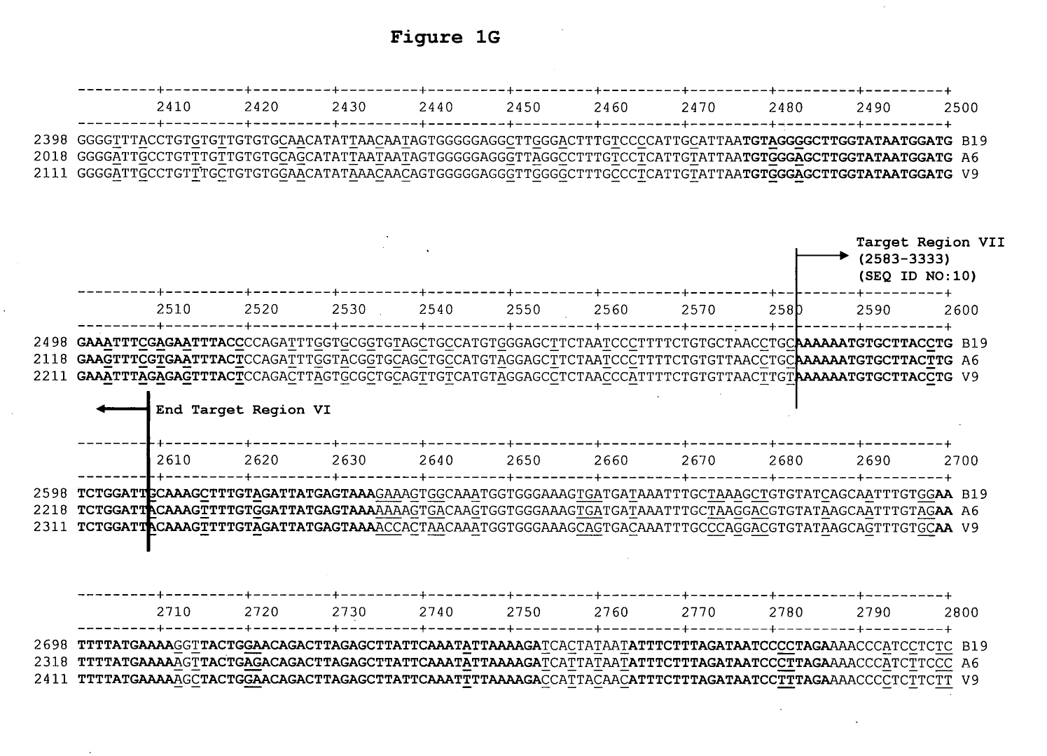

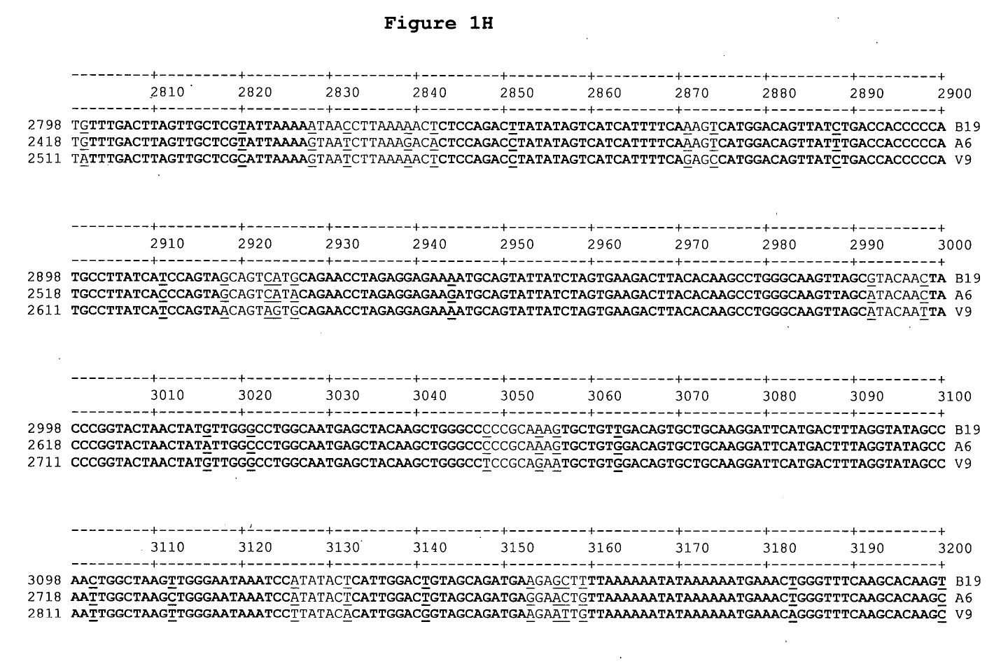

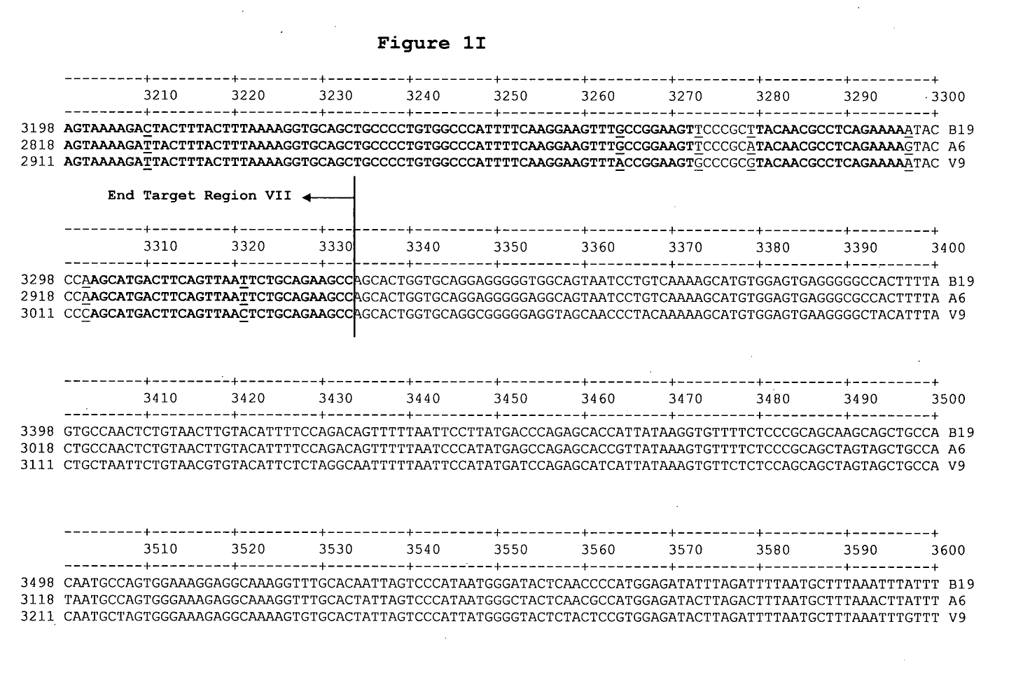

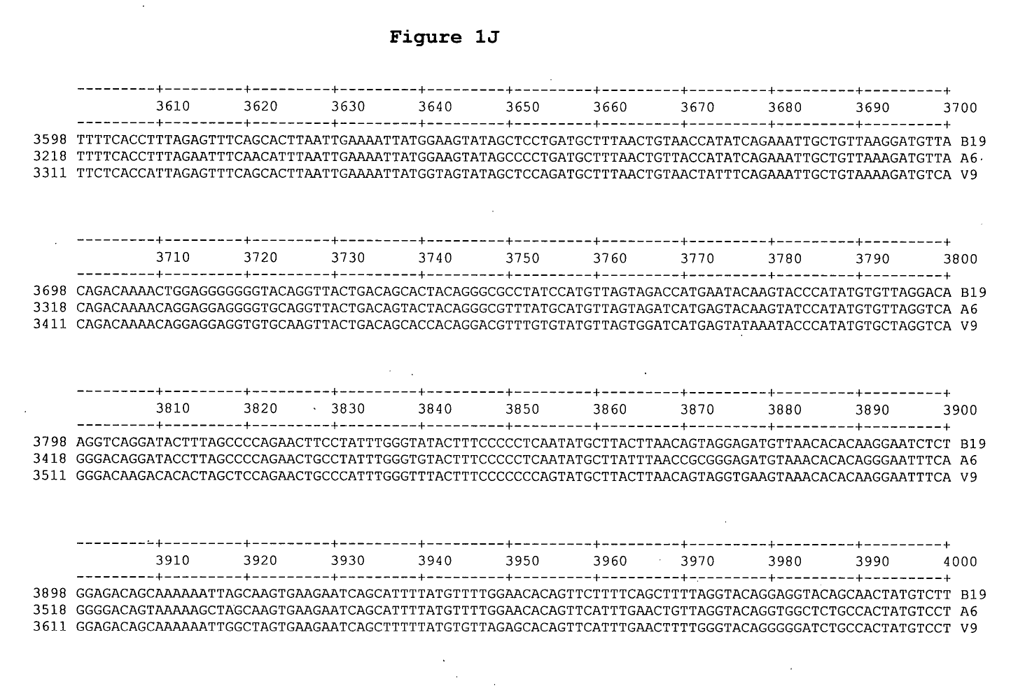

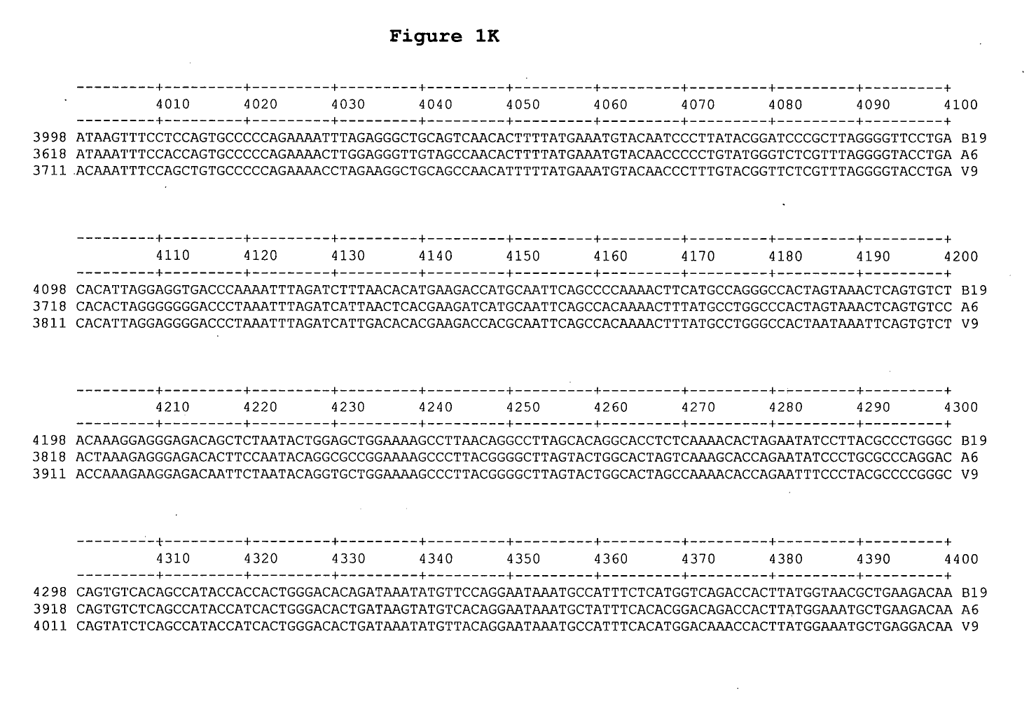

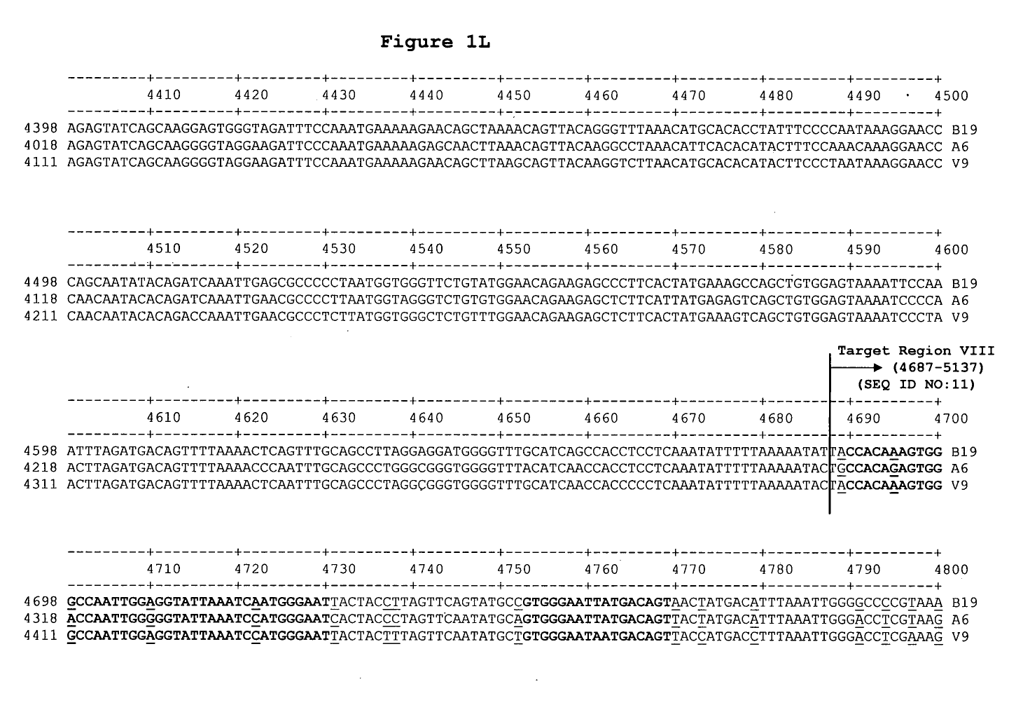

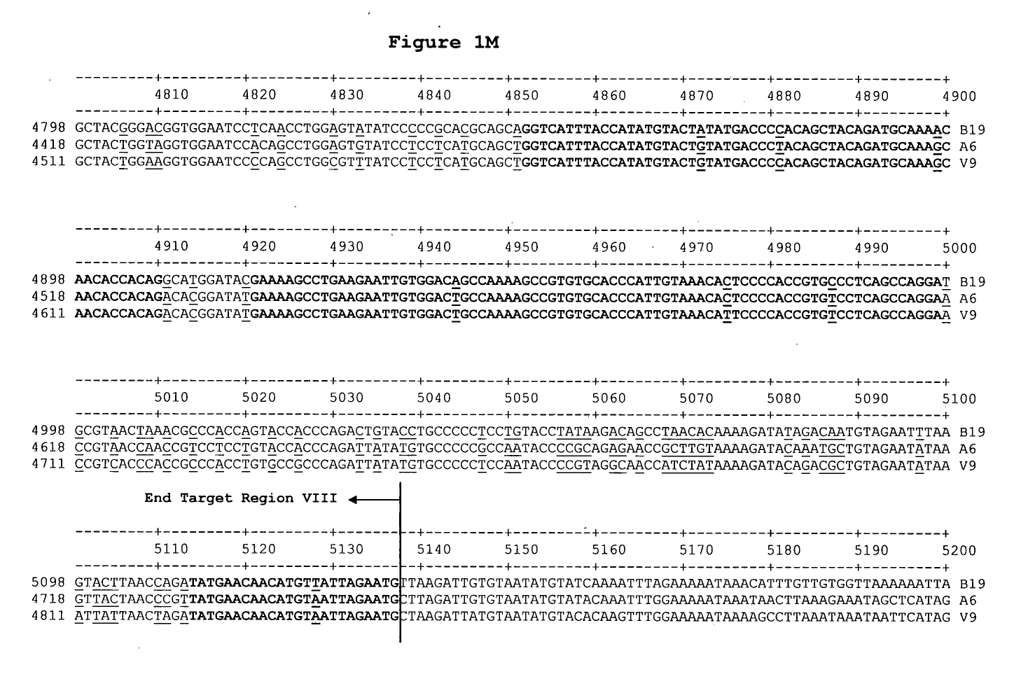

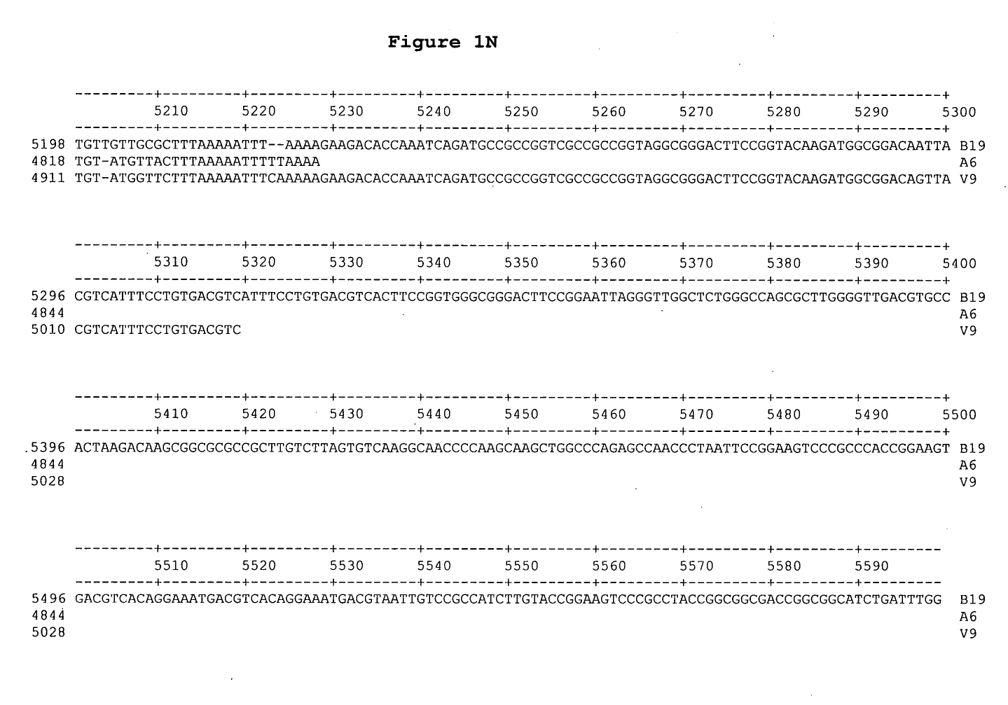

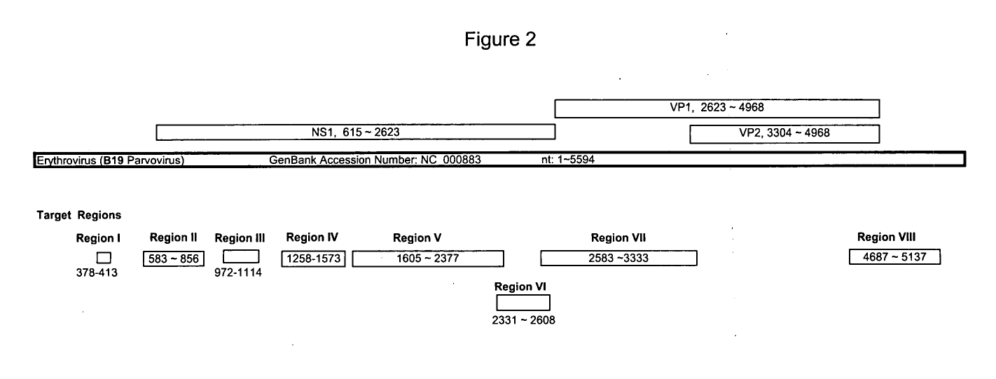

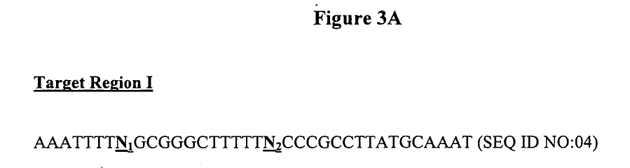

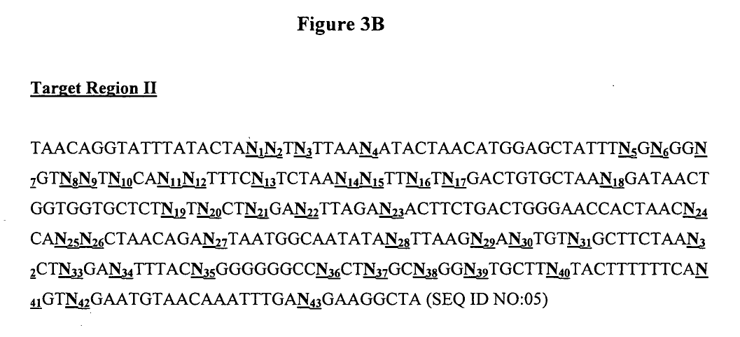

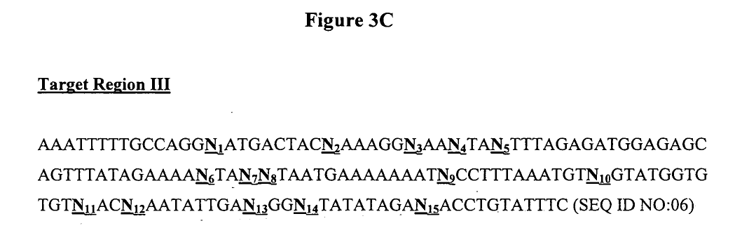

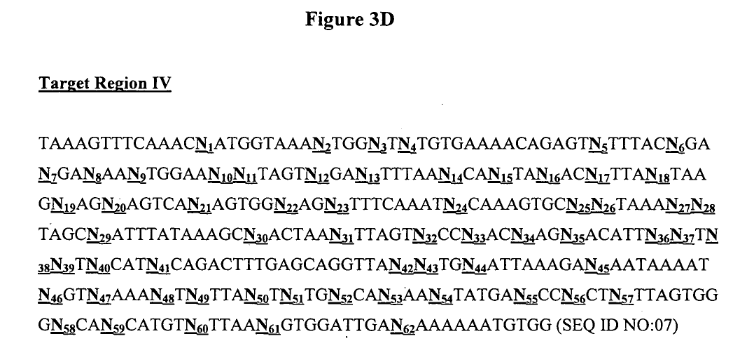

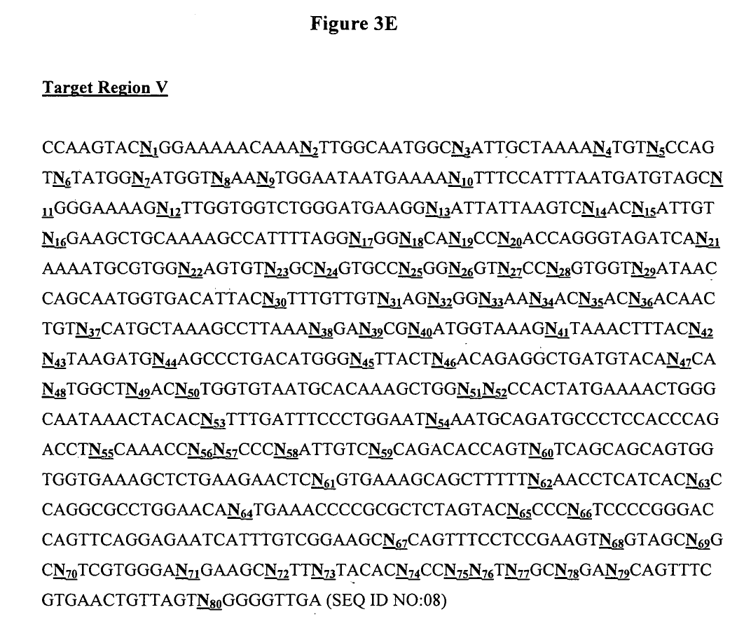

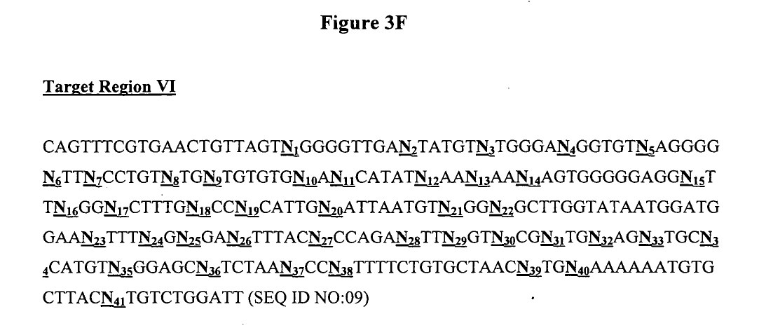

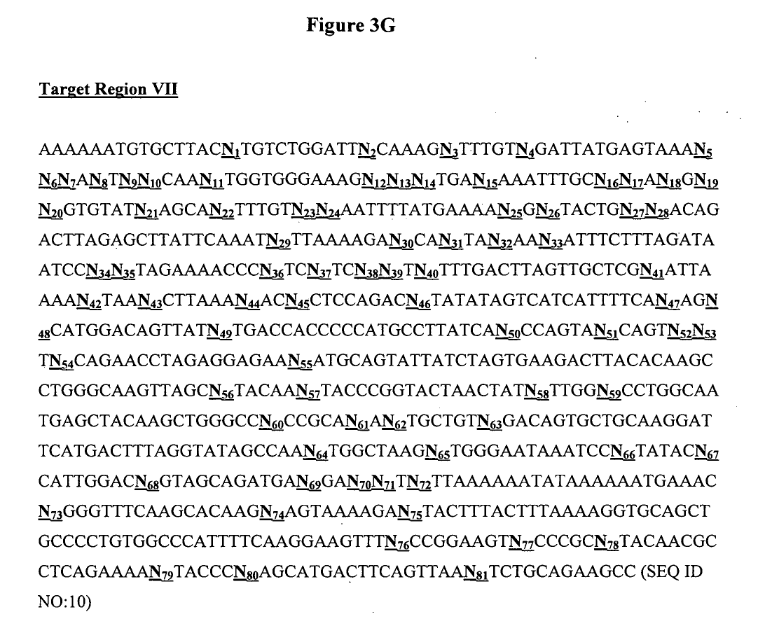

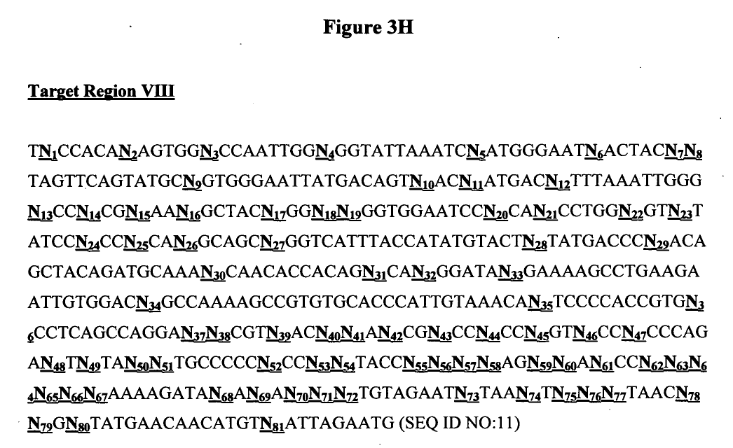

The numbering system on the right side of the figure represents the sequence numbering for each of the three genotypes according to the respective GenBank Accession Numbers for each genotype. The numbering system above the three genotype sequences is the alignment numbering system. The alignment numbering system takes into consideration insertion of spaces (represented as a dash in the nucleotide sequences) in order to optimize alignment of the sequences of the three human EV genotypes. All references to sequences numbering herein are based on the alignment sequence numbering, unless stated otherwise. Variations in the nucleotide sequences between the three genotypes are represented by underlining beneath the variable nucleotides. These variable nucleotides are represented by “N” in the target nucleic acid sequence regions I-VIII, where each N is chosen from a nucleotide at a corresponding position in at least one of a B19 genome, A6 genome, or V9 genome as shown in the alignment of the sequences in FIG. 1. Thus for example, the most 5′ N (i.e., N₁ as shown in FIG. 3A) in Target Region I is generally A (B19), C (A6), or G (V9) as shown in FIG. 1.

Exemplary primers within the Target Regions I-VIII suitable for use in the methods of the invention are indicated by bold typeface. Probes suitable for use in the invention include any sequence positioned within the sequence of an amplification product that would be produced using two selected primers. In some embodiments, the probe is selected to distinguish between the different human EV genotypes. In such embodiments the sequence of the probe is selected such that it corresponds to a region that differs in sequence by one or more nucleotides between the different human EV genotypes to be detected (e.g., the probe region can be selected so as to discriminate between B19, A6, and/or V9 genotypes). In other embodiments, the probe is selected to detect any of the human EV genotypes. In such embodiments, the sequence of the probe is selected such that it corresponds to a region that is the same between the different human EV genotypes to be detected.

FIG. 2 shows the Target Regions I-VIII in relation to the NS1, VP1, and VP2 coding regions of the Human Erythrovirus. Sequence numbering is based on the numbering of the B19 Human Erythrovirus genotype.

FIGS. 3A-3H show the nucleic acid sequences of the Target Regions I-VIII. The Target Regions I-VIII correspond to the target regions denoted in the sequence alignment of FIG. 1. Each variable nucleotide “Nₙ” (where “n” refers to the number of the variant nucleotide within the sequence, e.g,. N₁, N₂, N₃) for the Target Regions I-VIII is chosen from a corresponding position in at least one of the B19 genome, A6 genome, or V9 genome as shown in the alignment of the three genomes in FIG. 1.

### DEFINITIONS

The terms “human erythroviruses,” “human EV,” or “erythrovirus” as used herein refer to a family of viruses formerly known as parvoviruses that have been associated with diseases or conditions in multiple mammals, including dogs and humans. The human erythroviruses are small 22-nm iscoahedral, non-enveloped DNA viruses whose genome includes a linear single-stranded 5.6 kb DNA molecule that encodes two structural proteins, which are designated VP1 and VP2, and a non-structural protein, designated NS-1.

The term “human EV genotypes” as used herein refers to the different human erythrovirus viruses that are sufficiently related in nucleic acid sequence so as to be considered exemplary of a genus. Such human EV genotypes include, but are not limited to, the B19 genotype (GenBank Accession No. NC_{—}000883), the A6/Lali genotype (GenBank Accession No. AY064475), the V9/D91.1 genotype (GenBank Accession No. NC_{—}004295), and the like.

The terms “polynucleotide,” “oligonucleotide,” “nucleic acid” and “nucleic acid molecule” are used interchangeably herein to include a polymeric form of nucleotides, either ribonucleotides or deoxyribonucleotides. This term refers only to the primary structure of the molecule. Thus, the terms include triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA.

Unless specifically indicated otherwise, there is no intended distinction in length between the terms “polynucleotide,” “oligonucleotide,” “nucleic acid” and “nucleic acid molecule” and these terms will be used interchangeably. These terms refer only to the primary structure of the molecule. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. In particular, DNA is deoxyribonucleic acid.

Throughout the specification, abbreviations are used to refer to nucleotides (also referred to as bases), including abbreviations that refer to multiple nucleotides. As used herein, G=guanine, A=adenine, T=thymine, C=cytosine, and U=uracil. In addition, R=a purine nucleotide (A or G); Y=a pyrimidine nucleotide (A or T (U)); S=C or G; W=A or T (U); M=A or C; K=G or T (U); V=A, C or G; and N=any nucleotide (A, T (U), C, or G). Nucleotides can be referred to throughout using lower or upper case letters. It is also understood that nucleotides sequences provided for DNA in the specification also represent nucleotide sequences for RNA, where T is substituted by U.

The terms “deoxyribonucleic acid” and “DNA” as used herein mean a polymer composed of deoxyribonucleotides.

The terms “ribonucleic acid” and “RNA” as used herein refer to a polymer composed of ribonucleotides. Where sequences of a nucleic acid are provided using nucleotides of a DNA sequence, it is understood that such sequences encompass complementary DNA sequences and further also encompass RNA sequences based on the given DNA sequence or its complement, where uracil (U) replaces thymine (T) in the DNA sequence or its complement.

Two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A, T(U), G, and C of one sequence is then aligned with a T(U), A, C, and G, respectively, of the other sequence. RNA sequences can also include complementary G=U or U=G base pairs. Thus, two sequences need not have perfect homology to be “complementary” under the invention. Usually two sequences are sufficiently complementary when at least about 85% (preferably at least about 90%, and most preferably at least about 95%) of the nucleotides share base pair organization over a defined length of the molecule.

As used herein the term “isolated,” when used in the context of an isolated compound, refers to a compound of interest that is in an environment different from that in which the compound naturally occurs. “Isolated” is meant to include compounds that are within samples that are substantially enriched for the compound of interest and/or in which the compound of interest is partially or substantially purified. The term “isolated” encompasses instances in which the recited material is unaccompanied by at least some of the material with which it is normally associated in its natural state, preferably constituting at least about 0.5%, more preferably at least about 5% by weight of the total protein in a given sample. For example, the term “isolated” with respect to a polynucleotide generally refers to a nucleic acid molecule devoid, in whole or part, of sequences normally associated with it in nature; or a sequence, as it exists in nature, but having heterologous sequences in association therewith; or a molecule disassociated from the chromosome.

“Purified” as used herein means that the recited material comprises at least about 75% by weight of the total material, with at least about 80% being preferred, and at least about 90% being particularly preferred. As used herein, the term “substantially pure” refers to a compound that is removed from its natural environment and is at least 60% free, preferably 75% free, and most preferably 90% free from other components with which it is naturally associated.

A polynucleotide “derived from” or “specific for” a designated sequence, such as a target sequence of a target nucleic acid, refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding to, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived or specific for. Polynucleotides that are derived from” or “specific for” a designated sequence include polynucleotides that are in a sense or an antisense orientations relative to the original polynucleotide.

“Homology” refers to the percent similarity between two polynucleotide or two polypeptide moieties. Two DNA, or two polypeptide sequences are “substantially homologous” to each other when the sequences exhibit at least about 50%, preferably at least about 75%, more preferably at least about 80%, at least about 85%, preferably at least about 90%, and most preferably at least about 95% or at least about 98% sequence similarity over a defined length of the molecules. As used herein, substantially homologous also refers to sequences showing complete Identity to the specified DNA or polypeptide sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100.

Readily available computer programs can be used to aid in the analysis of homology and identity, such as Lasergene from DNASTAR, Inc., and ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence homology are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent homology of a particular nucleotide sequence to a reference sequence can be determined using the homology algorithm of Smith and Waterman with a default scoring table and a gap penalty of six nucleotide positions.

Another method of establishing percent homology in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence homology.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the internet on a website sponsored by the National Center for Biotechnology Information (NCBI) and the National Library of Medicine (see the world wide website at ncbi.nlm.gov/cgi-bin/BLAST).

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

“Recombinant” as used herein to describe a nucleic acid molecule refers to a polynucleotide of genomic, cDNA, mammalian, bacterial, viral, semisynthetic, synthetic or other origin which, by virtue of its origin, manipulation, or both is not associated with all or a portion of the polynucleotide with which it is associated in nature. The term “recombinant” as used with respect to a protein or polypeptide means a polypeptide produced by expression of a recombinant polynucleotide.

A “control element” refers to a polynucleotide sequence which aids in the transcription and/or translation of a nucleotide sequence to which it is linked. The term includes promoters, transcription termination sequences, upstream regulatory domains, polyadenylation signals, untranslated regions, including 5′-UTRs and 3′-UTRs and when appropriate, leader sequences and enhancers, which collectively provide for or facilitate the transcription and translation of a coding sequence in a host cell.

A “DNA-dependent DNA polymerase” is an enzyme that synthesizes a complementary DNA copy from a DNA template. Examples include DNA polymerase I from E. coli and bacteriophage T7 DNA polymerase. All known DNA-dependent DNA polymerases require a complementary primer to initiate synthesis. Under suitable conditions, a DNA-dependent DNA polymerase may synthesize a complementary DNA copy from an RNA template.

A “DNA-dependent RNA polymerase” or a “transcriptase” is an enzyme that synthesizes multiple RNA copies from a double-stranded or partially-double stranded DNA molecule having a (usually double-stranded) promoter sequence. The RNA molecules (“transcripts”) are synthesized in the 5′ to 3′ direction beginning at a specific position just downstream of the promoter. Examples of transcriptases are the DNA-dependent RNA polymerase from E. coli and bacteriophages T7, T3, and SP6.

An “RNA-dependent DNA polymerase” or “reverse transcriptase” is an enzyme that synthesizes a complementary DNA copy from an RNA template. All known reverse transcriptases also have the ability to make a complementary DNA copy from a DNA template; thus, they are both RNA- and DNA-dependent DNA polymerases. A primer is required to initiate synthesis with both RNA and DNA templates.

“RNAse H” is an enzyme that degrades the RNA portion of an RNA:DNA duplex. These enzymes may be endonucleases or exonucleases. Most reverse transcriptase enzymes normally contain an RNAse H activity in addition to their polymerase activity. However, other sources of the RNAse H are available without an associated polymerase activity. RNA degradation mediated by an RNAse H may result in separation of RNA from a RNA:DNA complex, or the RNAse H may cut the RNA at various locations such that portions of the RNA melt off or permit enzymes to unwind portions of the RNA.

As used herein, the term “target nucleic acid region” or “target nucleic acid” or “target molecules” refers to a nucleic acid molecule with a “target sequence” to be detected (e.g., by amplification). The target nucleic acid may be either single-stranded or double-stranded and may or may not include other sequences besides the target sequence (e.g., the target nucleic acid may or may not include nucleic acid sequences upstream or 5′ flanking sequence, may or may not include downstream or 3′ flanking sequence, and in some embodiments may not include either upstream (5′) or downstream (3′) nucleic acid sequence relative to the target sequence. Where detection is by amplification, these other sequences in addition to the target sequence may or may not be amplified with the target sequence.

The term “target sequence” or “target nucleic acid sequence” refers to the particular nucleotide sequence of the target nucleic acid to be detected (e.g., through amplification). The target sequence may include a probe-hybridizing region contained within the target molecule with which a probe will form a stable hybrid under desired conditions. The “target sequence” may also include the complexing sequences to which the oligonucleotide primers complex and be extended using the target sequence as a template. Where the target nucleic acid is originally single-stranded, the term “target sequence” also refers to the sequence complementary to the “target sequence” as present in the target nucleic acid. If the “target nucleic acid” is originally double-stranded, the term “target sequence” refers to both the plus (+) and minus (−) strands. Moreover, where sequences of a “target sequence” are provided herein, it is understood that the sequence may be either DNA or RNA. Thus where a DNA sequence is provided, the RNA sequence is also contemplated and is readily provided by substituting “T” of the DNA sequence with “U” to provide the RNA sequence.

The term “primer” or “oligonucleotide primer” as used herein, refers to an oligonucleotide which acts to initiate synthesis of a complementary nucleic acid strand when placed under conditions in which synthesis of a primer extension product is induced, e.g., in the presence of nucleotides and a polymerization-inducing agent such as a DNA or RNA polymerase and at suitable temperature, pH, metal concentration, and salt concentration. Primers are generally of a length compatible with its use in synthesis of primer extension products, and are usually are in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length.

Primers are usually single-stranded for maximum efficiency in amplification, but may alternatively be double-stranded. If double-stranded, the primer is usually first treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

A “primer pair” as used herein refers to first and second primers having nucleic acid sequence suitable for nucleic acid-based amplification of a target nucleic acid. Such primer pairs generally include a first primer having a sequence that is the same or similar to that of a first portion of a target nucleic acid, and a second primer having a sequence that is complementary to a second portion of a target nucleic acid to provide for amplification of the target nucleic acid or a fragment thereof. Reference to “first” and “second” primers herein is arbitrary, unless specifically indicated otherwise. For example, the first primer can be designed as a “forward primer” (which initiates nucleic acid synthesis from a 5′ end of the target nucleic acid) or as a “reverse primer” (which initiates nucleic acid synthesis from a 5′ end of the extension product produced from synthesis initiated from the forward primer). Likewise, the second primer can be designed as a forward primer or a reverse primer.

As used herein, the term “probe” or “oligonucleotide probe”, used interchangeable herein, refers to a structure comprised of a polynucleotide, as defined above, that contains a nucleic acid sequence complementary to a nucleic acid sequence present in the target nucleic acid analyte (e.g., a nucleic acid amplification product). The polynucleotide regions of probes may be composed of DNA, and/or RNA, and/or synthetic nucleotide analogs. Probes are generally of a length compatible with its use in specific detection of all or a portion of a target sequence of a target nucleic acid, and are usually are in the range of between 8 to 100 nucleotides in length, such as 8 to 75, 10 to 74, 12 to 72, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. The typical probe is in the range of between 10-50 nucleotides long, such as 15-45, 18-40, 20-30, 21-28, 22-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length.

Probes contemplated herein include probes that include a detectable label. For example, when an “oligonucleotide probe” is to be used in a 5′ nuclease assay, such as the TaqMan™ assay, the probe includes at least one fluorescer and at least one quencher which is digested by the 5′ endonuclease activity of a polymerase used in the reaction in order to detect any amplified target oligonucleotide sequences. In this context, the oligonucleotide probe will have a sufficient number of phosphodiester linkages adjacent to its 5′ end so that the 5′ to 3′ nuclease activity employed can efficiently degrade the bound probe to separate the fluorescers and quenchers. When an oligonucleotide probe is used in the TMA technique, it will be suitably labeled, as described below.

As used herein, the terms “label” and “detectable label” refer to a molecule capable of detection, including, but not limited to, radioactive isotopes, fluorescers, chemiluminescers, chromophores, enzymes, enzyme substrates, enzyme cofactors, enzyme inhibitors, chromophores, dyes, metal ions, metal sols, ligands (e.g., biotin, avidin, strepavidin or haptens) and the like. The term “fluorescer” refers to a substance or a portion thereof which is capable of exhibiting fluorescence in the detectable range.

The terms “hybridize” and “hybridization” refer to the formation of complexes between nucleotide sequences which are sufficiently complementary to form complexes via Watson-Crick base pairing. Where a primer “hybridizes” with target (template), such complexes (or hybrids) are sufficiently stable to serve the priming function required by, e.g., the DNA polymerase to initiate DNA synthesis.

The term “stringent conditions” refers to conditions under which a primer will hybridize preferentially to, or specifically bind to, its complementary binding partner, and to a lesser extent to, or not at all to, other sequences. Put another way, the term “stringent hybridization conditions” as used herein refers to conditions that are compatible to produce duplexes on an array surface between complementary binding members, e.g., between probes and complementary targets in a sample, e.g., duplexes of nucleic acid probes, such as DNA probes, and their corresponding nucleic acid targets that are present in the sample, e.g., their corresponding mRNA analytes present in the sample.

As used herein, the term “binding pair” refers to first and second molecules that specifically bind to each other, such as complementary polynucleotide pairs capable of forming nucleic acid duplexes. “Specific binding” of the first member of the binding pair to the second member of the binding pair in a sample is evidenced by the binding of the first member to the second member, or vice versa, with greater affinity and specificity than to other components in the sample. The binding between the members of the binding pair is typically noncovalent.

By “selectively bind” is meant that the molecule binds preferentially to the target of interest or binds with greater affinity to the target than to other molecules. For example, a DNA molecule will bind to a substantially complementary sequence and not to unrelated sequences.

A “stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mnM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a probe. Wash conditions used to identify nucleic acids may include, e.g.: a salt concentration of about 0.02 molar at pH 7 and a temperature of at least about 50. ° C. or about 55° C. to about 60° C.; or, a salt concentration of about 0.15 M NaCl at 72° C. for about 15 minutes; or, a salt concentration of about 0.2×SSC at a temperature of at least about 50° C. or about 55. ° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37. ° C. (for 14-base oligos), 48. ° C. (for 17-base oligos), 55° C. (for 20-base oligos), and 60° C. (for 23-base oligos). See Sambrook, Ausubel, or Tijssen (cited below) for detailed descriptions of equivalent hybridization and wash conditions and for reagents and buffers, e.g., SSC buffers and equivalent reagents and conditions.

Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

The “melting temperature” or “Tm” of double-stranded DNA is defined as the temperature at which half of the helical structure of DNA is lost due to heating or other dissociation of the hydrogen bonding between base pairs, for example, by acid or alkali treatment, or the like. The Tₘ of a DNA molecule depends on its length and on its base composition. DNA molecules rich in GC base pairs have a higher Tₘ than those having an abundance of AT base pairs. Separated complementary strands of DNA spontaneously reassociate or anneal to form duplex DNA when the temperature is lowered below the Tₘ. The highest rate of nucleic acid hybridization occurs approximately 25.degree. C. below the Tₘ. The Tₘ may be estimated using the following relationship: Tₘ=69.3+0.41(GC) % (Marmur et al. (1962) J. Mol. Biol. 5:109-118).

As used herein, a “biological sample” refers to a sample of tissue or fluid isolated from a subject, which in the context of the invention generally refers to samples suspected of containing nucleic acid and/or viral particles of human erythrovirus, which samples, after optional processing, can be analyzed in an in vitro assay. Typical samples of interest include, but are not necessarily limited to, respiratory secretions (e.g., samples obtained from fluids or tissue of nasal passages, lung, and the like), blood, plasma, serum, blood cells, fecal matter, urine, tears, saliva, milk, organs, biopsies, and secretions of the intestinal and respiratory tracts. Samples also include samples of in vitro cell culture constituents including but not limited to conditioned media resulting from the growth of cells and tissues in culture medium, e.g., recombinant cells, and cell components.

The term “assessing” includes any form of measurement, and includes determining if an element is present or not. The terms “determining”, “measuring”, “evaluating”, “assessing” and “assaying” are used interchangeably and includes quantitative and qualitative determinations. Assessing may be relative or absolute. “Assessing the presence of” includes determining the amount of something present, and/or determining whether it is present or absent. As used herein, the terms “determining,” “measuring,” and “assessing,” and “assaying” are used interchangeably and include both quantitative and qualitative determinations.

In the context of the methods involving nucleic acid-based amplification of a target sequence, the term “reference range” refers to a range of C_{T} (threshold cycle) values from human EV-negative specimens representative of results that are deemed to indicate that the sample (e.g., a patient specimen) is human EV virus-negative.

In the context of the methods involving nucleic acid-based amplification of a target sequence, the term “reportable range” refers to a range of C_{T} values generated by human EV-positive specimens that are representative of results to be reported as human EV-positive patient specimens.

“Analytical specificity” as used herein refers to the ability of a detection system to specifically detect the target virus and not detect other related viruses, or pathogenic or commensal flora found in the specimen types being validated. For example, “analytical specificity” in reference to assays using human EV primers and a probe refers to the ability of this detection system to specifically amplify and detect the target virus and not detect other related viruses, or pathogenic or commensal flora found in the specimen types being validated.

“Analytical sensitivity” in the context of the methods involving nucleic acid-based amplification of a target sequence refers to the lowest measurable amount of human EV virus target DNA that can be detected for each specimen type validated.

“Precision” refers to the ability of an assay to reproducibly generate the same or comparable result for a given sample.

“Accuracy” refers to the ability of an assay to correctly detect a target molecule in a blinded panel containing both positive and negative specimens.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to a “oligonucleotide primer” includes a plurality of such primers and reference to “primer” includes reference to one or more the primers and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The practice of the present invention will employ, unless otherwise indicated, conventional methods of chemistry, biochemistry, recombinant DNA techniques and virology, within the skill of the art. Such techniques are explained fully in the literature. See, e.g., Fundamental Virology, 2nd Edition, vol. I & II (B. N. Fields and D. M. Knipe, eds.); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Oligonucleotide Synthesis (N. Gait, ed., 1984); A Practical Guide to Molecular Cloning (1984).

The invention will now be described in more detail.

### DETAILED DESCRIPTION OF TH INVENTION

The invention is based on the discovery of consensus target nucleic acid regions within the human erythrovirus (human EV) genome that include target nucleic acid sequences (also referred to herein as target sequences) for detection of the human EV in a sample, particularly a biological sample, with specificity and sensitivity. In particular the detection of one or more target nucleic acid sequence regions allows for detection of human EV in a sample, in general, while also being able to discriminate between different genotypes of human EV, such as the human EV genotypes B19, A6, and V9. The specificity and simplicity of these assays facilitate rapid, reliable and inexpensive assays for detection of human EV in general and discrimination between different human EV genotypes, such as B19, A6, and V9. The subject invention finds use in a variety of different applications, including research, medical, and diagnostic applications.

In general, the subject methods provide for detection of human EV in a sample, such as a biological sample, by detection of a target nucleic acid region of the human EV genome. Eight such target nucleic acid regions are described herein, termed as Target Regions I-VIII as designated in FIG. 1. FIG. 2 provides a schematic showing the relative position of the Target Regions within the B19 genome.

In some embodiments, the subject methods provide for detection of any human EV genotype, e.g., B19, A6, or V9, in a sample, such a biological sample. In such embodiments, the subject methods detect a target nucleic acid region, or fragment thereof, by using primers and probe that correspond to sequences within the target region. Exemplary primers within the Target Regions I-VIII suitable for use in the methods of the invention are indicated by bold typeface in FIG. 1. Probes suitable for use in the invention include any sequence positioned within the sequence of an amplification product that would be produced using selected primers. A probe suitable for use with such an embodiment is selected such that it corresponds to a region that shares a nucleotide sequence between the different human EV genotypes to be detected.

In other embodiments, the subject methods provide for detection and discrimination between different human EV genotypes, e.g., B19, A6, or V9, in a sample, such a biological sample, i.e., to provide for genotype-specific detection. In such embodiments, the subject methods detect a target nucleic acid region, or fragment thereof, by using primers and probe that correspond to sequences within the target region. Exemplary primers within the Target Regions I-VIII suitable for use in the methods of the invention are indicated by bold typeface in FIG. 1. Probes suitable for use in the invention include any sequence positioned within the sequence of an amplification product that would be produced using selected primers. In such embodiments the sequence of the probe is selected such that it corresponds to a region that differs in sequence by one or more nucleotides between the different human EV genotypes to be detected (e.g., the probe region can be selected so as to discriminate between B19, A6, and/or V9 genotypes). Variations in the nucleotide sequences between the three genotypes are represented in FIG. 1 by circles surrounding the variable nucleotides.

We note that the sequences provided herein, and particularly the consensus sequences are provided as DNA sequences. It is understood that the DNA sequences provided may be single stranded or double stranded, and as such the description of the DNA sequences below is intended to also provide the complementary sequence as well.

The compositions and methods of the invention will now be described in more detail.

### Target Nucleic Acid Regions

Target nucleic acid sequence regions were identified by alignment of the B19, A6, and V9 genomes. The present invention provides for identification of human EV in a sample, such as a biological sample, by detecting one or more target nucleic acid region or a portion thereof. In general, detection is by nucleic acid amplification, which in some embodiments is followed by detection of the amplification product using a hybridization probe. The target nucleic acid regions are provided in FIGS. 3A to 3H and are described in further detail below.

It will be appreciated that since human EVs contain a single-stranded DNA genome from which double-stranded DNA is generated during replication, and further since RNA is produced from such human EV DNA during viral replication, the primers and probes described herein encompass those having the nucleic acid sequence described herein, as well as primers and probes having the complement of such nucleic acid sequences.

Furthermore, it will be understood that primer pairs useful in the invention include a first primer having a sequence that is the same or similar to that of the human EV sequence provided herein, and a second primer having a sequence that is complementary to the human EV sequence provided herein to provide for amplification of an human EV target nucleic acid region described herein or a fragment thereof (e.g., the first primer is a “forward” primer and the second primer is a “reverse” primer). It will be further understood that primer pairs useful in the invention also include a first primer having a sequence that is complementary to that of the human EV sequence provided herein, and a second primer having a sequence that is the same or similar to the human EV sequence provided herein to provide for amplification of an human EV target nucleic acid region described herein or a fragment thereof (e.g., the first primer is a “reverse” primer and the second primer is a “forward” primer).

It also will be understood that the nucleic acid sequence of probes described herein can be the same or similar to that of the human EV sequence provided or a complement thereof. In addition, primers described herein can also be used as probes, e.g., to detect an amplification product.

Target Region I

In one embodiment, the invention provides for detection of different human EV genotypes, e.g. B19, A6, and V9, in a sample, such as a biological sample, by detection of target nucleic acid sequence region I (FIG. 1, Target Region I, alignment position 378-413) as follows:

(SEQ ID NO: 04)

AAATTTTNGCGGGCTTTTTNCCCGCCTTATGCAAAT

or a complement thereof, or a fragment thereof, wherein the variable nucleotide N is a nucleotide at the corresponding position in at least one of the B19 genome, A6 genome, or V9 genome as shown in the alignment of the three genomes in FIG. 1.

Exemplary nucleic acid sequences suitable for design of primers for amplification of a Target Region I nucleic acid, and suitable for use in the methods of the invention, are indicated by bold typeface in FIG. 1. Suitable sequences for primers for amplification of Target Region I nucleic acid correspond to nucleotides 1-19 and 21-36 of the 36 nucleotide sequence of SEQ ID NO:04 (FIG. 1), or a complement thereof.

Probes suitable for use in the invention can be designed from any sequence positioned within the sequence of an amplification product that would be produced using two selected primers. In some embodiments, the probe is selected to distinguish between (i.e., discriminate between) the different human EV genotypes. In such embodiments the sequence of the probe is selected so that it corresponds to a region that differs in sequence by one or more nucleotides between the different human EV genotypes to be detected (e.g., the probe region can be selected so as to discriminate between B19, A6, and/or V9 genomes). Variations in the nucleotide sequences between the three genotypes are represented by circles surrounding the variable nucleotides in FIG. 1 and denoted by the variable N in SEQ ID NO:04.

In other embodiments, the probe is selected to detect any of the human EV genotypes which may or may not include discrimination between the human EV genotypes. Such probes can provide for detection of, for example, all three of B19 nucleic acid, V9 nucleic acid, and A6 nucleic acid; B19 nucleic acid and V9 nucleic acid; V9 nucleic acid and A6 nucleic acid; B19 nucleic acid, and A6 nucleic acid; or any one of B19 nucleic acid, V9 nucleic acid, and A6 nucleic acid. In such embodiments, the sequence of the probe is selected such that it corresponds to a region that is the same between the different human EV genotypes to be detected.

In one embodiment, detection of target region I nucleic acid involves production of an amplification product of at least 30, at least 28, at least 26, at least 24, at least 22, at least 20 consecutive nucleotides of SEQ ID NO:04.

The methods of the invention can involve detection of target region I nucleic acid either alone or in combination with detection of one or more of target regions II-VIII as described herein. For example, detection of target region I and detection of target region II; or detection of target region I and detection of target region III; or detection of target region I and detection of target region IV; or detection of target region I and detection of target region V; and the like. In addition, the methods of the invention can involve detection of 3 or more, or 4 or more, or 5 or more target regions, and the like. For example, the methods of the invention can involve detection of target region I, target region II, target region III; or detection of target region I, target region IV, target region VIII; or detection of target region I, target region II, target region VII; and the like. It will be understood that detection of all combination of target regions I-VIII are contemplates by the present methods.

Exemplary primers and probes are discussed in greater detail below.

Target Region II

In another embodiment, the invention provides for detection of different human EV genotypes, e.g., B19, A6, and V9, in a sample, such as a biological sample, by detection of target nucleic acid sequence region II (FIG. 1, Target Region II, alignment position 583-856) as follows:

| | |
|---|---|
| TAACAGGTATTTATACTANNTNTTAANATACTAACATGGAGCTATTTNGNGGN | (SEQ ID NO: 05) |
| GTNNTNCANNTTTCNTCTAANNTTNTNGACTGTGCTAANGATAACTGGTGGTG | |
| CTCTNTNCTNGANTTAGANACTTCTGACTGGGAACCACTAACNCANNCTAACA | |
| GANTAATGGCAATATANTTAAGNANTGTNGCTTCTAANCTNGANTTTACNGGG | |
| GGGCCNCTNGCN+E GGNTGCTTNTACTTTTTTCANGTNGAATGTAACAAATTTGA | |
| NGAAGGCTA | |

or a complement thereof, or a fragment thereof, wherein the variable nucleotide N is selected from a nucleotide at the corresponding position in either the B19 genome, A6 genome, or V9 genome as shown in the alignment of the three genomes in FIG. 1.

Exemplary nucleic acid sequences suitable for design of primers for amplification of a Target Region II nucleic acid, and suitable for use in the methods of the invention, are indicated by bold typeface in FIG. 1. Suitable sequences for primers for amplification of Target Region II nucleic acid correspond to nucleotides 1-18, 29-47, 81-110, 126-148, 154-175, and 249-274 of the 274 nucleotide sequence of SEQ ID NO:05 (FIG. 1), or a complement thereof.

Probes suitable for use in the invention can be designed from any sequence positioned within the sequence of an amplification product that would be produced using two selected primers. In some embodiments, the probe is selected to distinguish between the different human EV genotypes. In such embodiments the sequence of the probe is selected so that it corresponds to a region that differs in sequence by one or more nucleotides between the different human EV genotypes to be detected (e.g., the probe region can be selected so as to discriminate between B19, A6, and/or V9 genotypes). Variations in the nucleotide sequences between the three genotypes are represented by circles surrounding the variable nucleotides in FIG. 1 and denoted by the variable N in SEQ ID NO:05.

In other embodiments, the probe is selected to detect any of the human EV genotypes which may or may not include discrimination between the human EV genotypes. Such probes can provide for detection of, for example, all three of B19 nucleic acid, V9 nucleic acid, and A6 nucleic acid; B19 nucleic acid and V9 nucleic acid; V9 nucleic acid and A6 nucleic acid; B19 nucleic acid, and A6 nucleic acid; or any one of B19 nucleic acid, V9 nucleic acid, and A6 nucleic acid. In such embodiments, the sequence of the probe is selected such that it corresponds to a region that is the same between the different human EV genotypes to be detected.

In one embodiment, detection of target region II nucleic acid involves production of an amplification product of at least 274, at least 250, at least 225, at least 200, at least 175, at least 150, at least 125, at least 100, at least 90, at least 80, at least 70, at least 60, at least 50, at least 40, at least 30, at least 26, at least 24, at least 22, at least 20 consecutive nucleotides of SEQ ID NO:05.

The methods of the invention can involve detection of target region I nucleic acid either alone or in combination with detection of one or more of target regions I and III-VIII as described herein. For example, detection of target region I and detection of target region II; or detection of target region II and detection of target region III; or detection of target region II and detection of target region IV; or detection of target region II and detection of target region V; and the like. In addition, the methods of the invention can involve detection of 3 or more, or 4 or more, or 5 or more target regions, and the like. For example, the methods of the invention can involve detection of target region I, target region II, target region III; or detection of target region II, target region IV, target region VII; or detection of target region I, target region II, target region VII; and the like. It will be understood that detection of all combination of target regions I-VIII are contemplates by the present methods.

Exemplary primers and probes are discussed in greater detail below.

Target Region III

In another embodiment, the invention provides for detection of different human EV genotypes, e.g., B19, A6, and V9, in a sample, such as a biological sample, by detection of target nucleic acid sequence region III (FIG. 1, Target Region III, alignment position 972-1114) as follows:

| | |
|---|---|
| AAATTTTTGCCAGGNATGACTACNAAAGGNAANTANTTTAGAGATGGAGAGC | (SEQ ID NO: 06) |
| AGTTTATAGAAAANTANNTAATGAAAAAAATNCCTTTAAATGTNGTATGGTGT | |
| GTNACNAATATTGANGGNTATATAGANACCTGTATTTC | |

or a complement thereof, or a fragment thereof, wherein the variable nucleotide N is selected from a nucleotide at the corresponding position in either the B19 genome, A6 genome, or V9 genome as shown in the alignment of the three genomes in FIG. 1.

Exemplary nucleic acid sequences suitable for design of primers for amplification of a Target Region III nucleic acid, and suitable for use in the methods of the invention, are indicated by bold typeface in FIG. 1. Suitable sequences for primers for amplification of Target Region III nucleic acid correspond to nucleotides 1-23, 36-65, 71-106, and 124-143 of the 143 nucleotide sequence of SEQ ID NO:06 (FIG. 1), or a complement thereof.

Probes suitable for use in the invention can be designed from any sequence positioned within the sequence of an amplification product that would be produced using two selected primers. In some embodiments, the probe is selected to distinguish between the different human EV genotypes. In such embodiments the sequence of the probe is selected so that it corresponds to a region that differs in sequence by one or more nucleotides between the different human EV genotypes to be detected (e.g., the probe region can be selected so as to discriminate between B19, A6, and/or V9 genomes). Variations in the nucleotide sequences between the three genotypes are represented by circles surrounding the variable nucleotides in FIG. 1 and denoted by the variable N in SEQ ID NO:06.

In other embodiments, the probe is selected to detect any of the human EV genotypes, which may or may not include discrimination between the human EV genotypes. Such probes can provide for detection of, for example, all three of B19 nucleic acid, V9 nucleic acid, and A6 nucleic acid; B19 nucleic acid and V9 nucleic acid; V9 nucleic acid and A6 nucleic acid; B19 nucleic acid, and A6 nucleic acid; or any one of B19 nucleic acid, V9 nucleic acid, and A6 nucleic acid. In such embodiments, the sequence of the probe is selected such that it corresponds to a region that is the same between the different human EV genotypes to be detected.

In one embodiment, detection of target region III nucleic acid involves production of an amplification product of at least 143, at least 130, at least 120, at least 110, at least 100, at least 90, at least 80, at least 70, at least 60, at least 50, at least 40, at least 30, at least 26, at least 24, at least 22, at least 20 consecutive nucleotides of SEQ ID NO:06.

The methods of the invention can involve detection of target region I nucleic acid either alone or in combination with detection of one or more of target regions I-II and IV-VIII as described herein. For example, detection of target region I and detection of target region III; or detection of target region III and detection of target region VIII; or detection of target region III and detection of target region IV; or detection of target region III and detection of target region V; and the like. In addition, the methods of the invention can involve detection of 3 or more, or 4 or more, or 5 or more target regions, and the like. For example, the methods of the invention can involve detection of target region I, target region II, target region III; or detection of target region III, target region IV, target region VII; or detection of target region II, target region III, target region VII; and the like. It will be understood that detection of all combination of target regions I-VIII are contemplates by the present methods.

Exemplary primers and probes are discussed in greater detail below.

Target Region IV

In another embodiment, the invention provides for detection of different human EV genotypes, e.g., B19, A6, and V9, in a sample, such as a biological sample, by detection of target nucleic acid sequence region IV (FIG. 1, Target Region IV, alignment position 1258-1573) as follows:

| | |
|---|---|
| TAAAGTTTCAAACNATGGTAAANTGGNTNTGTGAAAACAGAGTNTTTACNGAN | (SEQ ID NO: 07) |
| GANAANTGGAANNTAGTNGANTTTAANCANTANACNTTANTAAGNAGNAGTC | |
| ANAGTGGNAGNTTTCAAATNCAAAGTGCNNTAAANNTAGCNATTTATAAAGC | |
| NACTAANTTAGTNCCNACNAGNACATTNNTNNTNCATNCAGACTTTGAGCAGG | |
| TTANNTGNATTAAAGANAATAAAATNGTNAAANTNTTANTNTGNCANAANTAT | |
| GANCCNCTNTTAGTGGGNCANCATGTNTTAANGTGGATTGANAAAAAATGTGG | |

or a complement thereof, or a fragment thereof, wherein the variable nucleotide N is selected from a nucleotide at the corresponding position in either the B19 genome, A6 genome, or V9 genome as shown in the alignment of the three genomes in FIG. 1.

Exemplary nucleic acid sequences suitable for design of primers for amplification of a Target Region IV nucleic acid, and suitable for use in the methods of the invention, are indicated by bold typeface in FIG. 1. Suitable sequences for primers for amplification of Target Region IV nucleic acid correspond to nucleotides 1-22, 30-43, 117-133, 196-213, 219-235, and 296-316 of the 316 nucleotide sequence of SEQ ID NO:07 (FIG. 1), or a complement thereof.

Probes suitable for use in the invention can be designed from any sequence positioned within the sequence of an amplification product that would be produced using two selected primers. In some embodiments, the probe is selected to distinguish between the different human EV genotypes. In such embodiments the sequence of the probe is selected so that it corresponds to a region that differs in sequence by one or more nucleotides between the different human EV genotypes to be detected (e.g., the probe region can be selected so as to discriminate between B19, A6, and/or V9 genomes). Variations in the nucleotide sequences between the three genotypes are represented by circles surrounding the variable nucleotides in FIG. 1 and denoted by the variable N in SEQ ID NO:07.

In other embodiments, the probe is selected to detect any of the human EV genotypes, which may or may not include discrimination between the human EV genotypes. Such probes can provide for detection of, for example, all three of B19 nucleic acid, V9 nucleic acid, and A6 nucleic acid; B19 nucleic acid and V9 nucleic acid; V9 nucleic acid and A6 nucleic acid; B19 nucleic acid, and A6 nucleic acid; or any one of B19 nucleic acid, V9 nucleic acid, and A6 nucleic acid. In such embodiments, the sequence of the probe is selected such that it corresponds to a region that is the same between the different human EV genotypes to be detected.

In one embodiment, detection of target region IV nucleic acid involves production of an amplification product of at least 316, at least 300, at least 275, at least 250, at least 225, at least 200, at least 175, at least 150, at least 125, at least 100, at least 90, at least 80, at least 70, at least 60, at least 50, at least 40, at least 30, at least 26, at least 24, at least 22, at least 20 consecutive nucleotides of SEQ ID NO:07.

The methods of the invention can involve detection of target region I nucleic acid either alone or in combination with detection of one or more of target regions I-III and V-VIII as described herein. For example, detection of target region IV and detection of target region III; or detection of target region IV and detection of target region VIII; or detection of target region III and detection of target region IV; or detection of target region IV and detection of target region V; and the like. In addition, the methods of the invention can involve detection of 3 or more, or 4 or more, or 5 or more target regions, and the like. For example, the methods of the invention can involve detection of target region IV, target region II, target region III; or detection of target region III, target region IV, target region VII; or detection of target region IV, target region III, target region VII; and the like. It will be understood that detection of all combination of target regions 1-VIII are contemplates by the present methods.

Exemplary primers and probes are discussed in greater detail below.

Target Region V

In another embodiment, the invention provides for detection of different human EV genotypes, e.g., B19, A6, and V9, in a sample, such as a biological sample, by detection of target nucleic acid sequence region V (FIG. 1, Target Region V, alignment position 1605-2377) as follows:

| | |
|---|---|
| CCAAGTACNGGAAAAACAAANTTGGCAATGGCNATTGCTAAAANTGTNCCAG | (SEQ ID NO: 08) |
| TNTATGGNATGGTNAANTGGAATAATGAAAANTTTCCATTTAATGATGTAGCN | |
| GGGAAAAGNTTGGTGGTCTGGGATGAAGGNATTATTAAGTCNACNATTGTNGA | |
| AGCTGCAAAAGCCATTTTAGGNGGNCANCCNACCAGGGTAGATCANAAAATG | |
| CGTGGNAGTGTNGCNGTGCCNGGNGTNCCNGTGGTNATAACCAGCAATGGTG | |
| ACATTACNTTTGTTGTNAGNGGNAANACNACNACAACTGTNCATGCTAAAGCC | |
| TTAAANGANCGNATGGTAAAGNTAAACTTTACNNTAAGATGNAGCCCTGACAT | |
| GGGNTTACTNACAGAGGCTGATGTACANCANTGGCTNACNTGGTGTAATGCAC | |
| AAAGCTGGNNCCACTATGAAAACTGGGCAATAAACTACACNTTTGATTTCCCT | |
| GGAATNAATGCAGATGCCCTCCACCCAGACCTNCAAACCNNCCCNATTGTCNC | |
| AGACACCAGTNTCAGCAGCAGTGGTGGTGAAAGCTCTGAAGAACTCNGTGAA | |
| AGCAGCTTTTTNAACCTCATCACNCCAGGCGCCTGGAACANTGAAACCCCGCG | |
| CTCTAGTACNCCCNTCCCCGGGACCAGTTCAGGAGAATCATTTGTCGGAAGCN | |
| CAGTTTCCTCCGAAGTNGTAGCNGCNTCGTGGGANGAAGCNTTNTACACNCCN | |
| NTNGCNGANCAGTTTCGTGAACTGTTAGTNGGGGTTGA | |

or complement thereof, or a fragment thereof, wherein the variable nucleotide N is selected from a nucleotide at the corresponding position in either the B19 genome, A6 genome, or V9 genome as shown in the alignment of the three genomes in FIG. 1.

Exemplary nucleic acid sequences suitable for design of primers for amplification of a Target Region V nucleic acid, and suitable for use in the methods of the invention, are indicated by bold typeface in FIG. 1. Suitable sequences for primers for amplification of Target Region V nucleic acid correspond to nucleotides 1-43,70-104, 116-131, 158-179, 190-215, 247-278, 295-320, 328-347, 358-371, 379-395, 411-429, 432-513, 520-641, 647-701, and 748-776 of the 776 nucleotide sequence of SEQ ID NO:08 (FIG. 1), or a complement thereof.

Probes suitable for use in the invention can be designed from any sequence positioned within the sequence of an amplification product that would be produced using two selected primers. In some embodiments, the probe is selected to distinguish between the different human EV genotypes. In such embodiments the sequence of the probe is selected so that it corresponds to a region that differs in sequence by one or more nucleotides between the different human EV genotypes to be detected (e.g., the probe region can be selected so as to discriminate between B19, A6, and/or V9 genomes). Variations in the nucleotide sequences between the three genotypes are represented by circles surrounding the variable nucleotides in FIG. 1 and denoted by the variable N in SEQ ID NO:08.

In other embodiments, the probe is selected to detect any of the human EV genotypes which may or may not include discrimination between the human EV genotypes. Such probes can provide for detection of, for example, all three of B19 nucleic acid, V9 nucleic acid, and A6 nucleic acid; B19 nucleic acid and V9 nucleic acid; V9 nucleic acid and A6 nucleic acid; B19 nucleic acid, and A6 nucleic acid; or any one of B19 nucleic acid, V9 nucleic acid, and A6 nucleic acid. In such embodiments, the sequence of the probe is selected such that it corresponds to a region that is the same between the different human EV genotypes to be detected.

In one embodiment, detection of target region V nucleic acid involves production of an amplification product of at least 776, at least 750, at least 725, at least 700, at least 675, at least 650, at least 625, at least 600, at least 575, at least 550, at least 525, at least 500, at least 475, at least 450, at least 425, at least 400, at least 375, at least 350, at least 325, at least 300, at least 275, at least 250, at least 225, at least 200, at least 175, at least 150, at least 125, at least 100, at least 90, at least 80, at least 70, at least 60, at least 50, at least 40, at least 30, at least 26, at least 24, at least 22, at least 20 consecutive nucleotides of SEQ ID NO:08.

The methods of the invention can involve detection of target region I nucleic acid either alone or in combination with detection of one or more of target regions I-IV and VI-VIII as described herein. For example, detection of target region V and detection of target region III; or detection of target region V and detection of target region VIII; or detection of target region III and detection of target region V; or detection of target region IV and detection of target region V; and the like. In addition, the methods of the invention can involve detection of 3 or more, or 4 or more, or 5 or more target regions, and the like. For example, the methods of the invention can involve detection of target region V, target region II, target region III; or detection of target region III, target region V, target region VII; or detection of target region V, target region III, target region VII; and the like. It will be understood that detection of all combination of target regions I-VIII are contemplates by the present methods.

Exemplary primers and probes are discussed in greater detail below.

Target Region VI

In another embodiment, the invention provides for detection of different human EV genotypes, e.g., B19, A6, and V9, in a sample, such as a biological sample, by detection of target nucleic acid sequence region VI (FIG. 1, Target Region VI, alignment position 2531-2608) as follows:

| | |
|---|---|
| CAGTTTCGTGAACTGTTAGTNGGGGTTGANTATGTNTGGGANGGTGTNAGGGG | (SEQ ID NO: 09) |
| NTTNCCTGTNTGNTGTGTGNANCATATNAANAANAGTGGGGGAGGNTTNGGN | |
| CTTTGNCCNCATTGNATTAATGTNGGNGCTTGGTATAATGGATGGAANTTTNG | |
| NGANTTTACNCCAGANTTNGTNCGNTGNAGNTGNCATGTNGGAGCNTCTAANC | |
| CNTTTTCTGTGCTAACNTGNAAAAAATGTGCTTACNTGTCTGGATT | |

or a complement thereof, or a fragment thereof, wherein the variable nucleotide N is selected from a nucleotide at the corresponding position in either the B19 genome, A6 genome, or V9 genome as shown in the alignment of the three genomes in FIG. 1. As shown FIGS. 1 and 2, a portion of the 5′ end of Target Region VI overlaps with a portion of the 3′ end of Target Region V, and a portion of the 3′ end of Target Region VI overlaps with a portion of the 5′ end of Target Region VII.

Exemplary nucleic acid sequences suitable for design of primers for amplification of a Target Region VI nucleic acid, and suitable for use in the methods of the invention, are indicated by bold typeface in FIG. 1. Suitable sequences for primers for amplification of Target Region VI nucleic acid correspond to nucleotides 1-29, 126-168, and 232-257 of the 257 nucleotide sequence of SEQ ID NO:09 (FIG. 1), or a complement thereof.

Probes suitable for use in the invention can be designed from any sequence positioned within the sequence of an amplification product that would be produced using two selected primers. In some embodiments, the probe is selected to distinguish between the different human EV genotypes. In such embodiments the sequence of the probe is selected so that it corresponds to a region that differs in sequence by one or more nucleotides between the different human EV genotypes to be detected (e.g., the probe region can be selected so as to discriminate between B19, A6, and/or V9 genomes). Variations in the nucleotide sequences between the three genotypes are represented by circles surrounding the variable nucleotides in FIG. 1 and denoted by the variable N in SEQ ID NO:09.

In other embodiments, the probe is selected to detect any of the human EV genotypes which may or may not include discrimination between the human EV genotypes. Such probes can provide for detection of, for example, all three of B19 nucleic acid, V9 nucleic acid, and A6 nucleic acid; B19 nucleic acid and V9 nucleic acid; V9 nucleic acid and A6 nucleic acid; B19 nucleic acid, and A6 nucleic acid; or any one of B19 nucleic acid, V9 nucleic acid, and A6 nucleic acid. In such embodiments, the sequence of the probe is selected such that it corresponds to a region that is the same between the different human EV genotypes to be detected.

In one embodiment, detection of target region VI nucleic acid involves production of an amplification product of at least 257, at least 225, at least 200, at least 175, at least 150, at least 125, at least 100, at least 90, at least 80, at least 70, at least 60, at least 50, at least 40, at least 30, at least 26, at least 24, at least 22, at least 20 consecutive nucleotides of SEQ ID NO:09.

The methods of the invention can involve detection of target region I nucleic acid either alone or in combination with detection of one or more of target regions I-V and VII-VIII as described herein. For example, detection of target region VI and detection of target region III; or detection of target region VI and detection of target region VIII; or detection of target region III and detection of target region VI; or detection of target region VI and detection of target region V; and the like. In addition, the methods of the invention can involve detection of 3 or more, or 4 or more, or 5 or more target regions, and the like. For example, the methods of the invention can involve detection of target region VI, target region II, target region III; or detection of target region III, target region VI, target region VII; or detection of target region VI, target region III, target region VII; and the like. It will be understood that detection of all combination of target regions I-VIII are contemplates by the present methods.

Exemplary primers and probes are discussed in greater detail below.

Target Region VII

In another embodiment, the invention provides for detection of different human EV genotypes, e.g., B19, A6, and V9, in a sample, such as a biological sample, by detection of target nucleic acid sequence region VII (FIG. 1, Target Region VII, alignment position 2583-3333) as follows:

| | |
|---|---|
| AAAAAATGTGCTTACNTGTCTGGATTNCAAAGNTTTGTNGATTATGAGTAAAN | (SEQ ID NO: 10) |
| NNANTNNCAANTGGTGGGAAAGNNNTGANAAATTTGCNNANGNNGTGTATNA | |
| GCANTTTGTNNAATTTTATGAAAANGNTACTGNNACAGACTTAGAGCTTATTC | |
| AAATNTTAAAAGANCANTANAANATTTCTTTAGATAATCCNNTAGAAAACCCN | |
| TCNTCNNTNTTTGACTTAGTTGCTCGNATTAAAANTAANCTTAAANACNCTCCA | |
| GACNTATATAGTCATCATTTTCANAGNCATGGACAGTTATNTGACCACCCCCA | |
| TGCCTTATCANCCAGTANCAGTNNTNCAGAACCTAGAGGAGAANATGCAGTAT | |
| TATCTAGTGAAGACTTACACAAGCCTGGGCAAGTTAGCNTACAANTACCCGGT | |
| ACTAACTATNTTGGNCCTGGCAATGAGCTACAAGCTGGGCCNCCGCANANTGC | |
| TGTNGACAGTGCTGCAAGGATTCATGACTTTAGGTATAGCCAANTGGCTAAGN | |
| TGGGAATAAATCCNTATACNCATTGGACNGTAGCAGATGANGANNTNTTAAA | |
| AAATATAAAAAATGAAACNGGGTTTCAAGCACAAGNAGTAAAAGANTACTTT | |
| ACTTTAAAAGGTGCAGCTGCCCCTGTGGCCCATTTTCAAGGAAGTTTNCCGGA | |
| AGTNCCCGCNTACAACGCCTCAGAAAANTACCCNAGCATGACTTCAGTTAANT | |
| CTGCAGAAGCC | |

or complement thereof, or a fragment thereof, wherein the variable nucleotide N is selected from a nucleotide at the corresponding position in either the B19 genome, A6 genome, or V9 genome as shown in the alignment of the three genomes in FIG. 1.

Exemplary nucleic acid sequences suitable for design of primers for amplification of a Target Region VII nucleic acid, and suitable for use in the methods of the invention, are indicated by bold typeface in FIG. 1. Suitable sequences for primers for amplification of Target Region VII nucleic acid correspond to nucleotides 1-52, 117-129, 133-171, 181-213, 221-245, 251-288, 293-335, 345-409, 417-465, 475-543, 551-570, 578-690, 698-714, and 722-751 of the 751 nucleotide sequence of SEQ ID NO:10 (FIG. 1), or a complement thereof.

Probes suitable for use in the invention can be designed from any sequence positioned within the sequence of an amplification product that would be produced using two selected primers. In some embodiments, the probe is selected to distinguish between the different human EV genotypes. In such embodiments the sequence of the probe is selected so that it corresponds to a region that differs in sequence by one or more nucleotides between the different human EV genotypes to be detected (e.g., the probe region can be selected so as to discriminate between B19, A6, and/or V9 genomes). Variations in the nucleotide sequences between the three genotypes are represented by circles surrounding the variable nucleotides in FIG. 1 and denoted by the variable N in SEQ ID NO:10.

In other embodiments, the probe is selected to detect any of the human EV genotypes which may or may not include discrimination between the human EV genotypes. Such probes can provide for detection of, for example, all three of B19 nucleic acid, V9 nucleic acid, and A6 nucleic acid; B19 nucleic acid and V9 nucleic acid; V9 nucleic acid and A6 nucleic acid; B19 nucleic acid, and A6 nucleic acid; or any one of B19 nucleic acid, V9 nucleic acid, and A6 nucleic acid. In such embodiments, the sequence of the probe is selected such that it corresponds to a region that is the same between the different human EV genotypes to be detected.

In one embodiment, detection of target region VII nucleic acid involves production of an amplification product of at least 751, at least 725, at least 700, at least 675, at least 650, at least 625, at least 600, at least 575, at least 550, at least 525, at least 500, at least 475, at least 450, at least 425, at least 400, at least 375, at least 350, at least 325, at least 300, at least 275, at least 250, at least 225, at least 200, at least 175, at least 150, at least 125, at least 100, at least 90, at least 80, at least 70, at least 60, at least 50, at least 40, at least 30, at least 26, at least 24, at least 22, at least 20 consecutive nucleotides of SEQ ID NO:10.

The methods of the invention can involve detection of target region I nucleic acid either alone or in combination with detection of one or more of target regions I-VI and VIII as described herein. For example, detection of target region VII and detection of target region III; or detection of target region VII and detection of target region VIII; or detection of target region III and detection of target region VII; or detection of target region VII and detection of target region V; and the like. In addition, the methods of the invention can involve detection of 3 or more, or 4 or more, or 5 or more target regions, and the like. For example, the methods of the invention can involve detection of target region VII, target region II, target region III; or detection of target region III, target region VIII, target region VII; or detection of target region V, target region III, target region VII; and the like. It will be understood that detection of all combination of target regions I-VIII are contemplates by the present methods.

Exemplary primers and probes are discussed in greater detail below.

Target Region VIII

In another embodiment, the invention provides for detection of different human EV genotypes, e.g., B19, A6, and V9, in a sample, such as a biological sample, by detection of target nucleic acid sequence region VIII (FIG. 1, Target Region VIII, alignment position 4687-5137) as follows:

| | |
|---|---|
| TNCCACANAGTGGNCCAATTGGNGGTATTAAATCNATGGGAATNACTACNNTA | (SEQ ID NO: 11) |
| GTTCAGTATGCNGTGGGAATTATGACAGTNACNTATGACNTTTAAATTGGGNC | |
| CNCGNAANGCTACNGGNNGGTGGAATCCNCANCCTGGNGTNTATCCNCCNCA | |
| NGCAGCNGGTCATTTACCATATGTACTNTATGACCCNACAGCTACAGATGCAA | |
| ANCAACACCACAGNCANGGATANGAAAAGCCTGAAGAATTGTGGACNGCCAA | |
| AAGCCGTGTGCACCCATTGTAAACANTCCCCACCGTGNCCTCAGCCAGGANNC | |
| GTNACNNANCGNCCNCCNGTNCCNCCCAGANTNTANNTGCCCCCNCCNNTACC | |
| NNNNAGNNANCCNNNNNNAAAAGATANANANNNTGTAGAATNTAANTNNNT | |
| AACNNGNTATGAACAACATGTNATTAGAATG | |

or complement thereof, or a fragment thereof, wherein the variable nucleotide N is selected from a nucleotide at the corresponding position in either the B19 genome, A6 genome, or V9 genome as shown in the alignment of the three genotypes in FIG. 1.

Exemplary nucleic acid sequences suitable for design of primers for amplification of a Target Region VIII nucleic acid, and suitable for use in the methods of the invention, are indicated by bold typeface in FIG. 1. Suitable sequences for primers for amplification of Target Region VIII nucleic acid correspond to nucleotides 3-43, 66-82, 165-223, 234-312 and 427-450 of the 450 nucleotide sequence of SEQ ID NO:11 (FIG. 1), or a complement thereof.

Probes suitable for use in the invention can be designed from any sequence positioned within the sequence of an amplification product that would be produced using two selected primers. In some embodiments, the probe is selected to distinguish between the different human EV genotypes. In such embodiments the sequence of the probe is selected so that it corresponds to a region that differs in sequence by one or more nucleotides between the different human EV genotypes to be detected (e.g., the probe region can be selected so as to discriminate between B19, A6, and/or V9 genomes). Variations in the nucleotide sequences between the three genotypes are represented by circles surrounding the variable nucleotides in FIG. 1 and denoted by the variable N in SEQ ID NO:11.

In other embodiments, the probe is selected to detect any of the human EV genotypes which may or may not include discrimination between the human EV genotypes. Such probes can provide for detection of, for example, all three of B19 nucleic acid, V9 nucleic acid, and A6 nucleic acid; B19 nucleic acid and V9 nucleic acid; V9 nucleic acid and A6 nucleic acid; B19 nucleic acid, and A6 nucleic acid; or any one of B19 nucleic acid, V9 nucleic acid, and A6 nucleic acid. In such embodiments, the sequence of the probe is selected such that it corresponds to a region that is the same between the different human EV genotypes to be detected.

In one embodiment, detection of target region VIII nucleic acid involves production of an amplification product of at least 450, at least 425, at least 400, at least 375, at least 350, at least 325, at least 300, at least 275, at least 250, at least 225, at least 200, at least 175, at least 150, at least 125, at least 100, at least 90, at least 80, at least 70, at least 60, at least 50, at least 40, at least 30, at least 26, at least 24, at least 22, at least 20 consecutive nucleotides of SEQ ID NO:11.

The methods of the invention can involve detection of target region I nucleic acid either alone or in combination with detection of one or more of target regions I-VII as described herein. For example, detection of target region VIII and detection of target region III; or detection of target region VIII and detection of target region VIII; or detection of target region III and detection of target region VIII; or detection of target region VIII and detection of target region V; and the like. In addition, the methods of the invention can involve detection of 3 or more, or 4 or more, or 5 or more target regions, and the like. For example, the methods of the invention can involve detection of target region VIII, target region II, target region III; or detection of target region III, target region VIII, target region VII; or detection of target region V, target region III, target region VIII; and the like. It will be understood that detection of all combination of target regions I-VIII are contemplates by the present methods.

Exemplary primers and probes are discussed in greater detail below.

Primers And Probes

As described above, the target nucleic acid sequence regions I-VIII are conserved nucleic acid regions in different human EV genotypes, such as B19, A6, and V9. Primers and probes for use in these assays are preferably derived from the target nucleic acid sequence regions I-VIII as described above. In one embodiment of particular interest, primers and probes for use with the present assays are designed from the highly conserved nucleotide sequences of the target nucleic acid sequence regions I-VIII.

In general, the primers provide for amplification of target nucleic acid to produce as target nucleic acid amplification product (also referred to as an “amplicon”). Primers may be, and preferably are, used in connection with a probe. 5′ primers generally bind to a region to provide for amplification of the target nucleic, and preferably bind to a 5′ portion of the target sequence, as exemplified in FIG. 1. 3′ primers generally bind to a sequence that is complementary to a 3′ portion of the nucleic acid generated by extension from the 5′ primer, as exemplified in FIG. 1. The 5′ and 3′ primers may be separated by about 10, 20, 30, or 40 contiguous nucleotides, usually about 30 contiguous nucleotides. In certain embodiments, primers are designed so as to have a sequence complementary to one or more variant nucleotides within a target region sequence and/or to have a 3′ end adjacent a variant nucleotide of a sequence of a target region. Probes are generally designed so as to have a sequence complementary to one or more variant nucleotides within a target region sequence. In some embodiments involving amplification-based detection, probes are designed so as to have a sequence complementary to a sequence flanked by the sequence(s) complementary to one or more primers used for amplification.

Primers and probes for use in the assays herein are designed based on the sequence disclosed herein and are readily synthesized by standard techniques, e.g., solid phase synthesis via phosphoramidite chemistry, as disclosed in U.S. Pat. Nos. 4,458,066 and 4,415,732, incorporated herein by reference; Beaucage et al. (1992) Tetrahedron 48:2223-2311; and Applied Biosystems User Bulletin No. 13 (1 Apr. 1987). Other chemical synthesis methods include, for example, the phosphotriester method described by Narang et al., Meth. Enzymol. (1979) 68:90 and the phosphodiester method disclosed by Brown et al., Meth. Enzymol. (1979) 68:109. Poly(A) or poly(C), or other non-complementary nucleotide extensions may be incorporated into probes using these same methods. Hexaethylene oxide extensions may be coupled to probes by methods known in the art. Cload et al. (1991) J. Am. Chem. Soc. 113:6324-6326; U.S. Pat. No. 4,914,210 to Levenson et al.; Durand et al. (1990) Nucleic Acids Res. 18:6353-6359; and Horn et al. (1986) Tet. Lett. 27:4705-4708.

Typically, the primer sequences are in the range of between 10-75 nucleotides in length, such as 10 to 70, 12 to 65, 15 to 60, 20 to 55, 25 to 50, 30 to 45, and the like. More typically, primers are in the range of between 18 to 40, 19 to 35, 20 to 30, 21 to 29, 22 to 28, 23 to 27, 24-25 nucleotides long, and any length between the stated ranges. Primers of about 20 to 22 nucleotides in length are of particular interest.

The typical probe is in the range of between 10-50 nucleotides long, such as such as 10 to 50, 12 to 45, 15 to 40, 20 to 35, 25 to 30 and the like. More typically, probes are in the range of between 18 to 40, 19 to 35, 20 to 30, 21 to 29, 22 to 28, 23 to 27, 24-25 nucleotides long, and any length between the stated ranges. Probes of about 20 to 22 nucleotides in length are of particular interest.

In some embodiments, the subject methods provide for detection of any human EV genotype, e.g., B19, A6, or V9, in a sample, such a biological sample. In such embodiments, the subject methods detect a target nucleic acid region, or fragment thereof, by using primers and probe that correspond to sequences within the target region. Exemplary primers within the Target Regions I-VIII suitable for use in the methods of the invention are indicated by bold typeface in FIG. 1. Probes suitable for use in the invention can be designed from any sequence positioned within the sequence of an amplification product that would be produced using two selected primers. A probe suitable for use with such an embodiment is selected such that it corresponds to a region that shares a nucleotide sequence between the different human EV genotypes to be detected.

In other embodiments, the subject methods provide for detection and discrimination between different human EV genotypes, e.g., B19, A6, or V9, in a sample, such a biological sample. In such embodiments, the subject methods detect a target nucleic acid region, or fragment thereof, by using primers and probe that correspond to sequences within the target region. Exemplary primers within the Target Regions I-VIII suitable for use in the methods of the invention are indicated by bold typeface in FIG. 1. Probes suitable for use in the invention can be designed from any sequence positioned within the sequence of an amplification product that would be produced using two selected primers. In such embodiments the sequence of the probe is selected such that it corresponds to a region that differs in sequence by one or more nucleotides between the different human EV genotypes to be detected (e.g., the probe region can be selected so as to discriminate between B19, A6, and/or V9 genotypes). Variations in the nucleotide sequences between the three genotypes are represented in FIG. 1 by underlining the variable nucleotides.

Exemplary nucleic acid sequences of the human EV B19, A6, and V9 genotypes that are suitable for use are primers and probes in the assays of the present invention are described in Table 1 (human EV B19 genotype), Table 2 (human EV A6 genotype), and Table 3 (human EV V9 genotype). The sequence numbering presented in Tables 1-3 is the alignment numbering of FIG. 1, which corresponds to the numbering of the aligned genomes.

TABLE 1

Exemplary Primer and Probe Sequences for Detection of Target
Regions I-VIII of human EV B19, A6, and V9 Nucleic Acid (Se-
quence Provided Based on B19 Genome Sequence; Sequence Number-
ing Based on Alignment Numbering of FIG. 1)

| | | | | |
|---|---|---|---|---|
| SEQ ID NO.: | Start | End | Length | Sequence 5′ to 3′ |

Target Region I: 378-413

| | | | | |
|---|---|---|---|---|
| SEQ ID NO: 12 | 378 | 396 | 19 | AAATTTTAGCGGGCTTTTT |
| SEQ ID NO: 13 | 398 | 413 | 16 | CCCGCCTTATGCAAAT |

Target Region II: 583-856

| | | | | |
|---|---|---|---|---|
| SEQ ID NO: 14 | 583 | 600 | 18 | TAACAGGTATTTATACTA |
| SEQ ID NO: 15 | 611 | 629 | 19 | TACTAACATGGAGCTATTT |
| SEQ ID NO: 16 | 663 | 692 | 30 | GACTGTGCTAACGATAACTGGTGGTGCTCT |
| SEQ ID NO: 17 | 708 | 730 | 23 | ACTTCTGACTGGGAACCACTAAC |
| SEQ ID NO: 18 | 736 | 757 | 22 | CTAACAGACTAATGGCAATATA |
| SEQ ID NO: 19 | 831 | 856 | 26 | GAATGTAACAAATTTGAAGAAGGCTA |

Target Region III: 972-1114

| | | | | |
|---|---|---|---|---|
| SEQ ID NO: 20 | 972 | 994 | 23 | AAATTTTTGCCAGGAATGACTAC |
| SEQ ID NO: 21 | 1008 | 1036 | 29 | TTTAGAGATGGAGAGCAGTTTATAGAAAA |
| | 1042 | 1078 | 37 | TAATGAAAAAAATACCTTTAAATGTTGTAT |
| SEQ ID NO: 22 | | | | GGTGTGT |
| SEQ ID NO: 23 | 1095 | 1114 | 20 | TATATAGATACCTGTATTTC |

Target Region IV: 1258-1573

| | | | | |
|---|---|---|---|---|
| SEQ ID NO: 24 | 1258 | 1279 | 22 | TAAAGTTTCAAACTATGGTAAA |
| SEQ ID NO: 25 | 1374 | 1390 | 17 | TTTCAAATTCAAAGTGC |
| SEQ ID NO: 26 | 1453 | 1470 | 18 | CAGACTTTGAGCAGGTTA |
| SEQ ID NO: 27 | 1476 | 1492 | 17 | ATTAAAGACAATAAAAT |
| SEQ ID NO: 28 | 1553 | 1573 | 21 | GTGGATTGATAAAAAATGTGG |

Target Region V: 1605-2377

| | | | | | |
|---|---|---|---|---|---|
| SEQ ID NO: 29 | | 1605 | 1647 | 43 | CCAAGTACAGGAAAAACAAACTTGGCAAT |
| | | | | | GGCCATTGCTAAAA |
| SEQ ID NO: 30 | | 1674 | 1750 | 77 | TGGAATAATGAAAACTTTCCATTTAATGAT |
| | | | | | GTAGCAGGGAAAAGCTTGGTGGTCTGGGA |
| | | | | | TGAAGGTATTATTAAGTC |
| SEQ ID NO: 31 | EF2 | 1674 | 1708 | 35 | TGGAATAATGAAAACTTTCCATTTAATGAT |
| | | | | | GTAGC |
| SEQ ID NO: 32 | EP1a | 1720 | 1735 | 16 | TGGTGGTCTGGGATGA |
| SEQ ID NO: 33 | ER2a | 1762 | 1783 | 22 | TTCGACGTTTTCGGTAAAATCC |
| SEQ ID NO: 34 | | 1761 | 1783 | 23 | GAAGCTGCAAAAGCCATTTTAGG |
| SEQ ID NO: 35 | | 1794 | 1819 | 26 | ACCAGGGTAGATCAAAAAATGCGTGG |
| | | 1851 | 1882 | 32 | ATAACCAGCAATGGTGACATTACTTTTGTT |
| SEQ ID NO: 36 | | | | | GT |
| SEQ ID NO: 37 | | 1899 | 1924 | 26 | ACAACTGTACATGCTAAAGCCTTAAA |
| SEQ ID NO: 38 | EF6 | 1899 | 1924 | 26 | ACAACTGTaCATGCTAAAGCCTTAAA |
| SEQ ID NO: 39 | | 1932 | 1951 | 20 | ATGGTAAAGTTAAACTTTAC |
| SEQ ID NO: 40 | | 1954 | 1975 | 22 | TAAGATGCAGCCCTGACATGGG |
| SEQ ID NO: 41 | EP5 | 1962 | 1975 | 14 | AGCCCTGACATGGG |
| SEQ ID NO: 42 | | 1983 | 1999 | 17 | ACAGAGGCTGATGTACA |
| SEQ ID NO: 43 | | 2013 | 2033 | 21 | TGGTGTAATGCACAAAGCTGG |
| SEQ ID NO: 44 | ER4 | 2015 | 2033 | 19 | CACATTACGTGTTTCGACC |
| | | | | | (Complement of B19 sequence |
| | | | | | GTGTAATGCACAAAGCTGG |
| | | | | | SEQ ID NO: 45)) |
| SEQ ID NO: 46 | | 2036 | 2117 | 82 | CCACTATGAAAACTGGGCAATAAACTACA |
| | | | | | CTTTTGATTTCCCTGGAATTAATGCAGATG |
| | | | | | CCCTCCACCCAGACCTCCAAACC |
| SEQ ID NO: 47 | | 2124 | 2245 | 122 | ATTGTCACAGACACCAGTATCAGCAGCAG |
| | | | | | TGGTGGTGAAAGCTCTGAAGAACTCAGTG |
| | | | | | AAAGCAGCTTTTTTAACCTCATCACCCCAG |
| | | | | | GCGCCTGGAACACTGAAACCCCGCGCTCT |
| | | | | | AGTAC |
| SEQ ID NO: 48 | | 2251 | 2305 | 55 | TCCCCGGGACCAGTTCAGGAGAATCATTTG |
| | | | | | TCGGAAGCTCAGTTTCCTCCGAAGT |
| SEQ ID NO: 49 | EF17 | 2284 | 2305 | 22 | GAAGCTCAGTTTCCTCCGAAGT |
| SEQ ID NO: 50 | | 2352 | 2380 | 29 | CAGTTTCGTGAACTGTTAGTTGGGGTTGA |
| SEQ ID NO: 51 | EB2 | 2331 | 2350 | 20 | TTCTACACACCTTTGGCAGA |

Target Region VI: 2331-2608

| | | | | | |
|---|---|---|---|---|---|
| SEQ ID NO: 52 | ER13 | 2354 | 2377 | 24 | CAAAGCACTTGACAATCAACCCCA |
| | | | | | (Complement of B19 sequence |
| | | | | | GTTTCGTGAACTGTTAGTTGGGGT |
| | | | | | SEQ ID NO: 53) |
| SEQ ID NO: 54 | | 2484 | 2519 | 36 | TGTAGGAGCTTGGTATAATGGATGGAAATT |
| | | | | | TCGAGAATTTACC |
| SEQ ID NO: 55 | | 2583 | 2608 | 26 | AAAAAATGTGCTTACCTGTCTGGATTACAA |
| | | | | | AGCTTTGTAGATTATGAGTAAA |

Target Region VIII: 4687-5137

| | | | | |
|---|---|---|---|---|
| SEQ ID NO: 56 | 4690 | 4730 | 41 | CCACAAAGTGGGCCAATTGGAGGTATTAA |
| | | | | ATCAATGGGAAT |
| SEQ ID NO: 57 | 4753 | 4769 | 17 | GTGGGAATTATGACAGT |
| SEQ ID NO: 58 | 4852 | 4910 | 59 | GGTCATTTACCATATGTACTATATGACCCC |
| | | | | ACAGCTACAGATGCAAAACAACACCACAG |
| SEQ ID NO: 59 | 4921 | 4999 | 79 | GAAAAGCCTGAAGAATTGTGGACAGCCAA |
| | | | | AAGCCGTGTGCACCCATTGTAAACACTCCC |
| | | | | CACCGTGCCCTCAGCCAGGA |
| SEQ ID NO: 60 | 5114 | 5137 | 24 | TATGAACAACATGTTATTAGAATG |

TABLE 2

Exemplary Primer and Probe Sequences for Detection of Target Re-
gions I-VIII of human EV B19, A6, and V9 Nucleic Acid (Sequence
Provided Based on A6 Genome Sequence; Sequence Numbering Based on
Alignment Numbering of FIG. 1)

| | | | | |
|---|---|---|---|---|
| SEQ. ID NO.: | Start | Length | End | Sequence 5′ to 3′ |

Target Region I: 378-413

| | | | | |
|---|---|---|---|---|
| SEQ ID NO: 61 | 378 | 19 | 396 | AAATTTTCGCGGGCTTTTT |
| SEQ ID NO: 62 | 398 | 16 | 413 | CCCGCCTTATGCAAAT |

Target Region II: 583-856

| | | | | |
|---|---|---|---|---|
| SEQ ID NO: 63 | 583 | 18 | 600 | TAACAGGTATTTATACTA |
| SEQ ID NO: 64 | 611 | 19 | 629 | TACTAACATGGAGCTATTT |
| SEQ ID NO: 65 | 663 | 30 | 692 | GACTGTGCTAATGATAACTGGTGGTGCTCT |
| SEQ ID NO: 66 | 708 | 23 | 730 | ACTTCTGACTGGGAACCACTAAC |
| SEQ ID NO: 67 | 736 | 22 | 757 | CTAACAGACTAATGGCAATATA |
| SEQ ID NO: 68 | 831 | 26 | 856 | GAATGTAACAAATTTGAGGAAGGCTA |

Target Region III: 972-1114

| | | | | |
|---|---|---|---|---|
| SEQ ID NO: 69 | 972 | 23 | 994 | AAATTTTTGCCAGGAATGACTAC |
| SEQ ID NO: 70 | 1008 | 29 | 1036 | TTTAGAGATGGAGAGCAGTTTATAGAAAA |
| SEQ ID NO: 71 | 1042 | 37 | 1078 | TAATGAAAAAAATTCCTTTAAATGTTGTGTGGTGTGT |
| SEQ ID NO: 72 | 1095 | 20 | 1114 | TATATAGACACCTGTATTTC |

+TL,1L Target Region IV: 1258-1573

| | | | | |
|---|---|---|---|---|
| SEQ ID NO: 73 | 1258 | 22 | 1279 | TAAAGTTTCAAACAATGGTAAA |
| SEQ ID NO: 74 | 1374 | 17 | 1390 | TTTCAAATACAAAGTGC |
| SEQ ID NO: 75 | 1453 | 18 | 1470 | CAGACTTTGAGCAGGTTA |
| SEQ ID NO: 76 | 1476 | 17 | 1492 | ATTAAAGAAAATAAAAT |
| SEQ ID NO: 77 | 1553 | 21 | 1573 | GTGGATTGACAAAAAATGTGG |

Target Region V: 1605-2377

| | | | | |
|---|---|---|---|---|
| SEQ ID NO: 78 | 1605 | 43 | 1647 | CCAAGTACTGGAAAAACAAACTTGGCAATGGCTATT |
| | | | | GCTAAAA |
| SEQ ID NO: 79 | 1674 | 77 | 1750 | TGGAATAATGAAAATTTTCCATTTAATGATGTAGCGG |
| | | | | GGAAAAGTTTGGTGGTCTGGGATGAAGGCATTATTA |
| | | | | AGTC |
| SEQ ID NO: 80 | 1761 | 23 | 1783 | GAAGCTGCAAAAGCCATTTTAGG |
| SEQ ID NO: 81 | 1794 | 26 | 1819 | ACCAGGGTAGATCAAAAAATGCGTGG |
| SEQ ID NO: 82 | 1851 | 32 | 1882 | ATAACCAGCAATGGTGACATTACCTTTGTTGT |
| SEQ ID NO: 83 | 1899 | 26 | 1924 | ACAACTGTCCATGCTAAAGCCTTAAA |
| SEQ ID NO: 84 | 1932 | 20 | 1951 | ATGGTAAAGCTAAACTTTAC |
| SEQ ID NO: 85 | 1954 | 22 | 1975 | TAAGATGCAGCCCTGACATGGG |
| SEQ ID NO: 86 | 1983 | 17 | 1999 | ACAGAGGCTGATGTACA |
| SEQ ID NO: 87 | 2013 | 21 | 2033 | TGGTGTAATGCACAAAGCTGG |
| SEQ ID NO: 88 | 2036 | 82 | 2117 | CCACTATGAAAACTGGGCAATAAACTACACGTTTGAT |
| | | | | TTCCCTGGAATAAATGCAGATGCCCTCCACCCAGACC |
| | | | | TCCAAACC |
| SEQ ID NO: 89 | 2124 | 122 | 2245 | ATTGTCGCAGACACCAGTGTCAGCAGCAGTGGTGGT |
| | | | | GAAAGCTCTGAAGAACTCGGTGAAAGCAGCTTTTTC |
| | | | | AACCTCATCACTCCAGGCGCCTGGAACAGTGAAACC |
| | | | | CCGCGCTCTAGTAC |
| SEQ ID NO: 90 | 2332 | 20 | 2351 | TTTACACTCCACTTGCAGAC |
| (EA1) 10 | | | | |

Target Region VI: 2331-2608

| | | | | |
|---|---|---|---|---|
| SEQ ID NO: 91 | 2251 | 55 | 2305 | TCCCCGGGACCAGTTCAGGAGAATCATTTGTCGGAA |
| | | | | GCCCAGTTTCCTCCGAAGT |
| SEQ ID NO: 92 | 2352 | 29 | 2380 | CAGTTTCGTGAACTGTTAGTTGGGGTTGA |
| SEQ ID NO: 93 | 2477 | 20 | 2519 | TGTGGGAGCTTGGTATAATGGATGGAAGTTTCGTGAA |
| | | | | TTTACT |

Target Region VII: 2583-3333

| | | | | |
|---|---|---|---|---|
| SEQ ID NO: 94 | 2583 | 26 | 2608 | AAAAAATGTGCTTACTTGTCTGGATTACAAAGTTTTG |
| | | | | TGGATTATGAGTAAA |
| SEQ ID NO: 95 | 2715 | 30 | 2745 | TACTGAGACAGACTTAGAGCTTATTCAAATATTAAAA |
| | | | | GA |
| SEQ ID NO: 96 | 2764 | 17 | 2780 | ATTTCTTTAGATAATCCCTTAGA |
| SEQ ID NO: 97 | 2803 | 25 | 2827 | TTTGACTTAGTTGCTCGTATTAAAA |
| SEQ ID NO: 98 | 2843 | 28 | 2870 | CTCCAGACCTATATAGTCATCATTTTCA |
| SEQ ID NO: 99 | 2875 | 43 | 2917 | CATGGACAGTTATTTGACCACCCCCATGCCTTATCAC |
| | | | | CCAGTA |
| SEQ ID NO: 100 | 2927 | 65 | 2991 | CAGAACCTAGAGGAGAAGATGCAGTATTATCTAGTG |
| | | | | AAGACTTACACAAGCCTGGGCAAGTTAGC |
| SEQ ID NO: 101 | 2999 | 49 | 3047 | TACCCGGTACTAACTATATTGGCCCTGGCAATGAGCT |
| | | | | ACAAGCTGGGCC |
| SEQ ID NO: 102 | 3057 | 69 | 3125 | TGCTGTGGACAGTGCTGCAAGGATTCATGACTTTAGG |
| | | | | TATAGCCAATTGGCTAAGCTGGGAATAAATCC |
| SEQ ID NO: 103 | 3133 | 20 | 3152 | CATTGGACTGTAGCAGATGA |
| SEQ ID NO: 104 | 3160 | 113 | 3272 | TTAAAAAATATAAAAAATGAAACTGGGTTTCAAGCA |
| | | | | CAAGCAGTAAAAGATTACTTTACTTTAAAAGGTGCA |
| | | | | GCTGCCCCTGTGGCCCATTTTCAAGGAAGTTTGCCGG |
| | | | | AAGT |
| SEQ ID NO: 105 | 3280 | 17 | 3296 | TACAACGCCTCAGAAAA |
| SEQ ID NO: 106 | 3304 | 44 | 3347 | AGCATGACTTCAGTTAATTCTGCAGAAGCCAGCACTG |
| | | | | GTGCAGG |

Target Region VIII: 4687-5137

| | | | | |
|---|---|---|---|---|
| SEQ ID NO: 107 | 4690 | 41 | 4730 | CCACAGAGTGGACCAATTGGGGGTATTAAATCCATG |
| | | | | GGAAT |
| SEQ ID NO: 108 | 4753 | 17 | 4769 | GTGGGAATTATGACAGT |
| SEQ ID NO: 109 | 4852 | 59 | 4910 | GGTCATTTACCATATGTACTGTATGACCCTACAGCTA |
| | | | | CAGATGCAAAGCAACACCACAG |
| SEQ ID NO: 110 | 4921 | 79 | 4999 | GAAAAGCCTGAAGAATTGTGGACTGCCAAAAGCCGT |
| | | | | GTGCACCCATTGTAAACACTCCCCACCGTGTCCTCAG |
| | | | | CCAGGA |
| SEQ ID NO: 111 | 5114 | 24 | 5137 | TATGAACAACATGTAATTAGAATG |

TABLE 3

Exemplary Primer and Probe Sequences for Detection of Target
Regions I-VIII of human EV B19, A6, and V9 Nucleic Acid (Sequence
Provided Based on V9 Genome Sequence; Sequence Numbering Based on
Alignment Numbering of FIG. 1)

| | | | | |
|---|---|---|---|---|
| SEQ ID NO.: | Start | Length | End | Sequence 5′ to 3′ |

Target Region I: 378-413

| | | | | |
|---|---|---|---|---|
| SEQ ID NO: 112 | 378 | 19 | 396 | AAATTTTGGCGGGCTTTTT |
| SEQ ID NO: 113 | 398 | 16 | 413 | CCCGCCTTATGCAAAT |

Target Region II: 583-856

| | | | | |
|---|---|---|---|---|
| SEQ ID NO: 114 | 583 | 18 | 600 | TAACAGGTATTTATACTA |
| SEQ ID NO: 115 | 611 | 19 | 629 | TACTAACATGGAGCTATTT |
| SEQ ID NO: 116 | 663 | 30 | 692 | GACTGTGCTAATGATAACTGGTGGTGCTCT |
| SEQ ID NO: 117 | 708 | 23 | 730 | ACTTCTGACTGGGAACCACTAAC |
| SEQ ID NO: 118 | 736 | 22 | 757 | CTAACAGATTAATGGCAATATA |
| SEQ ID NO: 119 | 831 | 26 | 856 | GAATGTAACAAATTTGAGGAAGGCTA |

Target Region III: 972-1114

| | | | | |
|---|---|---|---|---|
| SEQ ID NO: 120 | 972 | 23 | 994 | AAATTTTTGCCAGGGATGACTAC |
| SEQ ID NO: 121 | 1008 | 29 | 1036 | TTTAGAGATGGAGAGCAGTTTATAGAAAA |
| SEQ ID NO: 122 | 1042 | 37 | 1078 | TAATGAAAAAAATTCCTTTAAATGTTGTGTGGTGTGT |
| SEQ ID NO: 123 | 1095 | 20 | 1114 | TATATAGACACCTGTATTTC |

Target Region IV: 1258-1573

| | | | | |
|---|---|---|---|---|
| SEQ ID NO: 124 | 1258 | 22 | 1279 | TAAAGTTTCAAACCATGGTAAA |
| SEQ ID NO: 125 | 1374 | 17 | 1390 | TTTCAAATTCAAAGTGC |
| SEQ ID NO: 126 | 1453 | 18 | 1470 | CAGACTTTGAGCAGGTTA |
| SEQ ID NO: 127 | 1476 | 17 | 1492 | ATTAAAGAAAATAAAAT |
| SEQ ID NO: 128 | 1553 | 21 | 1573 | GTGGATTGACAAAAAATGTGG |

Target Region V: 1605-2377

| | | | | |
|---|---|---|---|---|
| SEQ ID NO: 129 | 1605 | 43 | 1647 | CCAAGTACTGGAAAAACAAACTTGGCAATGGCTATT |
| | | | | GCTAAAA |
| SEQ ID NO: 130 | 1674 | 77 | 1750 | TGGAATAATGAAAACTTTCCATTTAATGATGTAGCGG |
| | | | | GGAAAAGTTTGGTGGTCTGGGATGAAGGCATTATTA |
| | | | | AGTC |
| SEQ ID NO: 131 | 1761 | 23 | 1783 | GAAGCTGCAAAAGCCATTTTAGG |
| SEQ ID NO: 132 | 1794 | 26 | 1819 | ACCAGGGTAGATCAGAAAATGCGTGG |
| SEQ ID NO: 133 | 1851 | 32 | 1882 | ATAACCAGCAATGGTGACATTACATTTGTTGT |
| SEQ ID NO: 134 | 1899 | 26 | 1924 | ACAACTGTGCATGCTAAAGCCTTAAA |
| SEQ ID NO: 135 | 1932 | 20 | 1951 | ATGGTAAAGCTAAACTTTAC |
| SEQ ID NO: 136 | 1954 | 22 | 1975 | TAAGATGTAGCCCTGACATGGG |
| SEQ ID NO: 137 | 1983 | 17 | 1999 | ACAGAGGCTGATGTACA |
| SEQ ID NO: 138 | 2013 | 21 | 2033 | TGGTGTAATGCACAAAGCTGG |
| SEQ ID NO: 139 | 2036 | 82 | 2117 | CCACTATGAAAACTGGGCAATAAACTACACATTTGAT |
| | | | | TTCCCTGGAATAAATGCAGATGCCCTCCACCCAGATC |
| | | | | TCCAAACC |
| SEQ ID NO: 140 | 2124 | 122 | 2245 | ATTGTCCCAGACACCAGTATCAGCAGCAGTGGTGGT |
| | | | | GAAAGCTCTGAAGAACTCAGTGAAAGCAGCTTTTTC |
| | | | | AACCTCATCACTCCAGGCGCCTGGAACAGTGAAACC |
| | | | | CCGCGCTCTAGTAC |
| SEQ ID NO: 141 | 2332 | 20 | 2351 | ATTTGCCCAGGACGTGTA |
| (EVD) | | | | |

Target Region VI: 2331-2608

| | | | | |
|---|---|---|---|---|
| SEQ ID NO: 142 | 2251 | 55 | 2305 | TCCCCGGGACCAGTTCAGGAGAATCATTTGTCGGAA |
| | | | | GCCCAGTTTCCTCCGAAGT |
| SEQ ID NO: 143 | 2352 | 29 | 2380 | CAGTTTCGTGAACTGTTAGTAGGGGTTGA |
| SEQ ID NO: 144 | 2477 | 20 | 2519 | TGTGGGAGCTTGGTATAATGGATGGAAAATTTAGAG |
| | | | | AGTTTACT |

Target Region VII: 2583-3333

| | | | | |
|---|---|---|---|---|
| SEQ ID NO: 145 | 2583 | 26 | 2608 | AAAAAATGTGCTTACCTGTCTGGATTACAAAGTTTTG |
| | | | | TGGATTATGAGTAAA |
| SEQ ID NO: 146 | 2600 | 34 | 2633 | GTCTGGATTACAAAGCTTTGTAGATTATGAGTAA |
| (EF31) | | | | |
| SEQ ID NO: 147 | 2764 | 32 | 2795 | TAAAGAAATCTATTAGGAAATCTCTTGGGGAG |
| (ER31) | | | | (Complement of B19 sequence |
| | | | | ATTTCTTTAGATAATCCCCTAGAAAACCCATC |
| | | | | SEQ ID NO: 148) |
| SEQ ID NO: 149 | 2667 | 18 | 2684 | ATTTGCCCAGGACGTGTA |
| (EVD) | | | | |
| SEQ ID NO: 150 | 2715 | 30 | 2745 | TACTGGAACAGACTTAGAGCTTATTCAAATTTTAAAA |
| | | | | GA |
| SEQ ID NO: 151 | 2764 | 17 | 2780 | ATTTCTTTAGATAATCCTTTAGA |
| SEQ ID NO: 152 | 2803 | 25 | 2827 | TTTGACTTAGTTGCTCGCATTAAAA |
| SEQ ID NO: 153 | 2843 | 28 | 2870 | CTCCAGACCTATATAGTCATCATTTTCA |
| SEQ ID NO: 154 | 2875 | 43 | 2917 | CATGGACAGTTATCTGACCACCCCCATGCCTTATCAT |
| | | | | CCAGTA |
| SEQ ID NO: 155 | 2927 | 65 | 2991 | CAGAACCTAGAGGAGAAAATGCAGTATTATCTAGTG |
| | | | | AAGACTTACACAAGCCTGGGCAAGTTAGC |
| SEQ ID NO: 156 | 2999 | 49 | 3047 | TACCCGGTACTAACTATGTTGGGCCTGGCAATGAGCT |
| | | | | ACAAGCTGGGCC |
| SEQ ID NO: 157 | 3057 | 69 | 3125 | TGCTGTGGACAGTGCTGCAAGGATTCATGACTTTAGG |
| | | | | TATAGCCAATTGGCTAAGTTGGGAATAAATCC |
| SEQ ID NO: 158 | 3133 | 20 | 3152 | CATTGGACGGTAGCAGATGA |
| SEQ ID NO: 159 | 3160 | 113 | 3272 | TTAAAAAATATAAAAAATGAAACAGGGTTTCAAGCA |
| | | | | CAAGCAGTAAAAGATTACTTTACTTTAAAAGGTGCA |
| | | | | GCTGCCCCTGTGGCCCATTTTCAAGGAAGTTTACCGG |
| | | | | AAGT |
| SEQ ID NO: 160 | 3280 | 17 | 3296 | TACAACGCCTCAGAAAA |
| SEQ ID NO: 161 | 3304 | 44 | 3347 | AGCATGACTTCAGTTAACTCTGCAGAAGCCAGCACTG |
| | | | | GTGCAGG |

Target Region VIII: 4687-5137

| | | | | |
|---|---|---|---|---|
| SEQ ID NO: 162 | 4690 | 41 | 4730 | CCACAAAGTGGGCCAATTGGAGGTATTAAATCCATG |
| | | | | GGAAT |
| SEQ ID NO: 163 | 4753 | 17 | 4769 | GTGGGAATTATGACAGT |
| SEQ ID NO: 164 | 4852 | 59 | 4910 | GGTCATTTACCATATGTACTGTATGACCCCACAGCTA |
| | | | | CAGATGCAAAGCAACACCACAG |
| SEQ ID NO: 165 | 4921 | 79 | 4999 | GAAAAGCCTGAAGAATTGTGGACTGCCAAAAGCCGT |
| | | | | GTGCACCCATTGTAAACATTCCCCACCGTGTCCTCAG |
| | | | | CCAGGA |
| SEQ ID NO: 166 | 5114 | 24 | 5137 | TATGAACAACATGTAATTAGAATG |

The probes may be coupled to labels for detection. There are several methods and compositions known for derivatizing oligonucleotides with reactive functionalities which permit the addition of a label. For example, several approaches are available for biotinylating probes so that radioactive, fluorescent, chemiluminescent, enzymatic, or electron dense labels can be attached via avidin. See, e.g., Broken et al., Nucl. Acids Res. (1978) 5:363-384 which discloses the use of ferritin-avidin-biotin labels; and Chollet et al. Nucl. Acids Res. (1985) 13:1529-1541 which discloses biotinylation of the 5′ termini of oligonucleotides via an aminoalkylphosphoramide linker arm. Several methods are also available for synthesizing amino-derivatized oligonucleotides which are readily labeled by fluorescent or other types of compounds derivatized by amino-reactive groups, such as isothiocyanate, N-hydroxysuccinimide, or the like, see, e.g., Connolly (1987) Nucl. Acids Res. 15:3131-3139, Gibson et al. (1987) Nucl. Acids Res. 15:6455-6467 and U.S. Pat. No. 4,605,735 to Miyoshi et al. Methods are also available for synthesizing sulfhydryl-derivatized oligonucleotides which can be reacted with thiol-specific labels, see, e.g., U.S. Pat. No. 4,757,141 to Fung et al., Connolly et al. (1985) Nuc. Acids Res. 13:4485-4502 and Spoat et al. (1987) Nucl. Acids Res. 15:4837-4848. A comprehensive review of methodologies for labeling DNA fragments is provided in Matthews et al., Anal. Biochem. (1988) 169:1-25.

For example, probes may be fluorescently labeled by linking a fluorescent molecule to the non-ligating terminus of the probe. Guidance for selecting appropriate fluorescent labels can be found in Smith et al., Meth. Enzymol. (1987) 155:260-301; Karger et al., Nucl. Acids Res. (1991) 19:4955-4962; Haugland (1989) Handbook of Fluorescent Probes and Research Chemicals (Molecular Probes, Inc., Eugene, Oreg.). Preferred fluorescent labels include fluorescein and derivatives thereof, such as disclosed in U.S. Pat. No. 4,318,846 and Lee et al., Cytometry (1989) 10:151-164, and 6-FAM, JOE, TAMRA, ROX, HEX-1, HEX-2, ZOE, TET-1 or NAN-2, and the like.

Additionally, probes can be labeled with an acridinium ester (AE). Current technologies allow the AE label to be placed at any location within the probe. See, e.g., Nelson et al. (1995) “Detection of Acridinium Esters by Chemiluminescence” in Nonisotopic Probing, Blotting and Sequencing, Kricka L. J. (ed) Academic Press, San Diego, Calif.; Nelson et al. (1994) “Application of the Hybridization Protection Assay (HPA) to PCR” in The Polymerase Chain Reaction, Mullis et al. (eds.) Birkhauser, Boston, Mass.; Weeks et al., Clin. Chem. (1983) 29:1474-1479; Berry et al., Clin. Chem. (1988) 34:2087-2090. An AE molecule can be directly attached to the probe using non-nucleotide-based linker arm chemistry that allows placement of the label at any location within the probe. See, e.g., U.S. Pat. Nos. 5,585,481 and 5,185,439.

If a solid support is used in the assay (e.g., to capture amplicons of target nucleic acid using a probe), the oligonucleotide probe may be attached to the solid support in a variety of manners. For example, the probe may be attached to the solid support by attachment of the 3′ or 5′ terminal nucleotide of the probe to the solid support. More preferably, the probe is attached to the solid support by a linker which senses to distance the probe from the solid support. The linker is usually at least 15-30 atoms in length, more preferably at least 15-50 atoms in length. The required length of the linker will depend on the particular solid support used. For example, a six atom linker is generally sufficient when high cross-linked polystyrene is used as the solid support.

A wide variety of linkers are known in the art which may be used to attach the oligonucleotide probe to the solid support. The linker may be formed of any compound which does not significantly interfere with the hybridization of the target sequence to the probe attached to the solid support. The linker may be formed of a homopolymeric oligonucleotide which can be readily added on to the linker by automated synthesis. Alternatively, polymers such as functionalized polyethylene glycol can be used as the linker. Such polymers are preferred over homopolymeric oligonucleotides because they do not significantly interfere with the hybridization of probe to the target oligonucleotide. Polyethylene glycol is particularly preferred.

The linkages between the solid support, the linker and the probe are normally not cleaved during removal of base protecting groups under basic conditions at high temperature. Examples of preferred linkages include carbamate and amide linkages.

Examples of preferred types of solid supports for immobilization of the oligonucleotide probe include controlled pore glass, glass plates, polystyrene, avidin-coated polystyrene beads, cellulose, nylon, acrylamide gel and activated dextran.

In certain embodiments, an internal control (IC) or an internal standard is added to serve as a control to show that any negative result is not due to failure of the assay. The use of the IC permits the control of the separation process, the amplification process, and the detection system, and permits the monitoring of assay performance and quantification for the sample(s). The IC can be included at any suitable point, for example, in the lysis buffer. In one embodiment, the IC comprises phage nucleic acid. Where a solid support is used in the assay, the solid support may additionally include probes specific to the internal standard (IC probe), thereby facilitating capture when using the IC probe. The IC probe can optionally be coupled with a detectable label that is different from the detectable label for the target sequence. In embodiments where the detectable label is a fluorophore, the IC can be quantified spectrophotometrically and by limit of detection studies.

In another embodiment, an IC, as described herein, is combined with RNA isolated from the sample according to standard techniques known to those of skill in the art, and described herein. The RNA is then reverse-transcribed using a reverse transcriptase to provide copy DNA. The cDNA sequences can be optionally amplified (e.g., by PCR) using labeled primers. The amplification products are separated, typically by electrophoresis, and the amount of radioactivity (proportional to the amount of amplified product) is determined. The amount of mRNA in the sample can then calculated where desired by comparison with the signal produced by the known standards.

Detection of Human EV in a Sample

In one aspect, the assay detects the presence of human EV in a sample. In such an aspect, the assay is an amplification-based assay using degenerate primers and probes, where the primers and probes are designed to provide for amplification of a target nucleic acid sequence region of the human EV genome.

As discussed above, the assay detects the presence of one or more target nucleic acid regions (e.g., Target Regions I-VIII), or a portion thereof. The target nucleic acid sequence regions I-VIII are conserved nucleic acid regions in different human EV genotypes, such as B19, A6, and V9. Primers and probes for use in these assays are preferably derived from the target nucleic acid sequence regions I-VIII as described above. Particularly preferred primers and probes for use with the present assays are designed from the highly conserved nucleotide sequences of the target nucleic acid sequence regions I-VIII.

As discussed above, in one embodiment, the primers and/or probes are designed for nucleic acid-based detection, particularly an amplification method, of a target nucleic acid having a target nucleic acid sequence described above, e.g., target nucleic acid sequence region I-VIII. That is, in such an embodiment, the primers are designed to amplify a target sequence having the nucleic acid sequence of a nucleic acid sequence described above, e.g., target nucleic acid sequence region I-VIII.

In another embodiment, the primers and/or probes are designed for nucleic acid-based detection, particularly an amplification method, of a target nucleic acid having a nucleic acid sequence that is a fragment of a target nucleic acid sequence described above, e.g., target nucleic acid sequence region I-VIII. That is, in such an embodiment, the primers are designed to amplify a target sequence having the nucleic acid sequence of a portion smaller than the entire nucleic acid sequence described above, e.g., target nucleic acid sequence region I-VIII.

Specific detection of human EV nucleic acid in a sample is generally accomplished by detection of one or more of the target sequence regions I-VIII, or a fragment thereof. In one embodiment, human EV target nucleic acid is detected by use of primers and probes designed upon the sequences of target sequence region V.

In an embodiment of particular interest, the target sequence is detected using primers having the sequence TGGAATAATGAAAACTTTCCATTTAATGATGTAGC (5′ primer) (SEQ ID NO:31) [EF2], TTCGACGTTTTCGGTAAAATCC (3′ primer) (SEQ ID NO:33) [ER2a], and a probe having the sequence TGGTGGTCTGGGATGA (SEQ ID NO:32) [EP1a] is of particular interest.

In another embodiment of particular interest, the target sequence is detected using primers having the sequence ACAACTGTaCATGCTAAAGCCTTAAA (5′ primer) (SEQ ID NO:38) [EF6] and CACATTACGTGTTTCGACC (3′ primer) (SEQ ID NO:44) ER4], and a probe having the sequence AGCCCTGACATGGG (SEQ ID NO:41) [EP5] is of particular interest.

Of particular interest is the use of these primers and probes in a real-time RT PCR method for detection of human EV in a sample, with use of a dual-labeled TaqMan Probe.

Discrimination Between Different Human EV Genotypes in a Sample

In another aspect, the subject assay discriminates between different genotypes of human EV, such as B19, A6, and V9, in a sample. In such an aspect, the assay is an amplification-based assay using degenerate primers and genotype (e.g., B19, A6, or V9) specific probes, where the primers are designed to provide for amplification of a target nucleic acid sequence region of human EV, and the genotype specific probes are designed to detect the amplification of a target nucleic acid sequence region of a particular human EV genotype (e.g., B19, A6, or V9).

As discussed above, the assay detects the presence of one or more target nucleic acid regions (e.g., Target Regions I-VIII), or a portion thereof. The target nucleic acid sequence regions I-VIII are conserved nucleic acid regions in different human EV genotypes, such as B19, A6, and V9. Primers and probes for use in these assays are preferably derived from the target nucleic acid sequence regions I-VIII as described above.

Probes suitable for use in the invention can be designed from any sequence positioned within the sequence of an amplification product that would be produced using two selected primers. In such embodiments the sequence of the probe is selected such that it corresponds to a region that differs in sequence by one or more nucleotides between the different human EV genotypes to be detected (e.g., the probe region can be selected so as to discriminate between B19, A6, and/or V9 genotypes).

As discussed above, in one embodiment, the primers and/or probes are designed for nucleic acid-based detection, particularly an amplification method, of a target nucleic acid having a target nucleic acid sequence described above, e.g., target nucleic acid sequence region I-VIII. That is, in such an embodiment, the primers are designed to amplify a target sequence having the nucleic acid sequence of a nucleic acid sequence described above, e.g., target nucleic acid sequence region I-VIII.

In another embodiment, the primers and/or probes are designed for nucleic acid-based detection, particularly an amplification method, of a target nucleic acid having a nucleic acid sequence that is a fragment of a target nucleic acid sequence described above, e.g., target nucleic acid sequence region I-VIII. That is, in such an embodiment, the primers are designed to amplify a target sequence having the nucleic acid sequence of a portion smaller than the entire nucleic acid sequence described above, e.g., target nucleic acid sequence region I-VIII.

Specific detection and discrimination of the nucleic acid of a particular genotype of human EV, such as B19, A6, or V9 in a sample is generally accomplished by detection of one of the target sequence regions I-VIII, or fragments thereof.

In one embodiment, detection and discrimination of particular genotypes of human EV, such as B19, A6, and V9 is by use of primers and probes designed upon the sequences of target nucleic acid sequence regions V and VII.

In an embodiment of particular interest, the target nucleic acid sequence region V is detected using primers having the sequence GAABCTCAGTTTCCTCCGAAGT (5′ primer) (SEQ ID NO:49) [EF17] and CAAAGCACTTGACAATCAACCCCA (3′ primer) (SEQ ID NO:54), [ER13] a B19 genotype specific probe having the sequence TTCTACACACCTTTGGCAGA (SEQ ID NO:51) [EB2], and a A6 genotype specific probe having the sequence TTTACACTCCACTTGCAGAC (SEQ ID NO:90) [EA1], is of particular interest.

In an embodiment of particular interest, the target nucleic acid sequence region VII is detected using primers having the sequence GTCTGGATTACAAAGCTTTGTAGATTATGAGTAA (5′ primer) (SEQ ID NO:146) [EF31] and TAAAGAAATCTATTAGGAAATCTCTTGGGGAG (3′ primer) (SEQ ID NO: 147), [ER31] and a V9 genotype specific probe having the sequence ATTTGCCCAGGACGTGTA (SEQ ID NO:141) [EVD] is of particular interest.

Of particular interest is the use of these primers and probes in a real-time RT PCR method for detection of human EV, with use of a dual-labeled TaqMan Probe.

Methods of Detection

The invention provides DNA-based assay for detecting human EV in a sample. In preferred embodiments, the methods discriminate between different genotypes of human EV, such as B19, A6 and V9. Detection may be done using a wide variety of methods, including direct sequencing, hybridization with sequence-specific oligomers, gel electrophoresis and mass spectrometry. These methods can use heterogeneous or homogeneous formats, isotopic or nonisotopic labels, as well as no labels at alt.

Preferably, the methods involve amplifying nucleic acids from a sample. If a diagnostic nucleic acid is obtained, the presence of human EV in a sample is indicated. In general, the methods involve amplifying a nucleic acid from a sample using a detection primer and at least one other primer, as described above, and assessing the amplified nucleic acids. The methods are highly sensitive, and may detect as few as 1.5 copies of human EV per reaction, which is equivalent to 75 copies of DNA per mL of specimen, although detection may be limited by the limit of linear range detection, which ends 1.5×10⁶ copies of DNA per reaction. Thus, the invention generally provides for detection of human EV in a sample, where the human EV is present in at least 75 copies of DNA per mL of specimen.

As is known in the art, an amplified nucleic acid may be assessed by a number of methods, including, for example, determining the presence or absence of the nucleic acid, determining the size of the nucleic acid or determining the abundance of a nucleic acid in relation to another amplified nucleic acid. In most embodiments, an amplified nucleic acid is assessed using gel electrophoresis, nucleic acid hybridization, sequencing, and/or detection of a signal from a label bound to the amplified nucleic acid. Methods of amplifying (e.g., by polymerase chain reaction) nucleic acid, methods of performing primers extension, and methods of assessing nucleic acids are generally well known in the art (e.g., see Ausubel, et al, Short Protocols in Molecular Biology, 3rd ed., Wiley & Sons, 1995 and Sambrook, et al, Molecular Cloning: A Laboratory Manual, Third Edition, (2001) Cold Spring Harbor, N.Y.) and need not be described in any great detail.

For example, primers and probes described above may be used in polymerase chain reaction (PCR)-based techniques to detect human EV in biological samples. PCR is a technique for amplifying a desired target nucleic acid sequence contained in a nucleic acid molecule or mixture of molecules. In PCR, a pair of primers is employed in excess to hybridize to the complementary strands of the target nucleic acid. The primers are each extended by a polymerase using the target nucleic acid as a template. The extension products become target sequences themselves after dissociation from the original target strand. New primers are then hybridized and extended by a polymerase, and the cycle is repeated to geometrically increase the number of target sequence molecules. The PCR method for amplifying target nucleic acid sequences in a sample is well known in the art and has been described in, e.g., Innis et al. (eds.) PCR Protocols (Academic Press, NY 1990); Taylor (1991) Polymerase chain reaction: basic principles and automation, in PCR: A Practical Approach, McPherson et al. (eds.) IRL Press, Oxford; Saiki et al. (1986) Nature 324:163; as well as in U.S. Pat. Nos. 4,683,195, 4,683,202 and 4,889,818, all incorporated herein by reference in their entireties.

In particular, PCR uses relatively short oligonucleotide primers which flank the target nucleotide sequence to be amplified, oriented such that their 3′ ends face each other, each primer extending toward the other. The polynucleotide sample is extracted and denatured, preferably by heat, and hybridized with first and second primers which are present in molar excess. Polymerization is catalyzed in the presence of the four deoxyribonucleotide triphosphates (dNTPs—dATP, dGTP, dCTP and dTTP) using a primer- and template-dependent polynucleotide polymerizing agent, such as any enzyme capable of producing primer extension products, for example, E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). This results in two “long products” which contain the respective primers at their 5′ ends covalently linked to the newly synthesized complements of the original strands.

The reaction mixture is then returned to polymerizing conditions, e.g., by lowering the temperature, inactivating a denaturing agent, or adding more polymerase, and a second cycle is initiated. The second cycle provides the two original strands, the two long products from the first cycle, two new long products replicated from the original strands, and two “short products” replicated from the long products. The short products have the sequence of the target sequence with a primer at each end. On each additional cycle, an additional two long products are produced, and a number of short products equal to the number of long and short products remaining at the end of the previous cycle. Thus, the number of short products containing the target sequence grow exponentially with each cycle. Preferably, PCR is carried out with a commercially available thermal cycler, e.g., Perkin Elmer.

The fluorogenic 5′ nuclease assay, known as the TAQMAN™ assay (Perkin-Elmer), is a powerful and versatile PCR-based detection system for nucleic acid targets. For a detailed description of the TAQMAN™ assay, reagents and conditions for use therein, see, e.g., Holland et al., Proc. Natl. Acad. Sci, U.S.A. (1991) 88:7276-7280; U.S. Pat. Nos. 5,538,848, 5,723,591, and 5,876,930, all incorporated herein by reference in their entireties. Hence, primers and probes derived from regions of the human EV genome described herein can be used in TAQMAN™ analyses to detect the presence of infection in a biological sample. Analysis is performed in conjunction with thermal cycling by monitoring the generation of fluorescence signals. The assay system dispenses with the need for gel electrophoretic analysis, and has the capability to generate quantitative data allowing the determination of target copy numbers.

The fluorogenic 5′ nuclease assay is conveniently performed using, for example, AMPLITAQ GOLD™ DNA polymerase, which has endogenous 5′ nuclease activity, to digest an internal oligonucleotide probe labeled with both a fluorescent reporter dye and a quencher (see, Holland et al., Proc. Natl. Acad. Sci. USA (1991) 88:7276-7280; and Lee et al., Nucl. Acids Res. (1993) 21:3761-3766). Assay results are detected by measuring changes in fluorescence that occur during the amplification cycle as the fluorescent probe is digested, uncoupling the dye and quencher labels and causing an increase in the fluorescent signal that is proportional to the amplification of target nucleic acid.

The amplification products can be detected in solution or using solid supports. In this method, the TAQMAN™ probe is designed to hybridize to a target sequence within the desired PCR product. The 5′ end of the TAQMAN™ probe contains a fluorescent reporter dye. The 3′ end of the probe is blocked to prevent probe extension and contains a dye that will quench the fluorescence of the 5′ fluorophore. During subsequent amplification, the 5′ fluorescent label is cleaved off if a polymerase with 5′ exonuclease activity is present in the reaction. Excision of the 5′ fluorophore results in an increase in fluorescence which can be detected.

In particular, the oligonucleotide probe is constructed such that the probe exists in at least one single-stranded conformation when unhybridized where the quencher molecule is near enough to the reporter molecule to quench the fluorescence of the reporter molecule. The oligonucleotide probe also exists in at least one conformation when hybridized to a target polynucleotide such that the quencher molecule is not positioned close enough to the reporter molecule to quench the fluorescence of the reporter molecule. By adopting these hybridized and unhybridized conformations, the reporter molecule and quencher molecule on the probe exhibit different fluorescence signal intensities when the probe is hybridized and unhybridized. As a result, it is possible to determine whether the probe is hybridized or unhybridized based on a change in the fluorescence intensity of the reporter molecule, the quencher molecule, or a combination thereof. In addition, because the probe can be designed such that the quencher molecule quenches the reporter molecule when the probe is not hybridized, the probe can be designed such that the reporter molecule exhibits limited fluorescence unless the probe is either hybridized or digested.

Accordingly, the present invention relates to methods for amplifying a target human EV nucleotide sequence using a nucleic acid polymerase having 5′ to 3′ nuclease activity, one or more primers capable of hybridizing to the target human EV sequence or its extension product, and an oligonucleotide probe capable of hybridizing to the target human EV sequence 3′ relative to the primer. During amplification, the polymerase digests the oligonucleotide probe when it is hybridized to the target sequence, thereby separating the reporter molecule from the quencher molecule. As the amplification is conducted, the fluorescence of the reporter molecule is monitored, with fluorescence corresponding to the occurrence of nucleic acid amplification. The reporter molecule is preferably a fluorescein dye and the quencher molecule is preferably a rhodamine dye.

Another method of detection involves use of target sequence-specific oligonucleotide probes, which contain a region of complementarity to the target sequence described above. The probes may be used in hybridization protection assays (HPA). In this embodiment, the probes are conveniently labeled with acridinium ester (AE), a highly chemiluminescent molecule. See, e.g., Nelson et al. (1995) “Detection of Acridinium Esters by Chemiluminescence” in Nonisotopic Probing, Blotting and Sequencing, Kricka L. J. (ed) Academic Press, San Diego, Calif.; Nelson et al. (1994) “Application of the Hybridization Protection Assay (HPA) to PCR” in The Polymerase Chain Reaction, Mullis et al. (eds.) Birkhauser, Boston, Mass.; Weeks et al., Clin. Chem. (1983) 29:1474-1479; Berry et al., Clin. Chem. (1988) 34:2087-2090. One AE molecule is directly attached to the probe using a non-nucleotide-based linker arm chemistry that allows placement of the label at any location within the probe. See, e.g., U.S. Pat. Nos. 5,585,481 and 5,185,439. Chemiluminescence is triggered by reaction with alkaline hydrogen peroxide which yields an excited N-methyl acridone that subsequently collapses to ground state with the emission of a photon. Additionally, AE causes ester hydrolysis which yields the nonchemiluminescent-methyl acridinium carboxylic acid.

When the AE molecule is covalently attached to a nucleic acid probe, hydrolysis is rapid under mildly alkaline conditions. When the AE-labeled probe is exactly complementary to the target nucleic acid, the rate of AE hydrolysis is greatly reduced. Thus, hybridized and unhybridized AE-labeled probe can be detected directly in solution, without the need for physical separation.

HPA generally consists of the following steps: (a) the AE-labeled probe is hybridized with the target nucleic acid in solution for about 15 to about 30 minutes. A mild alkaline solution is then added and AE coupled to the unhybridized probe is hydrolyzed. This reaction takes approximately 5 to 10 minutes. The remaining hybrid-associated AE is detected as a measure of the amount of target present. This step takes approximately 2 to 5 seconds. Preferably, the differential hydrolysis step is conducted at the same temperature as the hybridization step, typically at 50 to 70 degrees celsius. Alternatively, a second differential hydrolysis step may be conducted at room temperature. This allows elevated pHs to be used, for example in the range of 10-11, which yields larger differences in the rate of hydrolysis between hybridized and unhybridized AE-labeled probe. HPA is described in detail in, e.g., U.S. Pat. Nos. 6,004,745; 5,948,899; and 5,283,174, the disclosures of which are incorporated by reference herein in their entireties.

The oligonucleotide molecules of the present invention may also be used in nucleic acid sequence-based amplification (NASBA). This method is a promoter-directed, enzymatic process that induces in vitro continuous, homogeneous and isothermal amplification of a specific nucleic acid to provide RNA copies of the nucleic acid. The reagents for conducting NASBA include a first DNA primer with a 5′ tail comprising a promoter, a second DNA primer, reverse transcriptase, RNAse-H, T7 RNA polymerase, NTP's and dNTP's. Using NASBA, large amounts of single-stranded RNA are generated from either single-stranded RNA or DNA, or double-stranded DNA. When RNA is to be amplified, the ssRNA serves as a template for the synthesis of a first DNA strand by elongation of a first primer containing an RNA polymerase recognition site. This DNA strand in turn serves as the template for the synthesis of a second, complementary, DNA strand by elongation of a second primer, resulting in a double-stranded active RNA-polymerase promoter site, and the second DNA strand serves as a template for the synthesis of large amounts of the first template, the ssRNA, with the aid of a RNA polymerase. The NASBA technique is known in the art and described in, e.g., European Patent 329,822, International Patent Application No. WO 91/02814, and U.S. Pat. Nos. 6,063,603, 5,554,517 and 5,409,818, all of which are incorporated herein in their entireties.

The human EV sequences described herein are also useful in nucleic acid hybridization and amplification techniques that utilize branched DNA molecules. In a basic nucleic acid hybridization assay, single-stranded analyte nucleic acid is hybridized to a labeled single-stranded nucleic acid probe and resulting labeled duplexes are detected. Variations of this basic scheme have been developed to facilitate separation of the duplexes to be detected from extraneous materials and/or to amplify the signal that is detected. One method for amplifying the signal uses amplification multimers that are polynucleotides with a first segment that hybridizes specifically to the analyte nucleic acid or a strand of nucleic acid bound to the analyte and iterations of a second segment that hybridizes specifically to a labeled probe. The amplification is theoretically proportional to the number of iterations of the second segment. The multimers may be either linear or branched. Two general types of branched multimers are useful in these techniques: forked and combed. Methods for making and using branched nucleic acid molecules are known in the art and described in, e.g., U.S. Pat. No. 5,849,481, incorporated herein by reference in its entirety.

As is readily apparent, design of the assays described herein are subject to a great deal of variation, and many formats are known in the art. The above descriptions are merely provided as guidance and one of skill in the art can readily modify the described protocols, using techniques well known in the art.

Kits

Kits for use in connection with the subject invention are also provided. The above-described assay reagents, including the primers, probes, solid support with bound probes, as well as other detection reagents, can be provided in kits, with suitable instructions and other necessary reagents, in order to conduct the assays as described above. The kit will normally contain in separate containers the combination of primers and probes (either already bound to a solid matrix or separate with reagents for binding them to the matrix), control formulations (positive and/or negative), labeled reagents when the assay format requires same and signal generating reagents (e.g., enzyme substrate) if the label does not generate a signal directly. Instructions (e.g., written, tape, VCR, CD-ROM, etc.) for carrying out the assay usually will be included in the kit. The kit can also contain, depending on the particular assay used, other packaged reagents and materials (i.e. wash buffers and the like). Standard assays, such as those described above, can be conducted using these kits.

The instructions are generally recorded on a suitable recording medium. For example, the instructions may be printed on a substrate, such as paper or plastic, etc. As such, the instructions may be present in the kits as a package insert, in the labeling of the container of the kit or components thereof (e.g., associated with the packaging or subpackaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed from or from where the instructions can be downloaded.

Still further, the kit may be one in which the instructions are obtained are downloaded from a remote source, as in the Internet or world wide web. Some form of access security or identification protocol may be used to limit access to those entitled to use the subject invention. As with the instructions, the means for obtaining the instructions and/or programming is generally recorded on a suitable recording medium.

In general, kits of the invention include at least one primer, usually at least two primers (a 5′ and a 3′ primer), usually at least two primers and a probe, as described above. Kits may also contain instructions for using the kit to detect human EV in a sample using the methods described above, including the above discussed PCR methods. Also included in the subject kits may be buffers, dNTPs, and controls, (e.g., positive and negative control nucleic acids) for performing the subject methods. Primers in the subject kits may be detectably labeled or unlabeled).

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Materials and Methods

The following method and material were used in the Example(s) below.

Specimen Types and Handing. Samples for use in detection of Erythrovirus according to the invention can be any suitable biological sample, such as serum, plasma, amniotic fluid, and tissue specimen. Tissue specimens should be stored frozen at −20±10° C. in saline or phosphate buffered saline (PBS). Serum, plasma, and amniotic fluid should be stored frozen at −20±10° C. All of the above specimen types, as needed, can be shipped on dry ice via overnight express,

Primers and Probes. Oligonucleotide primers and fluorogenic probes were synthesized by qualified vendors. Oligonucleotide primers were desalted and lyophilized. Oligonucleotide primer pair sets for detection of Erythrovirus (22.5 μM working concentration) and corresponding fluorogenic probe (10 μM working concentration) were as follows:

Erythrovirus Primer/Probe Set 1

(SEQ ID NO: 31)

| | |
|---|---|
| EF2 | 5′-TGGAATAATGAAAACTTTCCATTTAATGATGTAGC-3′ |

(SEQ ID NO: 33)

| | |
|---|---|
| ER2a | 5′-CCTAAAATGGCTTTTGCAGCTT-3′ |

(SEQ ID NO: 32)

| | |
|---|---|
| EP1a | 5′-TGGTGGTCTGGGATGA-3′ |
| (FAM-la- | |
| beled MGB | |
| probe) | |

Erythrovirus Primer/Probe Set 2

(SEQ ID NO: 38)

| | |
|---|---|
| EF6 | 5′-ACAACTGTaCATGCTAAAGCCTTAAA-3′ |

(SEQ ID NO: 44)

| | |
|---|---|
| ER4 | 5′-CCAGCTTTGTGCATTACAC-3′ |

(SEQ ID NO: 41)

| | |
|---|---|
| EP5 | 5′-AGCCCTGACATGGG-3′ |
| (FAM-la- | |
| beled MGB | |
| probe) | |

Erythrovirus B19-specific Primer/Probe Set 1

(SEQ ID NO: 49)

| | |
|---|---|
| EF17 | 5′-GAAGCTCAGTTTCCTCCGAAGT-3′ |

(SEQ ID NO: 52)

| | |
|---|---|
| ER13 | 5′-ACCCCAACTAACAGTTCACGAAAC-3′ |

(SEQ ID NO: 51)

| | |
|---|---|
| EB2 | 5′-TTCTACACACCTTTGGCAGA-3′ |
| (FAM-la- | |
| beled MGB | |
| probe) | |

Erythrovirus A6/LaLi-specific Primer/Probe Set 1

(SEQ ID NO: 49)

| | |
|---|---|
| EF17 | 5′-GAAGCTCAGTTTCCTCCGAAGT-3′ |

(SEQ ID NO: 52)

| | |
|---|---|
| ER13 | 5′-ACCCCAACTAACAGTTCACGAAAC-3′ |

(SEQ ID NO: 90)

| | |
|---|---|
| EA1 | 5′-TTTACACTCCACTTGCAGAC-3′ |
| (FAM-la- | |
| beled MGB | |
| probe) | |

Erythrovirus V9/D91.1-specific Primer/Probe Set 1

(SEQ ID NO: 146)

| | |
|---|---|
| EF31 | 5′-GTCTGGATTACAAAGCTTTGTAGATTATGAGTAA-3′ |

(SEQ ID NO: 147)

| | |
|---|---|
| ER31 | 5′-GAGGGGTTCTCTAAAGGATTATCTAAAGAAAT-3′ |

(SEQ ID NO: 149)

| | |
|---|---|
| EVD | 5′-ATTTGCCCAGGACGTGTA-3′ |
| (FAM-la- | |
| beled MGB | |
| probe) | |

β-globin gene (control) primers: [

(SEQ ID NO: 167)

| | |
|---|---|
| LA1 | 5′-ACACAACTGTGTTCACTAGC-3′ |

(SEQ ID NO: 168)

| | |
|---|---|
| LA2 | 5′-CAACTTCATCCACGTTCACC-3′ |

“F” refers to the forward primer; “R” to the reverse primer. Probes are frozen at a 100 μM concentration. The working concentration of the probes is 10 μM, and are diluted 1:10 with 10 mM Tris-HCl, pH 8.0, and distributed into 100 μL aliquots. Probes can be stored at −20° C. or lower and protected from light.

Enzymes. The following enzymes are used: 2×TaqMan® Universal PCR Master Mix Applied Biosystems Cat. #4304437 or 4318157; and AmpliTaq DNA Polymerase Applied Biosystems Cat. #N801-0060 or Roche Molecular Diagnostics Cat. #1147633.

Reagents and Buffers. The following were used in the assays: QIAamp DNA Blood Mini Kit (QIAGEN Cat. No.51106); RNAse-free water; 10 mM Tris-HCl, pH 8.0; dNTPs Solution (Stratagene Cat. #200415-51) (dATP 250 μL (USB #14244), dCTP 250 μL (USB #14279), dGTP 250 μL (USB #14314), dTTP 250 μL (USB #22324), H₂O9 mL)); DNA Loading Buffer (Glycerol 50% (w/v 0.50 mL), 25 mM EDTA pH 7.4 (0.25 mL), Xylene Cyanol (5 mg), Bromophenol Blue (5 mg), Sterile H₂O (1.25 mL)); 5×TBE Buffer pH 8.3 (0.9 M Trizma Base (218.0 g), 0.9 M Boric Acid (110.0 g), 25 mM EDTA (18.8 g), dH₂O (to 4 litles)); Proteinase K (Sigma #2308) (Proteinase K (25 mg), dH₂O (1.25 mL)); Phosphate Buffered Saline; Saturated phenol; Chloroform; Isoamyl Alcohol; Solution of phenol:chloroform:isoamyl alcohol. (25:24:1); Solution of chloroform:isoamyl alcohol (24:1); Isopropanol; Absolute Alcohol; 70% alcohol; 3 M Sodium Acetate, pH 5.2; Ethidium Bromide; Buffer ATL (Qiagen Cat.#19076); Clearing Solvent (Richard Allen Scientific Cat#8301).

Equipment. Equipment used included the ABI PRISM® Sequence Detection System and a Perkin Elmer 9600 thermocycler.

Quality Control Measures. For extraction negative control, RNAse free water is used in place of clinical specimen. For an extraction positive control, amplification of β-globin DNA is used. For blood and tissue specimens, the J3-globin sequences are amplified from endogenous cellular DNA. For urine and CSF specimens, a plasmid DNA (pTOPOβ-XhoI) containing the human β-globin gene is spiked into the clinical specimen prior to DNA extraction. An assay can be interpreted as positive only if the β-globin control is amplified and erythrovirus DNA amplified products are present. An assay can be interpreted as negative only if the β-globin control is amplified and erythrovirus DNA amplified products are absent.

For RT-PCR a water control was used for each RT-PCR run as a negative control, with RNAse free water added to the master mix aliquot instead of RNA. For a positive control, a plasmid DNA (pTOPOβ-XhoI) containing the human β-globin gene was included in each RT-PCR run.

To minimize potential cross-contamination, reagent preparation, specimen processing, PCR set-up, and amplification are each carried out in physically separated laboratory areas.

Procedure for Sample Processing. QIAGEN protease stock solution was prepared by adding protease solvent (nuclease free water containing 0.04% sodium azide) to the lyophilized QIAGEN Protease provided in the QIAamp DNA Blood Kit (1.2 mL of solvent is added to the 24 mg Protease provided in the 50 column kit size; 5.5 mL of solvent is added to the 110 mg Protease provided in the 250 column kit size) and was stored at 4° C. Buffer AW1 adding the appropriate amount of absolute ethanol (200 proof) to Buffer AW1 concentrate as indicated on the bottle. For the 50 and 250 preparation kits 25 mL and 125 mL of absolute ethanol was added, respectively, and was stored at room temperature. Final buffer volumes were 44 mL and 220 mL, for the 50 and 250 mL preparation kits, respectively. Buffer AW2 was prepared by adding the appropriate amount of absolute ethanol (200 proof) to Buffer AW2 concentrate as indicated on the bottle. For the 50 and 250 preparation kits, 30 mL and 160 mL of absolute ethanol were added, respectively. Final buffer volumes were 43 mL and 226 mL, for the 50, and 250 preparation kits, respectively.

Serum, Plasma, and Amniotic Fluid Specimen Preparation

20 μL QIAGEN protease was aliquoted to 2.0 mL screw-cap microcentrifuge tubes for each specimen to be assayed plus the negative extraction control. The specimen was inverted to mix thoroughly and then, 200 μL of each sample was added to the 2.0 mL screw-cap tube containing the QIAGEN protease. After addition of specimen to the tube, 200 μL Buffer AL was added, the tube re-capped and immediately mixed by vortexing for 15 seconds. For the internal controls, 5×10⁵ copies of the linearized internal control plasmid (5 μL of pTOPOβ-XhoI at a concentration of 10⁵ copies/μL) was added to the tube containing specimen, protease and Buffer AL. The tube was re-capped and vortexed for 15 seconds. The samples were then incubated for 30 minutes at 55° C.

After the incubation, the tubes were briefly centrifuged to deposit all material in the bottom of the tube. Then, 200 μL of absolute ethanol (200 proof) to the samples, and were then mixed again by vortexing. The tubes were briefly centrifuge to deposit all material in the bottom of the tubes.

The mixtures were then applied to QIAamp spin columns that had previously been placed in a 2-mL collection tubes. The columns were then centrifuged at 6000×g (8200 rpm) for 1 minute. The QIAamp spin columns were then placed clean 2-mL collection tube, and the tubes containing the filtrate were discarded.

500 μL of Buffer AW1 was then added to each column using a separate pipet tip for each sample. The columns were then centrifuged at 6000×g (8200 rpm) for 1 minute, then placed in a clean 2-mL collection tube, and the collection tube containing the filtrate was discarded. 500 μL of Buffer AW2 was then added to each column using a separate pipet tip for each sample. The columns were then centrifuged at full speed (20,000×g or 14,000 rpm) for 3 minutes, then placed in a clean 1.5 mL microcentrifuge tube, and the collection tube containing the filtrate was discarded.

50 μL of 10 mM Tris-HCl, pH 8.0, preheated to 55° C. was then added to each column using a separate pipet tip for each sample. The columns were then incubated at room temperature for 5 minutes and then centrifuged at 6000×g (8200 rpm) for 1 minute. The columns were discarded and the microcentrifuge tubes containing the eluted DNA were stored at 4° C. (when the PCR reactions were not to be performed within 24 hours, the eluted DNA was stored at −20° C.).

Tissue Specimen Preparation

For tissue specimen preparation, the samples were first brought to room temperature and then a 25 mg piece of tissue was cut into small pieces (to decrease lysis time) and transferred into a 2 mL screw-cap microcentrifuge tube. 400 μL of Buffer ATL was then added to the tube.

20 μL of Proteinase K was then added, mixed by vortexing, and incubated at 56° C. until the tissue was completely lysed (from 1 to 3 hours), vortexing occasionally during the incubation period to disperse the sample. After the tissue was completely lysed, the tube was briefly centrifuged to deposit material in the bottom of the tube. 400 μL Buffer AL was added and the tube re-capped and immediately mixed by vortexing for 15 seconds, and then incubated for 10 minutes at 70° C.

Following incubation, 400 μL of Absolute Alcohol was added to the samples, then capped and vortexed for 15 seconds. The tubes were then briefly centrifuged to deposit all the material to the bottom of the tube. 650 μL of the mixture was then added to a QIAamp spin column without moistening the rim. The column was then placed in a new 2.0-mL collection tube and centrifuged for 1 minute at 8000 rpm. The collection tube containing the filtrate was then discarded and the spin column was then placed in a new 2.0-mL collection tube. This step was then repeated until the sample mixture from above had all been run through the spin column.

500 μL of Buffer AW1 was then added to the column. The column was then centrifuged at 6000×g (8200 rpm) for 1 minute, then placed in a clean 2-mL collection tube, and the collection tube containing the filtrate was discarded. 500 μL of Buffer AW2 was then added to the column. The column was then centrifuged at full speed (20,000×g or 14,000 rpm) for 3 minutes, then placed in a clean 1.5 ML microcentrifuge tube, and the collection tube containing the filtrate was discarded.

50 μL of 10 mM Tris-HCl, pH 8.0, preheated to 55° C. was then added to the column. The column was then incubated at room temperature for 5 minutes and then centrifuged at 6000×g (8200 rpm) for 1 minute. The column was discarded and the microcentrifuge tubes containing the eluted DNA were stored at 4° C. (when the PCR reactions were not to be performed within 24 hours, the eluted DNA was stored at −20° C.).

For Paraffin-Embedded Tissue

A small section (no more than 25 mg) of paraffin-embedded tissue was cut and placed in a 2 mL screw-cap microcentrifuge tube. 1200 μL of clearing solvent solution (Xylene free) was then added to the sample and vortexed vigorously. The tube was then centrifuge at 1500 rpm for 5 minutes at room temperature. The supernatant was then aspirated and removed without removing any of the pellet. 1200 μL of absolute alcohol was added to the pellet to remove residual solvent, and then mixed by vortexing. The tube was then centrifuged at 1500 rpm for 5 minutes at room temperature. The alcohol was then carefully removed by pipetting, without removing any of the pellet. The tube containing the pellet was then incubated at 55° C. (open cap) for 10 minutes to remove all alcohol. The pellet was then resuspend in 400 μL buffer ATL.

20 μL of Proteinase K was then added, mixed by vortexing, and incubated at 56° C. until the tissue was completely lysed (from 1 to 3 hours), vortexing occasionally during the incubation period to disperse the sample. After the tissue was completely lysed, the tube was briefly centrifuged to deposit material in the bottom of the tube. 400 μL Buffer AL was added and the tube re-capped and immediately mixed by vortexing for 15 seconds, and then incubated for 10 minutes at 70° C.

Following incubation, 400 μL of Absolute Alcohol was added to the samples, then capped and vortexed for 15 seconds. The tubes were then briefly centrifuged to deposit all the material to the bottom of the tube. 650 μL of the mixture was then added to a QIAamp spin column without moistening the rim. The column was then placed in a new 2.0-mL collection tube and centrifuged for 1 minute at 8000 rpm. The collection tube containing the filtrate was then discarded and the spin column was then placed in a new 2.0-mL collection tube. This step was then repeated until the sample mixture from above had all been run through the spin column.

500 μL of Buffer AW1 was then added to the column. The column was then centrifuged at 6000×g (8200 rpm) for 1 minute, then placed in a clean 2-mL collection tube, and the collection tube containing the filtrate was discarded. 500 μL of Buffer AW2 was then added to the column. The column was then centrifuged at full speed (20,000×g or 14,000 rpm) for 3 minutes, then placed in a clean 1.5 mL microcentrifuge tube, and the collection tube containing the filtrate was discarded.

50 μL of 10 mM Tris-HCl, pH 8.0, preheated to 55° C. was then added to the column. The column was then incubated at room temperature for 5 minutes and then centrifuged at 6000×g (8200 rpm) for 1 minute. The column was discarded and the microcentrifuge tubes containing the eluted DNA were stored at 4° C. (when the PCR reactions were not to be performed within 24 hours, the eluted DNA was stored at −20° C.).

Procedure for Real-Time PCR Reactions. The Real-time PCR reagents that were used were as follows: 2×Taqman Universal PCR Master Mix (12.5 μL); Forward Primer (22.5 μM working conc.) (1 μL); Reverse Primer (22.5 μM working conc.) (1 μL); TaqMan MGB Probe (10 μM working conc.) (0.5 μL); H₂O (5 μL); Sample DNA, positive control or negative control (5 μL); Erythrovirus standards (plasmid DNA clones at 4×10⁻² to 4×10¹² copies/μL); Positive control (plasmid DNA clone at 4×10² copies/μL); Negative control (dH₂O).

The Real-time PCR master mix was prepared by adding the following in a 1.5 mL microcentrifuge tube: RNAse-free H₂O; 2×TaqMan Universal PCR Master mix; Specific Primers (Forward primer and Reverse primer); and TaqMan Probe. 20 μL of master mix was aliquoted into each well of the 96-Well Optical Reaction Plate and then 5 μL of the appropriate DNA sample was added to the appropriate well of the 96-well Optical Reaction Plate. The plate was then covered with an Optical Adhesive Cover. The plate was then briefly centrifuged to collect the reactions at the bottom of the wells and to eliminate any air bubbles. The samples were then incubated using an ABI Prism 9700HT Sequence Detection System were as follows: 50° C. 2 minutes; 95° C. 10 minutes; then 40 cycles of 95° C., 15 sec and 60° C. 1 minute.

Detection of β-Globin Sequence. Detection of β-Globin Sequence was used as an internal assay control for all specimens. Oligonucleotides LA1 and LA2 are the primers that were used to amplify a 110 bp region of the β-globin gene. The PCR amplification procedure was performed in the Perkin Elmer 9600/9700 thermocyclers. β-globin amplicons were separated by agarose gel electrophoresis and the agarose gel was then stained with ethidium bromide and visualized under UV light.

For all the specimens, the PCR reaction were set up as follows: dNTP (0.1 M each; dATP, dTTP, dGTP, dCTP) (2 μL); 10×PCR buffer (2.5 μL); MgCl₂ (25 mM) (1.5 μL); Taq polymerase (5 U/μL) (0.125 μL); Primers LA1, LA2 (10 μM each) (1 μL each); H₂O (11.875 μL); Sample DNA, positive control or negative control (5 μL) (Positive control: pTOPOβ-XhoI at 200 copies/μL; Negative control: dH₂O). The samples were then incubated using a Perkin Elmer 9600 thermocycler using the following parameters: 94° C. 1 minutes 50 cycles of 94° C. 30 sec, 55° C., 30 sec, 72° C. 30 sec; 72° C. 10 min.

The detection of β-globin gene by PCR served as an internal assay control to indicate that DNA had been extracted and was suitable for amplification. If the β-globin gene was not amplified, DNA was extracted again (if adequate specimen is available) and the assay was repeated. If additional specimen was not available, the DNA was purified by phenol clean-up procedure and the assay was repeated.

Erythrovirus DNA Standard Curve. E. coli (DH5α strain) were transformed with plasmid (Litmus 29) containing a 5028-nt insert of erythrovirus genotype-1 (B19) DNA. For genotype 2 (A6) a corresponding plasmid had been prepared as described (Hokynar et al, 2004). After overnight growth, the plasmid DNA was isolated by the GenElute Endotoxin free Maxiptep System (Sigma), and quantified spectrophotometrically (Biorad). The copy number, as based on this data and the known construct size, was adjusted with H₂O to 1×10⁹ per μl. Erythrovirus-DNA content of the plasmid preparations was measured specifically with the Artus RealArt PCR, and a standard curve was made of 1×10⁷, 1×10⁵, 1×10³ and 1×10¹ copies of erythrovirus-DNA per μl.

### Example 1

### Simultaneous Detection and Quantitation of Erythrovirus Genotype 1 (B19), Genotype 2 (A6/Lali) and Genotype 3 (V9/D91.1) DNA by Real-time TaqMan PCR

A defined amount of erythrovirus target nucleic acid, i.e. 1.5×10⁻² to 1.5×10⁵ copies per reaction, was amplified and detected by TaqMan PCR reactions. The reaction mixture was in a final volume of 25 μL, which consisted of 12.5 μL 2×universal PCR master mix, primers (0.9 μM) of EF2 (SEQ ID NO:31) and ER2a (SEQ ID NO:33), probe of EP1a (SEQ ID NO:32) (0.2 μM), and 5 μL samples or controls. The reaction conditions included 2 min at 50° C., 10 min at 95° C. and followed by 40 cycles of 15 sec at 95° C., 1 min at 60° C. in the ABI 9700HT Sequence Detection System.

Following the real-time amplification and detection steps, quantification of the erythrovirus products was made based on standards of known target concentrations. Using the primer pairs of EF2 (SEQ ID NO:31) and ER2a (SEQ ID NO:33) and probe EP1a (SEQ ID NO:32), as few as 1.5 copy per assay reaction were detected.

In similar experiments, the assays were performed using other combinations of primers and probes derived from SEQ ID NO:12 to SEQ ID NO:166. These assays showed substantially equivalent results in simultaneously detecting all three genotypes of erythrovirus.

### Example 2

### Genotype-specific Detection, Differentiation and Quantitation among Erythrovirus Genotype 1 (B19), Genotype 2 (A6/Lali) and Genotype 3 (V9/D91.1) DNA by Real-time TaqMan PCR

A defined amount of erythrovirus target nucleic acid, i.e. 1.5×10⁻² to 1.5×10⁵ copies per reaction, was amplified and detected by TaqMan PCR reactions. The reaction mixture was in a final volume of 25 μL, which consisted of 12.5 μL 2×universal PCR master mix; genotype-specific primers (0.9 μM): SEQ ID:7 and SEQ ID:8 for genotype 1 (B119 genotype) and genotype 2 (A6 genotype) or SEQ ID NO:11 and SEQ ID NO:12 for genotype 3 (V9 genotype); genotype-specific probes (0.2 μM): SEQ ID:9 for genotype 1 (B19 genotype), SEQ ID:10 for genotype 2 (A6 genotype) or SEQ ID NO:13 for genotype 3 (V9 genotype); and 5 μL samples or controls. The reaction conditions included 2 min at 50° C., 10 min at 95° C. and followed by 40 cycles of 15 sec at 95° C., 1 min at 60° C. in the ABI 9700HT Sequence Detection System.

Following the real-time amplification and detection steps, quantification of the erythrovirus products was made based on standards of known target concentrations. Using the above genotype-specific primer pairs and corresponding probes, as few as 15 copies per assay reaction were detected. In addition, the assay provided for differentiation among genotype 1 (B19 genotype), genotype 2 (A6 genotype) and genotype 3 (V9 genotype), i.e. genotype-specific detection was confirmed.

### Example 3

### Genotype-specific Detection, Differentiation and Quantitation among Erythrovirus Genotype 1 (B19), and Genotype 2 (A6/Lali) DNA by Real-time TaqMan PCR

A first set of PCR assays (sample group 1) were performed in duplicate with ex-vivo DNA preparations known to contain erythrovirus DNA of genotypes 1 or 2, on the basis of generic (VP1) and specific (K71) qualitative nested-PCRs (Söderlund et al, 1997; Hokynar et al, 2002), and by the REALART™ Parvo B19 LIGHTCYCLER® PCR kit (Artus, Valencia Calif.) (hereinafter “REALART™”) followed by DNA melting point analysis, as described (Hokynar et al, 2004). This sample group included 7 biopsies from skin and 2 from tonsils. The DNA from the skin samples was isolated by phenol-chloroform extraction followed by ethanol-sodium acetate precipitation. The DNA from the tonsil samples was isolated using the QIAamp DNA Mini kit (Qiagen) according to the manufacturer's instructions (Hokynar et al, 2002; Kakkola et al, 2004).

The second set of PCR assayss (sample group 2) were also performed. The second group included 5 biopsies from skin, 2 biopsies from tonsils, 2 biopsies from synovia; and 1 sample of serum. The DNA from all the samples was isolated using the QIAamp DNA Mini kit. The PCR assays were performed in duplicate (with each primer-probe pair) with the DNA preparations in dilutions 10⁰, 10¹ and, 10². The duplicate tests with undiluted DNA were repeated.

First, a control was peformed using serial dilutions of target DNA of erythrovirus genotypes 1 and 2. Both of the primer-probe pairs were tested by using as standard three serial dilutions of the genotype-1 plasmid, and as template two dilutions of the genotype-2 plasmid. The negative control was water, and the background control was Litmus 29 vector (1×10⁶ copies per μl) devoid of insert (genotypes 1 or 2). Each primer-probe pair was able to amplify and detect without difficulty both of the erythrovirus plasmids. The negative control did not prduce a signal. The background control yielded a negligible, barely detectable signal (4.1×10⁻¹ copies per μl).

The results are provided in Tables 1 and 2. In the first set of experiments with undiluted DNA of sample group 1 (tested in duplicate), the primer-probe pair EF2 (SEQ ID NO:31), ER2a(SEQ ID NO:33), EP1a (SEQ ID NO:32) gave positive results for 3/5 skin samples (DNA isolated by phenol-chloroform extraction) and for 2/2 tonsillar samples (DNA isolated using Qiagen system) (Table 1). The primer-probe pair ER4 (SEQ ID NO:44), EF6 (SEQ ID NO:38), EP5 (SEQ ID NO:41) gave positive results for 2/5 skin samples and 2/2 tonsillar samples (Table 1).

Additional tissues were obtained; their DNA was isolated by QIAamp and studied (in duplicate) in serial dilutions. The results of the second set of experiments are provided in Table 2. The results show that with undiluted DNA (tested repeatedly), the two primer-probe pairs gave positive results for 2/3 samples of genotype 1 and 3/5 samples of genotype 2 (Table 2). Moreover, with the DNA diluted 1 in 10, both of the primer-probe pairs gave positive results for all the 8 samples (3 of genotype 1, and 5 of genotype 2). With the two negative control samples, both primer-probe pair gave negative results (Table 2).

TABLE 1
Sample group I.

| | | | | | | | | Result copies/μl | | Result copies/μl | |
|---|---|---|---|---|---|---|---|---|---|---|---|
| Sample | | | | | VP1- | K71- | Results | (EF2, ER2a, EP1a) | | (ER4, EF6, EP5) | |
| code | Tissue | Dilution | Genotype | DNA isolation | PCR | PCR | REALART ™ | sample | replicate | sample | replicate |
| FaAh | tonsil | — | 1 | QIAmp | + | − | 2.1 × 10³ IU/μl | 3.6 × 10⁴ | 4.4 × 10⁴ | 2.7 × 10⁴ | 2.2 × 10⁴ |
| | | | | (Qiagen) | | | | | | | |
| ViAn | tonsil | — | 2 | QIAmp | | | 1.4 × 10¹ IU/μl | 3.8 × 10¹ | 2.1 × 10¹ | 3.3 × 10¹ | 3.4 × 10¹ |
| | | | | (Qiagen) | | | | | | | |
| HeKl | skin | 1:10 | 1 | phenol- | + | − | 1.1 × 10¹ IU/μl | 1.6 × 10¹ | 1.2 × 10¹ | 1.9 × 10¹ | 4.0 × 10¹ |
| | biopsy | | | chloroform | | | | | | | |
| KuTu | skin | 1:10 | 2 | phenol- | + | + | N/A | 5.0 × 10⁻¹ | 4.8 × 10⁻¹ | — | — |
| | biopsy | | | chloroform | | | | | | | |
| CaSa | skin | 1:10 | 2 | phenol- | + | + | 2.8 × 10⁰ IU/μl | 1.1 × 10⁰ | 2.5 × 10⁻¹ | 8.7 × 10⁻¹ | 1.5 × 10⁰ |
| | biopsy | | | chloroform | | | | | | | |
| SaJa | skin | 1:10 | 2 | phenol- | + | + | N/A | — | — | — | — |
| | biopsy | | | chloroform | | | | | | | |
| NuAn | skin | 1:10 | 2 | phenol- | + | + | N/A | — | — | — | — |
| | biopsy | | | chloroform | | | | | | | |
| PuPe | skin | 1:10 | negat. | phenol- | − | − | N/A | — | — | — | — |
| | biopsy | | | chloroform | | | | | | | |
| KaEe | skin | 1:10 | negat. | phenol- | − | − | N/A | — | — | — | — |
| | biopsy | | | chloroform | | | | | | | |

* An “N/A” indicates the test was not performed. A “−” indicates a negative result.

TABLE 2

| | | | | | | | Result copies/μl | | Result copies/μl | |
|---|---|---|---|---|---|---|---|---|---|---|
| | DNA | | | VP1- | K71- | Results | (EF2, ER2a, EP1a) | | (ER4, EF6, EP5) | |
| Sample code | isolation | genotype | dilution | PCR | PCR | REALART ™ | sample | replicate | sample | replicate |
| AdMi | Qiagen | 1 | DNA sample | + | − | 3.458 × 10² IU/μl | I — | I — | I — | I — |
| | | | | | | | II — | II — | II — | II — |
| (skin biopsy) | | | 1:10 | | − | | 6.8 × 10¹ | 7.8 × 10¹ | 6.9 × 10¹ | 6.6 × 10¹ |
| | | | 1:100 | | | | 7.2 × 10⁰ | 1.5 × 10¹ | 1.8 × 10¹ | 1.8 × 10¹ |
| FaAh | Qiagen | 1 | DNA sample | + | − | 2.1 × 10³ IU/μl | I 1.0 × 10⁴ | I 1.1 × 10⁴ | I 8.3 × 10³ | I 8.5 × 10³ |
| | | | | | | | II 1.2 × 10⁴ | II 1.2 × 10⁴ | II 6.6 × 10³ | II 7.5 × 10³ |
| (tonsil) | | | 1:10 | | − | | 3.0 × 10³ | 2.9 × 10³ | 3.0 × 10³ | 2.8 × 10³ |
| | | | 1:100 | | | | 3.6 × 10² | 3.9 × 10² | 4.1 × 10² | 4.0 × 10² |
| LaMa | Qiagen | 1 | DNA sample | + | − | 3.853 × 10² IU/μl | I — | I — | I — | I — |
| | | | | | | | II — | II — | II — | II — |
| (skin biopsy) | | | 1:10 | | − | | 2.7 × 10⁰ | 3.9 × 10⁰ | 5.3 × 10⁰ | 7.0 × 10⁰ |
| | | | 1:100 | | | | — | 8.7 × 10⁻¹ | 2.4 × 10⁰ | 3.0 × 10⁰ |
| LiKi | Qiagen | 2 | DNA sample | + | + | 1.649 × 10¹ IU/μl | I — | I — | I — | I — |
| | | | | | | | II — | II — | II — | II — |
| (skin biopsy) | | | 1:10 | + | + | | 1.3 × 10¹ | 1.5 × 10¹ | 7.9 × 10⁰ | 5.0 × 10⁰ |
| | | | 1:100 | | | | 1.4 × 10⁰ | — | 1.9 × 10⁰ | — |
| LoPä | Qiagen | 2 | DNA sample | + | + | 2.250 × 10¹ IU/μl | I 7.4 × 10² | I 2.5 × 10¹ | I 2.5 × 10¹ | I 2.3 × 10¹ |
| | | | | | | | II 5.6 × 10⁰ | II 5.0 × 10⁰ | II 6.1 × 10⁰ | II 9.7 × 10⁰ |
| (skin biopsy) | | | 1:10 | + | + | | 1.1 × 10¹ | 1.1 × 10¹ | 1.3 × 10¹ | 1.6 × 10¹ |
| | | | 1:100 | | | | 4.1 × 10⁰ | 1.8 × 10⁰ | 2.4 × 10⁰ | 2.2 × 10⁰ |
| RiAl | Qiagen | 2 | DNA sample | + | + | 6.140 × 10¹ IU/μl | I 8.6 × 10⁰ | I 9.1 × 10⁰ | I 9.1 × 10⁰ | I 4.6 × 10⁰ |
| | | | | | | | II 9.8 × 10⁻¹ | II 1.5 × 10⁰ | II 7.5 × 10⁻¹ | II 5.7 × 10⁻¹ |
| (skin biopsy) | | | 1:10 | + | + | | 4.5 × 10¹ | 4.9 × 10¹ | 5.5 × 10¹ | 5.2 × 10¹ |
| | | | 1:100 | | | | 5.4 × 10⁰ | 4.8 × 10⁰ | 7.1 × 10⁰ | 1.2 × 10¹ |
| TiCa | Qiagen | 2 | DNA sample | + | − | — | I — | I — | I — | I — |
| (skin biopsy) | | | 1:10 | + | + | 9.418 × 10⁰ IU/μl | 7.8 × 10⁰ | 9.4 × 10⁰ | 7.4 × 10⁰ | 1.1 × 10¹ |
| | | | 1:100 | | | | 2.6 × 10⁰ | 1.4 × 10⁰ | 4.3 × 10⁰ | 2.2 × 10⁰ |
| ViAn | Qiagen | 2 | DNA sample | | | 1.449 × 10¹ IU/μl | I 6.5 × 10¹ | I 6.6 × 10¹ | I 6.3 × 10¹ | I 6.3 × 10¹ |
| | | | | | | | II 1.3 × 10¹ | II 1.7 × 10¹ | II 2.3 × 10¹ | II 2.4 × 10¹ |
| (tonsil) | | | 1:10 | | | | 2.0 × 10¹ | 1.5 × 10¹ | 1.7 × 10¹ | 1.5 × 10¹ |
| | | | 1:100 | | | | 4.9 × 10⁰ | 3.6 × 10⁰ | — | — |
| LaKa (scrum) | Qiagen | Negat. | DNA sample | | | | — | — | — | — |
| SuMe (synovium) | Qiagen | Negat. | DNA sample | | | | — | — | — | — |

* The roman numerals (I, II) indicate that the test was run once (I) or twice (I, II). A “−” indicates a negative result.

The quantitative results of the positive samples were within 1 log of the reference for 7/8 tissue samples and within 2 logs for the remaining 1/8 tiussue sample. Both of the two primer-probe pairs amplified and detected in real-time PCR, both cloned and native (tissue-derived) erythrovirus DNA of either genotype 1 or genotype 2 in all the positive samples studied.

Of note, many of the tissue-derived, low-copy-number DNA preparations yielded negative PCR results when studied undiluted. This phenomenon was seen with either DNA isolation method, phenol-chloroform or QIAamp (Qiagen). A simple one-log dilution (1 in 10) of the DNA preparation was sufficient to abrogate the block, strongly suggesting that its mechanism is DNA polymerase inhibition due to in vitro (phenol, with the former method) or ex-vivo inhibitors (with either method).

### Example 4

### Detection of Erythrovirus Genotype 3 (V9/D91.1) DNA by Real-time TaqMan PCR

The study comprised two variants of genotype 3, the V9 and the D91.1, in plasmid form and serum. The genome of isolate V9 (V9-C22 Gen Bank accession number AJ249437) was inserted into (Hokynar et al. 2004) plasmid pcDNA2.1 (Invitrogen Life Technologies, Paisley, United Kingdom); and the genome of isolate D91.1 was inserted into plasmid pcDNA3.1HisB (Invitrogen).

Plasmid DNA was isolated by GenElute Endotoxin free Maxi Prep kit (Sigma, St. Louis, Mo.) from overnight-grown E. coli (strain DH5α). Dilution series of plasmids (10⁹ to 10⁰ copies per μl) were prepared. The erythrovirus DNA concentrations were measured by the Artus Real Art Parvo B19 PCR (Hokynar et al. 2004), and dilutions 10⁴ to 10⁰ were selected for use.

Serum samples including either genotype were also acquired (V9-E00.2 and D91.1). DNA was prepared by phenol-chloroform extraction followed by ethanol-NaAc precipitation, and dilutions of 10⁴ to 10² were used. Serum D91.1 contained ≧1×10⁴ copies per μl of genotype-3 DNA. The concentration could not be measured accurately by the Artus kit, due to its low detection sensitivity for isolate D91.1 (˜3 log lower than for V9, or genotypes 1 or 2) (Hokynar et al. 2004). Serum V9 was verified with a qualitative VP1-PCR(Söderlund et al. 1997; primers 5 and 6). One erythrovirus DNA negative serum (LaKa) and PCR-grade water were used as negative controls in each PCR run.

TABLE 3

| | | | | Result copies/μl | | Result copies/μl | | |
|---|---|---|---|---|---|---|---|---|
| | SAMPLE | Concentration | | (EF2, ER2a, EP1a) | | (ER4, EF6, EP5) | | Results |
| Sample name | MATERIAL | or dilution | DNA isolation | sample | replicate | sample | replicate | REALART ™ |
| pV9 10⁴ | plasmid-DNA | 10⁴ copies/μl | maxiprep kit (Sigma) | 3.7 × 10⁴ | 3.1 × 10⁴ | 3.5 × 10⁴ | 3.0 × 10⁴ | 5.819 × 10³ |
| pV9 10³ | plasmid-DNA | 10³ copies/μl | maxiprep kit (Sigma) | 1.4 × 10⁴ | 5.2 × 10³ | 1.3 × 10⁴ | 3.7 × 10³ | 5.922 × 10² |
| pV9 10² | plasmid-DNA | 10² copies/μl | maxiprep kit (Sigma) | 1.0 × 10³ | 1.1 × 10³ | 6.7 × 10² | 7.6 × 10² | 5.958 × 10¹ |
| pV9 10¹ | plasmid-DNA | 10¹ copies/μl | maxiprep kit (Sigma) | 1.1 × 10² | 1.5 × 10² | 5.7 × 10¹ | 9.2 × 10¹ | 8.6 × 10⁰ |
| pV9 10⁰ | plasmid-DNA | 10⁰ copies/μl | maxiprep kit (Sigma) | 3.9 × 10¹ | 4.6 × 10¹ | 1.9 × 10¹ | 1.8 × 10¹ | 5.772 × 10⁰ |
| pD91.1 10⁴ | plasmid-DNA | 10⁴ copies/μl | maxiprep kit (Sigma) | 2.8 × 10⁴ | 3.0 × 10⁴ | 2.7 × 10⁴ | 2.7 × 10⁴ | 1.279 × 10¹ |
| pD91.1 10³ | plasmid-DNA | 10³ copies/μl | maxiprep kit (Sigma) | 9.6 × 10³ | 8.2 × 10³ | 6.9 × 10³ | 8.4 × 10³ | 1.599 × 10⁰ |
| pD91.1 10² | plasmid-DNA | 10² copies/μl | maxiprep kit (Sigma) | 6.6 × 10² | 8.8 × 10² | 6.0 × 10² | 4.6 × 10² | 3.927 × 10⁰ |
| pD91.1 10¹ | plasmid-DNA | 10¹ copies/μl | maxiprep kit (Sigma) | 8.9 × 10¹ | 1.1 × 10² | 6.6 × 10¹ | 4.4 × 10¹ | negat. |
| pD91.1 10⁰ | plasmid-DNA | 10⁰ copies/μl | maxiprep kit (Sigma) | 8.4 × 10¹ | 4.7 × 10¹ | 2.5 × 10¹ | 3.7 × 10¹ | negat. |
| V9 1:100 | serum | 1:100 | phenol-chloroform | 1.5 × 10² | 1.4 × 10² | 8.8 × 10¹ | 6.6 × 10¹ | negat. |
| V9 1:1000 | serum | 1:1000 | phenol-chloroform | 2.8 × 10¹ | 4.4 × 10¹ | 1.4 × 10¹ | 2.3 × 10¹ | negat. |
| V9 1:10000 | serum | 1:10000 | phenol-chloroform | negat. | 6.5 × 10⁰ | negat | 2.7 × 10⁰ | negat. |
| V9 1:100000 | serum | 1:100000 | phenol-chloroform | negat. | negat. | negat. | negat. | negat. |
| D91.1 1:100 | serum | 1:100 | phenol-chloroform | 6.4 × 10⁵ | 6.9 × 10⁵ | 8.8 × 10⁵ | 9.2 × 10⁵ | 8.297 × 10¹ |
| D91.1 1:1000 | serum | 1:1000 | phenol-chloroform | 7.0 × 10⁴ | 5.7 × 10⁴ | 7.9 × 10⁴ | 6.0 × 10⁴ | 6.985 × 10⁰ |
| D91.1 1:10000 | serum | 1:10000 | phenol-chloroform | 8.1 × 10³ | 8.5 × 0³ | 6.9 × 10³ | 7.6 × 10³ | negat. |
| D91.1 1:100000 | serum | 1:100000 | phenol-chloroform | 7.1 × 10² | 1.0 × 10³ | 4.4 × 10² | 6.1 × 10² | negat. |
| LaKa | serum | — | Qiagen | negat. | negat. | negat. | negat. | negat. |
| (negat. control) | | | | | | | | |
| H₂O | H₂O | — | — | negat | negat. | negat. | negat. | negat. |

The results are shown in Table 3. Both of the primer-probe sets EF2 (SEQ ID NO:31), ER2a (SEQ ID NO:33), EP1a (SEQ ID NO:32) and ER4 (SEQ ID NO:44), EF6 (SEQ ID NO:38); EP5 (SEQ ID NO:41) detected and amplified reproducibly both plasmids and serum samples of erythrovirus genotype 3. All the negative controls gave negative results. Detection sensitivity with the Focus method appeared higher than with the reference method. The difference with isolate V9 was ˜1-2 logs; and with isolate D91.1, ˜3-4 logs or more (Table 1).

It is evident from the above results and discussion that the subject invention provides an important new means for the detection of human erythrovirus as well as differentiating between different human erythrovirus genotypes. As such, the subject methods and systems find use in a variety of different applications, including research, medical, therapeutic, military and other applications. Accordingly, the present invention represents a significant contribution to the art.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of detecting human erythrovirus (EV) nucleic acid in a sample, the method comprising:
detecting the presence or absence of a nucleic acid in a sample suspected of having human EV nucleic acid, the nucleic acid having at least 20 consecutive nucleotides of a target sequence of
AAATTTTNGCGGGCTTTTTNCCCGCCTTATGCAAAT
or a complement thereof, where N is a nucleotide chosen from a nucleotide at a corresponding position in at least one of a B19 genome, A6 genome, or V9 genome as shown in FIG. 1;
wherein detection of the presence of the nucleic acid having the target sequence indicates the sample contains human EV nucleic acid.

2. The method of claim 1, wherein said detecting is by nucleic acid-based amplification.

3. The method of claim 2, wherein the amplification product is about 30 nucleotides or less in length.

4. A method of detecting human erythrovirus (EV) nucleic acid in a sample, the method comprising:
detecting the presence or absence of a nucleic acid in a sample suspected of having human EV nucleic acid, the nucleic acid having at least 20 consecutive nucleotides of a target sequence of
TAACAGGTATTTATACTANNTNTTAANATACTAACATGGAGCTATTTNGNGGNGTNNT
NCANNTTTCNTCTAANNTTNTNGACTGTGCTAANGATAACTGGTGGTGCTCTNTNCTN
GANTTAGANACTTCTGACTGGGAACCACTAACNCANNCTAACAGANTAATGGCAATA
TANTTAAGNANTGTNGCTTCTAANCTNGANTTTACNGGGGGGCCNCTNGCNGGNTGC
TTNTACTTTTTTCANGTNGAATGTAACAAATTTGANGAAGGCTA
or a complement thereof, where N is a nucleotide chosen from a nucleotide a corresponding position in at least one of a B19 genome, A6 genome, or V9 genome as shown in FIG. 1;
wherein detection of the presence of the nucleic acid having the target sequence indicates the sample contains human EV nucleic acid.

5. The method of claim 4, wherein said detecting is by nucleic acid-based amplification.

6. The method of claim 5, wherein the amplification product is about 274 nucleotides or less in length.

7. A method of detecting human erythrovirus (EV) nucleic acid in a sample, the method comprising:
detecting the presence or absence of a nucleic acid in a sample suspected of having human EV nucleic acid, the nucleic acid having at least 20 consecutive nucleotides of a target sequence of
AAATTTTTGCCAGGNATGACTACNAAAGGNAANTANTTTAGAGATGGAGAGCAGTTT
ATAGAAAANTANNTAATGAAAAAAATNCCTTTAAATGTNGTATGGTGTGTNACNAAT
ATTGANGGNTATATAGANACCTGTATTTC
or a complement thereof, where N is a nucleotide chosen from a nucleotide a corresponding position in at least one of a B19 genome, A6 genome, or V9 genome as shown in FIG. 1;
wherein detection of the presence of the nucleic acid having the target sequence indicates the sample contains human EV nucleic acid.

8. The method of claim 7, wherein said detecting is by nucleic acid-based amplification.

9. The method of claim 8, wherein the amplification product is about 316 nucleotides or less in length.

10. A method of detecting human erythrovirus (EV) nucleic acid in a sample, the method comprising:
detecting the presence or absence of a nucleic acid in a sample suspected of having human EV nucleic acid, the nucleic acid having at least 20 consecutive nucleotides of a target sequence of
TAAAGTTTCAAACNATGGTAAANTGGNTNTGTGAAAACAGAGTNTTTACNGAN
GANAANTGGAANNTAGTNGANTTTAANCANTANACNTTANTAAGNAGNAGTCANAG
TGGNAGNTTTCAAATNCAAAGTGCNNTAAANNTAGCNATTTATAAAGCNACTAANTT
AGTNCCNACNAGNACATTNNTNNTNCATNCAGACTTTGAGCAGGTTANNTGNATTAA
AGANAATAAAATNGTNAAANTNTTANTNTGNCANAANTATGANCCNCTNTTAGTGGG
NCANCATGTNTTAANGTGGATTGANAAAAAATGTGG
or a complement thereof, where N is a nucleotide chosen from a nucleotide a corresponding position in at least one of a B19 genome, A6 genome, or V9 genome as shown in FIG. 1;
wherein detection of the presence of the nucleic acid having the target sequence indicates the sample contains human EV nucleic acid.

11. The method of claim 10, wherein said detecting is by nucleic acid-based amplification.

12. The method of claim 11, wherein the amplification product is about 316 nucleotides or less in length.

13. A method of detecting human erythrovirus (EV) nucleic acid in a sample, the method comprising:
detecting the presence or absence of a nucleic acid in a sample suspected of having human EV nucleic acid, the nucleic acid having at least 20 consecutive nucleotides of a target sequence of
CCAAGTACNGGAAAAACAAANTTGGCAATGGCNATTGCTAAAANTGTNCCAGTNTAT
GGNATGGTNAANTGGAATAATGAAAANTTTCCATTTAATGATGTAGCNGGGAAAAGN
TTGGTGGTCTGGGATGAAGGNATTATTAAGTCNACNATTGTNGAAGCTGCAAAAGCC
ATTTTAGGNGGNCANee CCNACCAGGGTAGATCANAAAATGCGTGGNAGTGTNGCNGTG
CCNGGNGTN+E CCNGTGGNNATAACCAGCAATGGTGACATTACNTTTGTTGTNAGNGGN
AANACNACNACAACTGTNCATGCTAAAGCCTTAAANGANCGNATGGTAAAGNTAAA
CTTTACNNTAAGATGNAGCCCTGACATGGGNTTACTNACAGAGGCTGATGTACANCA
NTGGCTNACNTGGTGTAATGCACAAAGCTGGNNCCACTATGAAAACTGGGCAATAAA
CTACACNTTTGATTTCCCTGGAATNAATGCAGATGCCCTCCACCCAGACCTNCAAACC
NNCCCNATTGTCNCAGACACCAGTNTCAGCAGCAGTGGTGGTGAAAGCTCTGAAGAA
CTCNGTGAAAGCAGCTTTTTNAACCTCATCACNCCAGGCGCCTGGAACANTGAAACC
CCGCGCTCTAGTACNCCCNTCCCCGGGACCAGTTCAGGAGAATCATTTGTCGGAAGC
NCAGTTTCCTCCGAAGTNGTAGCNGCNTCGTGGGANGAAGCNTTNTACACNCCNNTN
GCNGANCAGTTTCGTGAACTGTTAGTNGGGGTTGA
or a complement thereof, where N is a nucleotide chosen from a nucleotide a corresponding position in at least one of a B19 genome, A6 genome, or V9 genome as shown in FIG. 1;
wherein detection of the presence of the nucleic acid having the target sequence indicates the sample contains human EV nucleic acid.

14. The method of claim 13, wherein said detecting is by nucleic acid-based amplification.

15. The method of claim 14, wherein the amplification product is about 776 nucleotides or less in length.

16. A method of detecting human erythrovirus (EV) nucleic acid in a sample, the method comprising:
detecting the presence or absence of a nucleic acid in a sample suspected of having human EV nucleic acid, the nucleic acid having at least 20 consecutive nucleotides of a target sequence of
CAGTTTCGTGAACTGTTAGTNGGGGTTGANTATGTNTGGGANGGTGTNAGGGGNTTN
CCTGTNTGNTGTGTGNANCATATNAANAANAGTGGGGGAGGNTTNGGNCTTTGNCCN
CATTGNATTAATGTNGGNGCTTGGTATAATGGATGGAANTTTNGNGANTTTACNCCA
GANTTNGTNCGNTGNAGNTGCCATGTNGGAGCNTCTAANCCNTTTTCTGTGCTAACNT
GNAAAAAATGTGCTTACNTGTCTGGATT
or a complement thereof, where N is a nucleotide chosen from a nucleotide a corresponding position in at least one of a B19 genome, A6 genome, or V9 genome as shown in FIG. 1;
wherein detection of the presence of the nucleic acid having the target sequence indicates the sample contains human EV nucleic acid.

17. The method of claim 16, wherein said detecting is by nucleic acid-based amplification.

18. The method of claim 17, wherein the amplification product is about 257 nucleotides or less in length.

19. A method of detecting human erythrovirus (EV) nucleic acid in a sample, the method comprising:
detecting the presence or absence of a nucleic acid in a sample suspected of having human EV nucleic acid, the nucleic acid having at least 20 consecutive nucleotides of a target sequence of
AAAAAATGTGCTTACNTGTCTGGATTNCAAAGNTTTGTAGATTATGAGTAAANNNAN
TNNCAANTGGTGGGAAAGNNNTGANAAATTTGCNNAAGNNGTGTATNAGCANTTTGT
NNAATTTTATGAAAANGNTACTGNNACAGACTTAGAGCTTATTCAAATNTTAAAAGA
NCANTANAANATTTCTTTAGATAATCCNNTAGAAAACCCNTCNTCNNTNTTTGACTTA
GTTGCTCGNATTAAAANTAANCTTAAANACNCTCCAGACNTATATAGTCATCATTTTC
ANAGNCATGGACAGTTATNTGACCACCCCCATGCCTTATCANCCAGTANCAGTNNTN
CAGAACCTAGAGGAGAANATGCAGTATTATCTAGTGAAGACTTACACAAGCCTGGGC
AAGTTAGCNTACAANTACCCGGTACTAACTATNTTGGNCCTGGCAATGAGCTACAAG
CTGGGCCNCCGCANANTGCTGTNGACAGTGCTGCAAGGATTCATGACTTTAGGTATA
GCCAANTGGCTAAGNTGGGAATAAATCCNTATACNCATTGGACNGTAGCAGATGANG
ANNTNTTAAAAAATATAAAAAATGAAACNGGGTTTCAAGCACAAGNAGTAAAAGAN
TACTTTACTTTAAAAGGTGCAGCTGCCCCTGTGGCCCATTTTCAAGGAAGTTTNCCGG
AAGTNCCCGCNTACAACGCCTCAGAAAANTACCCNAGCATGACTTCAGTTAANTCTG
CAGAAGCC
or a complement thereof, where N is a nucleotide chosen from a nucleotide a corresponding position in at least one of a B19 genome, A6 genome, or V9 genome as shown in FIG. 1;
wherein detection of the presence of the nucleic acid having the target sequence indicates the sample contains human EV nucleic acid.

20. The method of claim 19, wherein said detecting is by nucleic acid-based amplification.

21. The method of claim 20, wherein the amplification product is about 751 nucleotides or less in length.

22. A method of detecting human erythrovirus (EV) nucleic acid in a sample, the method comprising:
detecting the presence or absence of a nucleic acid in a sample suspected of having human EV nucleic acid, the nucleic acid having at least 20 consecutive nucleotides of a target sequence of
TNCCACANAGTGGNCCAATTGGNGGTATTAAATCNATGGGAATNACTACCNTAGTTC
AGTATGCNGTGGGAATTATGACAGTNACTANGACNTTTAAATTGGGNCCNCGNAANG
CTACNGGNNGGTGGAATCCNCANCCTGGNGTNTATCCNCCNCANGCAGCNGGTCATT
TACCATATGTACTNTATGACCCNACAGCTACAGATGCAAANCAACACCACAGNCANG
GATANGAAAAGCCTGAAGAATTGTGGACNGCCAAAAGCCGTGTGCACCCATTGTAAA
CANTCCCCACCGTGNCCTCAGCCAGGANNCGTNACNNAb NCGNCCNCCNGTNCCNCCC
AGANTNTANNTGCCCCCNCCNNTACCNNNNAGNNANCCNNNNNNAAAAGATANANA
NNNTGTAGAATNTAANTNNNTAACNNGNTATGAACAACATGTNATTAGAATG
or a complement thereof, where N is a nucleotide chosen from a nucleotide a corresponding position in at least one of a B19 genome, A6 genome, or V9 genome as shown in FIG. 1;
wherein detection of the presence of the nucleic acid having the target sequence indicates the sample contains human EV nucleic acid.

23. The method of claim 22, wherein said detecting is by nucleic acid-based amplification.

24. The method of claim 23, wherein the amplification product is about 450 nucleotides or less in length.

25. A kit comprising a primer pair suitable for production of an amplification product of a nucleotide sequence of one or more of Target Regions I, II, III, IV, V, VI, VII, or VIII.

26. The kit of claim 25 further comprising a probe for detection of the amplification product.

27. The kit of claim 25, wherein the kit comprises at least two primers pairs for production of amplification products of at least two of the Target Regions.

28. An isolated nucleic acid of at least 20 contiguous nucleotides of:
(i) AAATTTTNGCGGGCTTTTTNCCCGCCTTATGCAAAT, or a complement thereof, wherein the nucleic acid is not more than about 30 nucleotides in length;
(ii)TAACAGGTATTTATACTANNTNTTAANATACTAACATGGAGCTATTTNGNG GNGTNNTNCANNTTTCNNTCTAANNTTNTNGACTGTGCTAANGATAACTGGTGGTGCTC TNTNCTNGANTTAGANACTTCTGACTGGGAACCACTAACNCANNCTAACAGANTAAT GGCAATATANTTAAGNANTGTNGCTTCTAANCTNGANTTTACNGGGGGGCCNCTNGC NGGNTGCTTNTACTTTTTTCANGTNGAATGTAACAAATTTGANGAAGGCTA, or a complement thereof, wherein the nucleic acid is not more than about 274 nucleotides in length;
(iii)AAATTTTTGCCAGGNATGACTACNAAAGGNAANTANTTTAGAGATGGAGA GCAGTTTATAGAAAANTANNTAATGAAAAAAATNCCTTTAAATGTNGTATGGTGTGT NACNAATATTGANGGNTATATAGANACCTGTATTTC, or a complement thereof, wherein the nucleic acid is not more than about 316 nucleotides in length;
(iv)TAAAGTTTCAAACNATGGTAAANTGGNTNTGTGAAAACAGAGTNTTTACNG ANGANAANTGGAANNTAGTNGANTTTAANCANTANACNTTANTAAGNAGNAGTCANAGTGGNAGNTTTCAAATNCAAAGTGCNNTAAANNTAGCNATTTATAAAGCNACTAANTTAGTNCCNACNAGNACATTNNTNNTNCATNCAGACTTTGAGCAGGTTANNTGNATT AAAGANAATAAAATNGTNAAANTNTTANTNTGNCANAANTATGANCCNCTNTTAGTG GGNCANCATGTNTTAANGTGGATTGANAAAAAATGTGG, or a complement thereof, wherein the nucleic acid is not more than about 316 nucleotides in length;
(v)CCAAGTACNGGAAAAACAAANTTGGCAATGGCNATTGCTAAAANTGTNCCA GTNTATGGNATGGTNAANTGGAATAATGAAAANTTTCCATTTAATGATGTAGCNGGG AAAAGNTTGGTGGTCTGGGATGAAGGNATTATTAAGTCNACNATTGTNGAAGCTGCA AAAGCCATTTTAGGNGGNCANCCNACCAGGGTAGATCANAAAATGCGTGGNAGTGTNGCNGTGCCNGGNGTNCCNGTGGNNATAACCAGCAATGGTGACATTACNTTTGTTGTNAGNGGNAANACNACNACAACTGTNCATGCTAAAGCCTTAAANGANCGNATGGTAAA GNTAAACTTTACNNTAAGATGNAGCCCTGACATGGGNTTACTNACAGAGGCTGATGT ACANCANTGGCTNACNTGGTGTAATGCACAAAGCTGGNNCCACTATGAAAACTGGGC AATAAACTACACNTTTGATTTCCCTGGAATNAATGCAGATGCCCTCCACCCAGACCTNCAAACCNNCCCNATTGTCNCAGACACCAGTNTCAGCAGCAGTGGTGGTGAAAGCTCT GAAGAACTCNGTGAAAGCAGCTTTTTNAACCTCATCACNCCAGGCGCCTGGAACANT GAAACCCCGCGCTCTAGTACNCCCNTCCCCGGGACCAGTTCAGGAGAATCATTTGTCG GAAGCNCAGTTTCCTCCGAAGTNGTAGCNGCNTCGTGGGANGAAGCNTTNTACACNC CNNTNGCNGANCAGTTTCGTGAACTGTTAGTNGGGGTTGA, or a complement thereof, wherein the nucleic acid is not more than about 776 nucleotides in length;
(vi)CAGTTTCGTGAACTGTTAGTNGGGGTTGANTATGTNTGGGANGGTGTNAGG GGNTTNCCTGTNTGNTGTGTGNANCATATNAANAANAGTGGGGGAGGNTTNGGNCTT TGNCCNCATTGNATTAATGTNGGNGCTTGGTATAATGGATGGAANTTTNGNGANTTTA CNCCAGANTTNGTNCGNTGNAGNTGCCATGTNGGAGCNTCTAANCCNTTTTCTGTGCT AACNTGNAAAAAATGTGCTTACNTGTCTGGATT, or a complement thereof, wherein the nucleic acid is not more than about 257 nucleotides in length;
(vii)AAAAAATGTGCTTACNTGTCTGGATTNCAAAGNTTTGTAGATTATGAGTAA ANNNANTNNCAANTGGTGGGAAAGNNNTGANAAATTTGCNNAAGNNGTGTATNAGC ANTTTGTNNAATTTTATGAAAANGNTACTGNNACAGACTTAGAGCTTATTCAAATNTT AAAAGANCANTANAANATTTCTTTAGATAATCCNNTAGAAAACCCNTCNTCNNTNTT TGACTTAGTTGCTCGNATTAAAANTAANCTTAAANACNCTCCAGACNTATATAGTCAT CATTTTCANAGNCATGGACAGTTATNTGACCACCCCCATGCCTTATCANCCAGTANCA GTNNTNCAGAACCTAGAGGAGAANATGCAGTATTATCTAGTGAAGACTTACACAAGC CTGGGCAAGTTAGCNTACAANTACCCGGTACTAACTATNTTGGNCCTGGCAATGAGC TACAAGCTGGGCCNCCGCANANTGCTGTNGACAGTGCTGCAAGGATTCATGACTTTA GGTATAGCCAANTGGCTAAGNTGGGAATAAATCCNTATACNCATTGGACNGTAGCAG ATGANGANNTNTTAAAAAATATAAAAAATGAAACNGGGTTTCAAGCACAAGNAGTA AAAGANTACTTTACTTTAAAAGGTGCAGCTGCCCCTGTGGCCCATTTTCAAGGAAGTT TNCCGGAAGTNCCCGCNTACAACGCCTCAGAAAANTACCCNAGCATGACTTCAGTTA ANTCTGCAGAAGCC, or a complement thereof, wherein the nucleic acid is not more than about 751 nucleotides in length; or
(viii)TNCCACANAGTGGNCCAATTGGNGGTATTAAATCNATGGGAATNACTACC NTAGTTCAGTATGCNGTGGGAATTATGACAGTNACTANGACNTTTAAATTGGGNCCNCGNAANGCTACNGGNNGGTGGAATCCNCANCCTGGNGTNTATCCNCCNCANGCAGCNGGTCATTTACCATATGTACTNTATGACCCNACAGCTACAGATGCAAANCAACACCAC AGNCANGGATANGAAAAGCCTGAAGAATTGTGGACNGCCAAAAGCCGTGTGCACCC ATTGTAAACANTCCCCACCGTGNCCTCAGCCAGGANNCGTNACNNANCGNCCNCCNG TNCCNCCCAGANTNTANNTGCCCCCNCCNNTACCNNNNAGNNANCCNNNNNNAAAA GATANANANNNTGTAGAATNTAANTNNNTAACNNGNTATGAACAACATGTNATTAGA ATG, or a complement thereof, wherein the nucleic acid is not more than about 450 nucleotides in length;
wherein N is a nucleotide chosen from a nucleotide a corresponding position in at least one of a B19 genome, A6 genome, or V9 genome as shown in FIG. 1.

29. The isolated nucleic acid of claim 28, wherein the nucleic acid is detectably labeled.


SEQUENCE LISTING (168 sequences)

SEQ ID NO: 1 - DNA, 5594 bp, human erythrovirus B19
ccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcttgcttggggttgccttgacactaagacaagcggcgcgccgcttgtcttagtggcacgtcaaccccaagcgctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttggtgtcttcttttaaattttagcgggcttttttcccgccttatgcaaatgggcagccattttaagtgtttcactataattttattggtcagttttgtaacggttaaaatgggcggagcgtaggcggggactacagtatatatagcacggcactgccgcagctctttctttctgggctgctttttcctggactttcttgctgttttttgtgagctaactaacaggtatttatactacttgttaacatactaacatggagctatttagaggggtgcttcaagtttcttctaatgttctggactgtgctaacgataactggtggtgctctttactggatttagacacttctgactgggaaccactaactcatactaacagactaatggcaatatacttaagcagtgtggcttctaagcttgactttaccggggggccactagcggggtgcttgtacttttttcaagtagaatgtaacaaatttgaagaaggctatcatattcatgtggttattggggggccagggttaaaccccagaaacctcacagtgtgtgtagaggggttatttaataatgtactttatcaccttgtaactgaaaatgtaaagctaaaatttttgccaggaatgactacaaaaggcaaatactttagagatggagagcagtttatagaaaactatttaatgaaaaaaatacctttaaatgttgtatggtgtgttactaatattgatggatatatagatacctgtatttctgctacttttagaaggggagcttgccatgccaagaaaccccgcattaccacagccataaatgacactagtagtgatgctggggagtctagcggcacaggggcagaggttgtgccaattaatgggaagggaactaaggctagcataaagtttcaaactatggtaaactggttgtgtgaaaacagagtgtttacagaggataagtggaaactagttgactttaaccagtacactttactaagcagtagtcacagtggaagttttcaaattcaaagtgcactaaaactagcaatttataaagcaactaatttagtgcctacaagcacatttctattgcatacagactttgagcaggttatgtgtattaaagacaataaaattgttaaattgttactttgtcaaaactatgaccccctattagtggggcagcatgtgttaaagtggattgataaaaaatgtggcaagaaaaatacactgtggttttatgggccgccaagtacaggaaaaacaaacttggcaatggccattgctaaaagtgttccagtatatggcatggttaactggaataatgaaaactttccatttaatgatgtagcagggaaaagcttggtggtctgggatgaaggtattattaagtctacaattgtagaagctgcaaaagccattttaggcgggcaacccaccagggtagatcaaaaaatgcgtggaagtgtagctgtgcctggagtacctgtggttataaccagcaatggtgacattacttttgttgtaagcgggaacactacaacaactgtacatgctaaagccttaaaagagcgaatggtaaagttaaactttactgtaagatgcagccctgacatggggttactaacagaggctgatgtacaacagtggcttacatggtgtaatgcacaaagctgggaccactatgaaaactgggcaataaactacacttttgatttccctggaattaatgcagatgccctccacccagacctccaaaccaccccaattgtcacagacaccagtatcagcagcagtggtggtgaaagctctgaagaactcagtgaaagcagcttttttaacctcatcaccccaggcgcctggaacactgaaaccccgcgctctagtacgcccatccccgggaccagttcaggagaatcatttgtcggaagctcagtttcctccgaagttgtagctgcatcgtgggaagaagccttctacacacctttggcagaccagtttcgtgaactgttagttggggttgattatgtgtgggacggtgtaaggggtttacctgtgtgttgtgtgcaacatattaacaatagtgggggaggcttgggactttgtccccattgcattaatgtaggggcttggtataatggatggaaatttcgagaatttaccccagatttggtgcggtgtagctgccatgtgggagcttctaatcccttttctgtgctaacctgcaaaaaatgtgcttacctgtctggattgcaaagctttgtagattatgagtaaagaaagtggcaaatggtgggaaagtgatgataaatttgctaaagctgtgtatcagcaatttgtggaattttatgaaaaggttactggaacagacttagagcttattcaaatattaaaagatcactataatatttctttagataatcccctagaaaacccatcctctctgtttgacttagttgctcgtattaaaaataaccttaaaaactctccagacttatatagtcatcattttcaaagtcatggacagttatctgaccacccccatgccttatcatccagtagcagtcatgcagaacctagaggagaaaatgcagtattatctagtgaagacttacacaagcctgggcaagttagcgtacaactacccggtactaactatgttgggcctggcaatgagctacaagctgggcccccgcaaagtgctgttgacagtgctgcaaggattcatgactttaggtatagccaactggctaagttgggaataaatccatatactcattggactgtagcagatgaagagcttttaaaaaatataaaaaatgaaactgggtttcaagcacaagtagtaaaagactactttactttaaaaggtgcagctgcccctgtggcccattttcaaggaagtttgccggaagttcccgcttacaacgcctcagaaaaatacccaagcatgacttcagttaattctgcagaagccagcactggtgcaggagggggtggcagtaatcctgtcaaaagcatgtggagtgagggggccacttttagtgccaactctgtaacttgtacattttccagacagtttttaattccttatgacccagagcaccattataaggtgttttctcccgcagcaagcagctgccacaatgccagtggaaaggaggcaaaggtttgcacaattagtcccataatgggatactcaaccccatggagatatttagattttaatgctttaaatttatttttttcacctttagagtttcagcacttaattgaaaattatggaagtatagctcctgatgctttaactgtaaccatatcagaaattgctgttaaggatgttacagacaaaactggagggggggtacaggttactgacagcactacagggcgcctatccatgttagtagaccatgaatacaagtacccatatgtgttaggacaaggtcaggatactttagccccagaacttcctatttgggtatactttccccctcaatatgcttacttaacagtaggagatgttaacacacaaggaatctctggagacagcaaaaaattagcaagtgaagaatcagcattttatgttttggaacacagttcttttcagcttttaggtacaggaggtacagcaactatgtcttataagtttcctccagtgcccccagaaaatttagagggctgcagtcaacacttttatgaaatgtacaatcccttatacggatcccgcttaggggttcctgacacattaggaggtgacccaaaatttagatctttaacacatgaagaccatgcaattcagccccaaaacttcatgccagggccactagtaaactcagtgtctacaaaggagggagacagctctaatactggagctggaaaagccttaacaggccttagcacaggcacctctcaaaacactagaatatccttacgccctgggccagtgtcacagccataccaccactgggacacagataaatatgttccaggaataaatgccatttctcatggtcagaccacttatggtaacgctgaagacaaagagtatcagcaaggagtgggtagatttccaaatgaaaaagaacagctaaaacagttacagggtttaaacatgcacacctatttccccaataaaggaacccagcaatatacagatcaaattgagcgccccctaatggtgggttctgtatggaacagaagagcccttcactatgaaagccagctgtggagtaaaattccaaatttagatgacagttttaaaactcagtttgcagccttaggaggatggggtttgcatcagccacctcctcaaatatttttaaaaatattaccacaaagtgggccaattggaggtattaaatcaatgggaattactaccttagttcagtatgccgtgggaattatgacagtaactatgacatttaaattggggccccgtaaagctacgggacggtggaatcctcaacctggagtatatcccccgcacgcagcaggtcatttaccatatgtactatatgaccccacagctacagatgcaaaacaacaccacaggcatggatacgaaaagcctgaagaattgtggacagccaaaagccgtgtgcacccattgtaaacactccccaccgtgccctcagccaggatgcgtaactaaacgcccaccagtaccacccagactgtacctgccccctcctgtacctataagacagcctaacacaaaagatatagacaatgtagaatttaagtacttaaccagatatgaacaacatgttattagaatgttaagattgtgtaatatgtatcaaaatttagaaaaataaacatttgttgtggttaaaaaattatgttgttgcgctttaaaaatttaaaagaagacaccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcgcttggggttgacgtgccactaagacaagcggcgcgccgcttgtcttagtgtcaaggcaaccccaagcaagctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttgg

SEQ ID NO: 2 - DNA, 4844 bp, human erythrovirus A6
ttttaaattttcgcgggctttttcccgccttatgcaaattacctgccatgtttaatgctttattttaatttgattggacaaaaagttaacggtttttatgggcggagttacgcaaggtatataagcagatgaattttgtaaaactttctttcctggctgctttttactggggataacttgctgttatttgcctgctaattaacaggtatttatactaacttttaatttactaacatggagctatttaggggtgtgttgcatatttcctctaacattttagactgtgctaatgataactggtggtgctctatgctggatttagatacttctgactgggaaccactaactcactctaacagactaatggcaatatatttaagtaatgttgcttctaaactggattttactggggggccgctggcgggttgcttatacttttttcaggtggaatgtaacaaatttgaggaaggctaccatattcatgtagttattggtggtccaggacttaatgctagaaacttaacagtgtgtgtagaaggcttgtttaataatgtgctttaccacctggtaaatgaaagtgttaaactgaaatttttgccaggaatgactacaaaaggaaagtattttagagatggagagcagtttatagaaaattacctaatgaaaaaaattcctttaaatgttgtgtggtgtgtaacaaatattgacgggtatatagacacctgtatttctgcatcttttagacgaggagcttgccatgctaaaaaacctcgaattagtacaaacacagacactgtaaataatgaaggaggggaatcaagctgtggagggggagatgtggtgccatttgccgggaagggaaccaaggcaggcttaaagtttcaaacaatggtaaattggctatgtgaaaacagagtgtttactgaagacaaatggaagttagtggattttaatcagtatacattattaagcagtagtcatagtgggagttttcaaatacaaagtgcattaaagctagctatttataaagctactaacttagttcctactagtacatttttaatgcattcagactttgagcaggttacctgcattaaagaaaataaaatagttaaactattattatgccagaattatgatcctcttttagtgggtcaacatgttttaaagtggattgacaaaaaatgtggtaaaaaaaacaccctgtggttttacgggcccccaagtactggaaaaacaaatttggcaatggctattgctaaaactgtcccagtgtatggcatggttaattggaataatgaaaattttccatttaatgatgtagcggggaaaagtttggtggtctgggatgaaggcattattaagtccactattgtggaagctgcaaaagccattttaggtgggcagccaaccagggtagatcaaaaaatgcgtggcagtgtggcagtgcctggtgtgccagtggtaataaccagcaatggtgacattacctttgttgtaagtggtaataccactacaactgtccatgctaaagccttaaaggagcgaatggtaaagctaaactttaccgtaagatgcagccctgacatgggcttacttacagaggctgatgtacagcaatggctaacttggtgtaatgcacaaagctggaaccactatgaaaactgggcaataaactacacgtttgatttccctggaataaatgcagatgccctccacccagacctccaaaccgtccccattgtcgcagacaccagtgtcagcagcagtggtggtgaaagctctgaagaactcggtgaaagcagctttttcaacctcatcactccaggcgcctggaacagtgaaaccccgcgctctagtacacccgtccccgggaccagttcaggagaatcatttgtcggaagcccagtttcctccgaagtggtagccgcgtcgtgggaggaagccttttacactccacttgcagaccagtttcgtgaactgttagttggggttgactatgtgtgggatggtgtgaggggattgcctgtttgttgtgtgcagcatattaataatagtgggggagggttaggcctttgtcctcattgtattaatgtgggagcttggtataatggatggaagtttcgtgaatttactccagatttggtacggtgcagctgccatgtaggagcttctaatcccttttctgtgttaacctgcaaaaaatgtgcttacttgtctggattacaaagttttgtggattatgagtaaaaaaagtgacaagtggtgggaaagtgatgataaatttgctaaggacgtgtataagcaatttgtagaattttatgaaaaagttactgagacagacttagagcttattcaaatattaaaagatcattataatatttctttagataatcccttagaaaacccatcttccctgtttgacttagttgctcgtattaaaagtaatcttaaagacactccagacctatatagtcatcattttcaaagtcatggacagttatttgaccacccccatgccttatcacccagtagcagtcatacagaacctagaggagaagatgcagtattatctagtgaagacttacacaagcctgggcaagttagcatacaactacccggtactaactatattggccctggcaatgagctacaagctgggcccccgcaaagtgctgtggacagtgctgcaaggattcatgactttaggtatagccaattggctaagctgggaataaatccatatactcattggactgtagcagatgaggaactgttaaaaaatataaaaaatgaaactgggtttcaagcacaagcagtaaaagattactttactttaaaaggtgcagctgcccctgtggcccattttcaaggaagtttgccggaagttcccgcatacaacgcctcagaaaagtacccaagcatgacttcagttaattctgcagaagccagcactggtgcaggagggggaggcagtaatcctgtcaaaagcatgtggagtgagggcgccacttttactgccaactctgtaacttgtacattttccagacagtttttaatcccatatgagccagagcaccgttataaagtgttttctcccgcagctagtagctgccataatgccagtgggaaagaggcaaaggtttgcactattagtcccataatgggctactcaacgccatggagatacttagactttaatgctttaaacttatttttttcacctttagaatttcaacatttaattgaaaattatggaagtatagcccctgatgctttaactgttaccatatcagaaattgctgttaaagatgttacagacaaaacaggaggaggggtgcaggttactgacagtactacagggcgtttatgcatgttagtagatcatgagtacaagtatccatatgtgttaggtcagggacaggataccttagccccagaactgcctatttgggtgtactttccccctcaatatgcttatttaaccgcgggagatgtaaacacacagggaatttcaggggacagtaaaaagctagcaagtgaagaatcagcattttatgttttggaacacagttcatttgaactgttaggtacaggtggctctgccactatgtcctataaatttccaccagtgcccccagaaaacttggagggttgtagccaacacttttatgaaatgtacaaccccctgtatgggtctcgtttaggggtacctgacacactagggggggaccctaaatttagatcattaactcacgaagatcatgcaattcagccacaaaactttatgcctggcccactagtaaactcagtgtccactaaagagggagacacttccaatacaggcgccggaaaagcccttacggggcttagtactggcactagtcaaagcaccagaatatccctgcgcccaggaccagtgtctcagccataccatcactgggacactgataagtatgtcacaggaataaatgctatttcacacggacagaccacttatggaaatgctgaagacaaagagtatcagcaaggggtaggaagattcccaaatgaaaaagagcaacttaaacagttacaaggcctaaacattcacacatactttccaaacaaaggaacccaacaatacacagatcaaattgaacgccccttaatggtagggtctgtgtggaacagaagagctcttcattatgagagtcagctgtggagtaaaatccccaacttagatgacagttttaaaacccaatttgcagccctgggcgggtggggtttacatcaaccacctcctcaaatatttttaaaaatactgccacagagtggaccaattgggggtattaaatccatgggaatcactaccctagttcaatatgcagtgggaattatgacagttactatgacatttaaattgggacctcgtaaggctactggtaggtggaatccacagcctggagtgtatcctcctcatgcagctggtcatttaccatatgtactgtatgaccctacagctacagatgcaaagcaacaccacagacacggatatgaaaagcctgaagaattgtggactgccaaaagccgtgtgcacccattgtaaacactccccaccgtgtcctcagccaggaaccgtaaccaaccgtcctcctgtaccacccagattatatgtgcccccgccaataccccgcagagaaccgcttgtaaaagatacaaatgctgtagaatataagttactaacccgttatgaacaacatgtaattagaatgcttagattgtgtaatatgtatacaaatttggaaaaataaataacttaaagaaatagctcatagtgtatgttactttaaaaatttttaaaa

SEQ ID NO: 3 - DNA, 5028 bp, human erythrovirus V9
gacgtcacaggaaatgacgtaactgtccgccatcttgtaccggaagtcccgcctaccggcggcgaccggcggcatctgatttggtgtcttctttttgaaattttggcgggctttttcccgccttatgcaaataagcggccatgtttaatgttatattttaatttaattggacaaacgcctaacggttactaggggcggagttacgggcggtatataagcagctgcgttccctgacactttcttttctggttgcttttgactggaactcacttgctgttctttgcctgctaagtaacaggtatttatactaacttttaatttactaacatggagctatttcggggtgtcttgcacatttcctctaacattctggactgtgctaatgataactggtggtgctctatgctagacttagatacttctgactgggaaccactaacccattctaacagattaatggcaatatatttaagcagtgttgcttctaaacttgattttactggggggccgctagcaggttgcttatacttttttcaggtggaatgtaacaaatttgaggaaggctatcatatccatgtagttattggtggtccaggactaaatgctagaaacttaactgtgtgcgtagaaggtttatttaataatgttctttaccatcttgtaactgaaagtgttaaacttaaatttttgccagggatgactaccaaaggaaaatattttagagatggagagcagtttatagaaaattacttaatgaaaaaaattcctttaaatgttgtgtggtgtgtaacaaatattgacgggtatatagacacctgtatttccgcctcttttcggcgaggagcttgtcatgctaaaagaccccgcattactgcaaatacagacagtgctactaatgaaactggggagtctagctgtggagggggagatgttgtgccattcgctggaaagggaacaaaagcggggttaaagtttcaaaccatggtaaattggctatgtgaaaacagagtatttactgaagataaatggaaattagtggattttaaccaatatactttattaagtagcagtcacagtggcagctttcaaattcaaagtgccttaaagttagctatttataaagctactaacttagtacccactagtacattcttgttacattcagactttgagcaggttacttgcattaaagaaaataaaatagtaaaattattattgtgtcaaaactatgatcctcttttagtgggtcaacatgtgttaaggtggattgacaaaaaatgtggtaaaaaaaacaccctgtggttttacgggccaccaagtactggaaaaacaaatttggcaatggctattgctaaaactgtaccagtgtatggaatggtgaattggaataatgaaaactttccatttaatgatgtagcggggaaaagtttggtggtctgggatgaaggcattattaagtccactattgtggaagctgcaaaagccattttaggtggtcagccaaccagggtagatcagaaaatgcgtggcagtgtggcagtgcccggtgtgcctgtggttataaccagcaatggtgacattacatttgttgtgagtggtaataccactacaactgtgcatgctaaagccttaaaggaacggatggtaaagctaaactttaccataagatgtagccctgacatgggtttacttacagaggctgatgtacaacaatggctaacttggtgtaatgcacaaagctggagccactatgaaaactgggcaataaactacacatttgatttccctggaataaatgcagatgccctccacccagatctccaaaccacccccattgtcccagacaccagtatcagcagcagtggtggtgaaagctctgaagaactcagtgaaagcagctttttcaacctcatcactccaggcgcctggaacagtgaaaccccgcgctctagtacgcccgtccccgggaccagttcaggagaatcatttgtcggaagcccagtttcctccgaagtggtagccgcgtcgtgggaggaagctttttacacgccgcttgccgatcagtttcgtgaactgttagtaggggttgactttgtatgggatggtgtgaggggattgcctgtttgctgtgtggaacatataaacaacagtgggggagggttggggctttgccctcattgtattaatgtgggagcttggtataatggatggaaatttagagagtttactccagacttagtgcgctgcagttgtcatgtaggagcctctaacccattttctgtgttaacttgtaaaaaatgtgcttacctgtctggattacaaagttttgtagattatgagtaaaaccactaacaaatggtgggaaagcagtgacaaatttgcccaggacgtgtataagcagtttgtgcaattttatgaaaaagctactggaacagacttagagcttattcaaattttaaaagaccattacaacatttctttagataatcctttagaaaacccctcttctttatttgacttagttgctcgcattaaaagtaatcttaaaaactctccagacctatatagtcatcattttcagagccatggacagttatctgaccacccccatgccttatcatccagtaacagtagtgcagaacctagaggagaaaatgcagtattatctagtgaagacttacacaagcctgggcaagttagcatacaattacccggtactaactatgttgggcctggcaatgagctacaagctgggcctccgcagaatgctgtggacagtgctgcaaggattcatgactttaggtatagccaattggctaagttgggaataaatccttatacacattggacggtagcagatgaagaattgttaaaaaatataaaaaatgaaacagggtttcaagcacaagcagtaaaagattactttactttaaaaggtgcagctgcccctgtggcccattttcaaggaagtttaccggaagtgcccgcgtacaacgcctcagaaaaataccccagcatgacttcagttaactctgcagaagccagcactggtgcaggcgggggaggtagcaaccctacaaaaagcatgtggagtgaaggggctacatttactgctaattctgtaacgtgtacattctctaggcaatttttaattccatatgatccagagcatcattataaagtgttctctccagcagctagtagctgccacaatgctagtgggaaagaggcaaaagtgtgcactattagtcccattatggggtactctactccgtggagatacttagattttaatgctttaaatttgtttttctcaccattagagtttcagcacttaattgaaaattatggtagtatagctccagatgctttaactgtaactatttcagaaattgctgtaaaagatgtcacagacaaaacaggaggaggtgtgcaagttactgacagcaccacaggacgtttgtgtatgttagtggatcatgagtataaatacccatatgtgctaggtcagggacaagacacactagctccagaactgcccatttgggtttactttcccccccagtatgcttacttaacagtaggtgaagtaaacacacaaggaatttcaggagacagcaaaaaattggctagtgaagaatcagctttttatgtgttagagcacagttcatttgaacttttgggtacagggggatctgccactatgtcctacaaatttccagctgtgcccccagaaaacctagaaggctgcagccaacatttttatgaaatgtacaaccctttgtacggttctcgtttaggggtacctgacacattaggaggggaccctaaatttagatcattgacacacgaagaccacgcaattcagccacaaaactttatgcctgggccactaataaattcagtgtctaccaaagaaggagacaattctaatacaggtgctggaaaagcccttacggggcttagtactggcactagccaaaacaccagaatttccctacgccccgggccagtatctcagccataccatcactgggacactgataaatatgttacaggaataaatgccatttcacatggacaaaccacttatggaaatgctgaggacaaagagtatcagcaaggggtaggaagatttccaaatgaaaaagaacagcttaagcagttacaaggtcttaacatgcacacatacttccctaataaaggaacccaacaatacacagaccaaattgaacgccctcttatggtgggctctgtttggaacagaagagctcttcactatgaaagtcagctgtggagtaaaatccctaacttagatgacagttttaaaactcaatttgcagccctaggcgggtggggtttgcatcaaccaccccctcaaatatttttaaaaatactaccacaaagtgggccaattggaggtattaaatccatgggaattactactttagttcaatatgctgtgggaataatgacagttaccatgacctttaaattgggacctcgaaaggctactggaaggtggaatccccagcctggcgtttatcctcctcatgcagctggtcatttaccatatgtactgtatgaccccacagctacagatgcaaagcaacaccacagacacggatatgaaaagcctgaagaattgtggactgccaaaagccgtgtgcacccattgtaaacattccccaccgtgtcctcagccaggaaccgtcacccaccgcccacctgtgccgcccagattatatgtgccccctccaataccccgtaggcaaccatctataaaagatacagacgctgtagaatataaattattaactagatatgaacaacatgtaattagaatgctaagattatgtaatatgtacacaagtttggaaaaataaaagccttaaataaataattcatagtgtatggttctttaaaaatttcaaaaagaagacaccaaatcagatgccgccggtcgccgccggtaggcgggacttccggtacaagatggcggacagttacgtcatttcctgtgacgtc

SEQ ID NO: 4 - DNA, 36 bp, human erythrovirus
  misc_feature (8)...(8): n = A, C or G
aaattttngcgggctttttncccgccttatgcaaat

SEQ ID NO: 5 - DNA, 274 bp, human erythrovirus
  misc_feature (19)...(19): n = C or A
taacaggtatttatactanntnttaanatactaacatggagctatttngnggngtnntncanntttcntctaannttntngactgtgctaangataactggtggtgctctntnctnganttaganacttctgactgggaaccactaacncannctaacagantaatggcaatatanttaagnantgtngcttctaanctngantttacnggggggccnctngcnggntgcttntacttttttcangtngaatgtaacaaatttgangaaggcta

SEQ ID NO: 6 - DNA, 143 bp, human erythrovirus
  misc_feature (15)...(15): n = A or G
aaatttttgccaggnatgactacnaaaggnaantantttagagatggagagcagtttatagaaaantanntaatgaaaaaaatncctttaaatgtngtatggtgtgtnacnaatattganggntatataganacctgtatttc

SEQ ID NO: 7 - DNA, 316 bp, human erythrovirus
  misc_feature (14)...(14): n = T, A or C
taaagtttcaaacnatggtaaantggntntgtgaaaacagagtntttacnganganaantggaanntagtngantttaancantanacnttantaagnagnagtcanagtggnagntttcaaatncaaagtgcnntaaanntagcnatttataaagcnactaanttagtnccnacnagnacattnntnntncatncagactttgagcaggttanntgnattaaaganaataaaatngtnaaantnttantntgncanaantatganccnctnttagtgggncancatgtnttaangtggattganaaaaaatgtgg

SEQ ID NO: 8 - DNA, 776 bp, human erythrovirus
  misc_feature (9)...(9): n = A or T
ccaagtacnggaaaaacaaanttggcaatggcnattgctaaaantgtnccagtntatggnatggtnaantggaataatgaaaantttccatttaatgatgtagcngggaaaagnttggtggtctgggatgaaggnattattaagtcnacnattgtngaagctgcaaaagccattttaggnggncanccnaccagggtagatcanaaaatgcgtggnagtgtngcngtgccnggngtnccngtggtnataaccagcaatggtgacattacntttgttgtnagnggnaanacnacnacaactgtncatgctaaagccttaaangancgnatggtaaagntaaactttacnntaagatgnagccctgacatgggnttactnacagaggctgatgtacancantggctnacntggtgtaatgcacaaagctggnnccactatgaaaactgggcaataaactacacntttgatttccctggaatnaatgcagatgccctccacccagacctncaaaccnncccnattgtcncagacaccagtntcagcagcagtggtggtgaaagctctgaagaactcngtgaaagcagctttttnaacctcatcacnccaggcgcctggaacantgaaaccccgcgctctagtacncccntccccgggaccagttcaggagaatcatttgtcggaagcncagtttcctccgaagtngtagcngcntcgtgggangaagcnttntacacnccnntngcngancagtttcgtgaactgttagtnggggttga

SEQ ID NO: 9 - DNA, 257 bp, human erythrovirus
  misc_feature (21)...(21): n = T or A
cagtttcgtgaactgttagtnggggttgantatgtntggganggtgtnaggggnttncctgtntgntgtgtgnancatatnaanaanagtgggggaggnttnggnctttgnccncattgnattaatgtnggngcttggtataatggatggaantttngngantttacnccaganttngtncgntgnagntgncatgtnggagcntctaanccnttttctgtgctaacntgnaaaaaatgtgcttacntgtctggatt

SEQ ID NO: 10 - DNA, 751 bp, human erythrovirus
  misc_feature (16)...(16): n = C or T
aaaaaatgtgcttacntgtctggattncaaagntttgtngattatgagtaaannnantnncaantggtgggaaagnnntganaaatttgcnnangnngtgtatnagcantttgtnnaattttatgaaaangntactgnnacagacttagagcttattcaaatnttaaaagancantanaanatttctttagataatccnntagaaaacccntcntcnntntttgacttagttgctcgnattaaaantaancttaaanacnctccagacntatatagtcatcattttcanagncatggacagttatntgaccacccccatgccttatcanccagtancagtnntncagaacctagaggagaanatgcagtattatctagtgaagacttacacaagcctgggcaagttagcntacaantacccggtactaactatnttggncctggcaatgagctacaagctgggccnccgcanantgctgtngacagtgctgcaaggattcatgactttaggtatagccaantggctaagntgggaataaatccntatacncattggacngtagcagatganganntnttaaaaaatataaaaaatgaaacngggtttcaagcacaagnagtaaaagantactttactttaaaaggtgcagctgcccctgtggcccattttcaaggaagtttnccggaagtncccgcntacaacgcctcagaaaantacccnagcatgacttcagttaantctgcagaagcc

SEQ ID NO: 11 - DNA, 450 bp, human erythrovirus
  misc_feature (2)...(2): n = A or G
tnccacanagtggnccaattggnggtattaaatcnatgggaatnactacnntagttcagtatgcngtgggaattatgacagtnacnatgacntttaaattgggnccncgnaangctacnggnnggtggaatccncancctggngtntatccnccncangcagcnggtcatttaccatatgtactntatgacccnacagctacagatgcaaancaacaccacagncanggatangaaaagcctgaagaattgtggacngccaaaagccgtgtgcacccattgtaaacantccccaccgtgncctcagccagganncgtnacnnancgnccnccngtnccncccagantntanntgcccccnccnntaccnnnnagnnanccnnnnnnaaaagatananannntgtagaatntaantnnntaacnngntatgaacaacatgtnattagaatg

SEQ ID NO: 12 - DNA, 19 bp, Artificial Sequence
  oligonucleotide
aaattttagcgggcttttt

SEQ ID NO: 13 - DNA, 16 bp, Artificial Sequence
  oligonucleotide
cccgccttatgcaaat

SEQ ID NO: 14 - DNA, 18 bp, Artificial Sequence
  oligonucleotide
taacaggtatttatacta

SEQ ID NO: 15 - DNA, 19 bp, Artificial Sequence
  oligonucleotide
tactaacatggagctattt

SEQ ID NO: 16 - DNA, 30 bp, Artificial Sequence
  oligonucleotide
gactgtgctaacgataactggtggtgctct

SEQ ID NO: 17 - DNA, 23 bp, Artificial Sequence
  oligonucleotide
acttctgactgggaaccactaac

SEQ ID NO: 18 - DNA, 22 bp, Artificial Sequence
  oligonucleotide
ctaacagactaatggcaatata

SEQ ID NO: 19 - DNA, 26 bp, Artificial Sequence
  oligonucleotide
gaatgtaacaaatttgaagaaggcta

SEQ ID NO: 20 - DNA, 23 bp, Artificial Sequence
  oligonucleotide
aaatttttgccaggaatgactac

SEQ ID NO: 21 - DNA, 29 bp, Artificial Sequence
  oligonucleotide
tttagagatggagagcagtttatagaaaa

SEQ ID NO: 22 - DNA, 37 bp, Artificial Sequence
  oligonucleotide
taatgaaaaaaatacctttaaatgttgtatggtgtgt

SEQ ID NO: 23 - DNA, 20 bp, Artificial Sequence
  oligonucleotide
tatatagatacctgtatttc

SEQ ID NO: 24 - DNA, 22 bp, Artificial Sequence
  oligonucleotide
taaagtttcaaactatggtaaa

SEQ ID NO: 25 - DNA, 17 bp, Artificial Sequence
  oligonucleotide
tttcaaattcaaagtgc

SEQ ID NO: 26 - DNA, 18 bp, Artificial Sequence
  oligonucleotide
cagactttgagcaggtta

SEQ ID NO: 27 - DNA, 17 bp, Artificial Sequence
  oligonucleotide
attaaagacaataaaat

SEQ ID NO: 28 - DNA, 21 bp, Artificial Sequence
  oligonucleotide
gtggattgataaaaaatgtgg

SEQ ID NO: 29 - DNA, 43 bp, Artificial Sequence
  oligonucleotide
ccaagtacaggaaaaacaaacttggcaatggccattgctaaaa

SEQ ID NO: 30 - DNA, 77 bp, Artificial Sequence
  oligonucleotide
tggaataatgaaaactttccatttaatgatgtagcagggaaaagcttggtggtctgggatgaaggtattattaagtc

SEQ ID NO: 31 - DNA, 35 bp, Artificial Sequence
  oligonucleotide
tggaataatgaaaactttccatttaatgatgtagc

SEQ ID NO: 32 - DNA, 16 bp, Artificial Sequence
  oligonucleotide
tggtggtctgggatga

SEQ ID NO: 33 - DNA, 22 bp, Artificial Sequence
  oligonucleotide
ttcgacgttttcggtaaaatcc

SEQ ID NO: 34 - DNA, 23 bp, Artificial Sequence
  oligonucleotide
gaagctgcaaaagccattttagg

SEQ ID NO: 35 - DNA, 26 bp, Artificial Sequence
  oligonucleotide
accagggtagatcaaaaaatgcgtgg

SEQ ID NO: 36 - DNA, 32 bp, Artificial Sequence
  oligonucleotide
ataaccagcaatggtgacattacttttgttgt

SEQ ID NO: 37 - DNA, 26 bp, Artificial Sequence
  oligonucleotide
acaactgtacatgctaaagccttaaa

SEQ ID NO: 38 - DNA, 26 bp, Artificial Sequence
  oligonucleotide
acaactgtacatgctaaagccttaaa

SEQ ID NO: 39 - DNA, 20 bp, Artificial Sequence
  oligonucleotide
atggtaaagttaaactttac

SEQ ID NO: 40 - DNA, 22 bp, Artificial Sequence
  oligonucleotide
taagatgcagccctgacatggg

SEQ ID NO: 41 - DNA, 14 bp, Artificial Sequence
  oligonucleotide
agccctgacatggg

SEQ ID NO: 42 - DNA, 17 bp, Artificial Sequence
  oligonucleotide
acagaggctgatgtaca

SEQ ID NO: 43 - DNA, 21 bp, Artificial Sequence
  oligonucleotide
tggtgtaatgcacaaagctgg

SEQ ID NO: 44 - DNA, 19 bp, Artificial Sequence
  oligonucleotide
cacattacgtgtttcgacc

SEQ ID NO: 45 - DNA, 19 bp, Artificial Sequence
  oligonucleotide
gtgtaatgcacaaagctgg

SEQ ID NO: 46 - DNA, 82 bp, Artificial Sequence
  oligonucleotide
ccactatgaaaactgggcaataaactacacttttgatttccctggaattaatgcagatgccctccacccagacctccaaacc

SEQ ID NO: 47 - DNA, 122 bp, Artificial Sequence
  oligonucleotide
attgtcacagacaccagtatcagcagcagtggtggtgaaagctctgaagaactcagtgaaagcagcttttttaacctcatcaccccaggcgcctggaacactgaaaccccgcgctctagtac

SEQ ID NO: 48 - DNA, 55 bp, Artificial Sequence
  oligonucleotide
tccccgggaccagttcaggagaatcatttgtcggaagctcagtttcctccgaagt

SEQ ID NO: 49 - DNA, 22 bp, Artificial Sequence
  oligonucleotide
gaagctcagtttcctccgaagt

SEQ ID NO: 50 - DNA, 29 bp, Artificial Sequence
  oligonucleotide
cagtttcgtgaactgttagttggggttga

SEQ ID NO: 51 - DNA, 20 bp, Artificial Sequence
  oligonucleotide
ttctacacacctttggcaga

SEQ ID NO: 52 - DNA, 24 bp, Artificial Sequence
  oligonucleotide
caaagcacttgacaatcaacccca

SEQ ID NO: 53 - DNA, 24 bp, Artificial Sequence
  oligonucleotide
gtttcgtgaactgttagttggggt

SEQ ID NO: 54 - DNA, 43 bp, Artificial Sequence
  oligonucleotide
tgtaggagcttggtataatggatggaaatttcgagaatttacc

SEQ ID NO: 55 - DNA, 52 bp, Artificial Sequence
  oligonucleotide
aaaaaatgtgcttacctgtctggattacaaagctttgtagattatgagtaaa

SEQ ID NO: 56 - DNA, 41 bp, Artificial Sequence
  oligonucleotide
ccacaaagtgggccaattggaggtattaaatcaatgggaat

SEQ ID NO: 57 - DNA, 17 bp, Artificial Sequence
  oligonucleotide
gtgggaattatgacagt

SEQ ID NO: 58 - DNA, 59 bp, Artificial Sequence
  oligonucleotide
ggtcatttaccatatgtactatatgaccccacagctacagatgcaaaacaacaccacag

SEQ ID NO: 59 - DNA, 79 bp, Artificial Sequence
  oligonucleotide
gaaaagcctgaagaattgtggacagccaaaagccgtgtgcacccattgtaaacactccccaccgtgccctcagccagga

SEQ ID NO: 60 - DNA, 24 bp, Artificial Sequence
  oligonucleotide
tatgaacaacatgttattagaatg

SEQ ID NO: 61 - DNA, 19 bp, Artificial Sequence
  oligonucleotide
aaattttcgcgggcttttt

SEQ ID NO: 62 - DNA, 16 bp, Artificial Sequence
  oligonucleotide
cccgccttatgcaaat

SEQ ID NO: 63 - DNA, 18 bp, Artificial Sequence
  oligonucleotide
taacaggtatttatacta

SEQ ID NO: 64 - DNA, 19 bp, Artificial Sequence
  oligonucleotide
tactaacatggagctattt

SEQ ID NO: 65 - DNA, 30 bp, Artificial Sequence
  oligonucleotide
gactgtgctaatgataactggtggtgctct

SEQ ID NO: 66 - DNA, 23 bp, Artificial Sequence
  oligonucleotide
acttctgactgggaaccactaac

SEQ ID NO: 67 - DNA, 22 bp, Artificial Sequence
  oligonucleotide
ctaacagactaatggcaatata

SEQ ID NO: 68 - DNA, 26 bp, Artificial Sequence
  oligonucleotide
gaatgtaacaaatttgaggaaggcta

SEQ ID NO: 69 - DNA, 23 bp, Artificial Sequence
  oligonucleotide
aaatttttgccaggaatgactac

SEQ ID NO: 70 - DNA, 29 bp, Artificial Sequence
  oligonucleotide
tttagagatggagagcagtttatagaaaa

SEQ ID NO: 71 - DNA, 37 bp, Artificial Sequence
  oligonucleotide
taatgaaaaaaattcctttaaatgttgtgtggtgtgt

SEQ ID NO: 72 - DNA, 20 bp, Artificial Sequence
  oligonucleotide
tatatagacacctgtatttc

SEQ ID NO: 73 - DNA, 22 bp, Artificial Sequence
  oligonucleotide
taaagtttcaaacaatggtaaa

SEQ ID NO: 74 - DNA, 17 bp, Artificial Sequence
  oligonucleotide
tttcaaatacaaagtgc

SEQ ID NO: 75 - DNA, 18 bp, Artificial Sequence
  oligonucleotide
cagactttgagcaggtta

SEQ ID NO: 76 - DNA, 17 bp, Artificial Sequence
  oligonucleotide
attaaagaaaataaaat

SEQ ID NO: 77 - DNA, 21 bp, Artificial Sequence
  oligonucleotide
gtggattgacaaaaaatgtgg

SEQ ID NO: 78 - DNA, 43 bp, Artificial Sequence
  oligonucleotide
ccaagtactggaaaaacaaacttggcaatggctattgctaaaa

SEQ ID NO: 79 - DNA, 77 bp, Artificial Sequence
  oligonucleotide
tggaataatgaaaattttccatttaatgatgtagcggggaaaagtttggtggtctgggatgaaggcattattaagtc

SEQ ID NO: 80 - DNA, 23 bp, Artificial Sequence
  oligonucleotide
gaagctgcaaaagccattttagg

SEQ ID NO: 81 - DNA, 26 bp, Artificial Sequence
  oligonucleotide
accagggtagatcaaaaaatgcgtgg

SEQ ID NO: 82 - DNA, 32 bp, Artificial Sequence
  oligonucleotide
ataaccagcaatggtgacattacctttgttgt

SEQ ID NO: 83 - DNA, 26 bp, Artificial Sequence
  oligonucleotide
acaactgtccatgctaaagccttaaa

SEQ ID NO: 84 - DNA, 20 bp, Artificial Sequence
  oligonucleotide
atggtaaagctaaactttac

SEQ ID NO: 85 - DNA, 22 bp, Artificial Sequence
  oligonucleotide
taagatgcagccctgacatggg

SEQ ID NO: 86 - DNA, 17 bp, Artificial Sequence
  oligonucleotide
acagaggctgatgtaca

SEQ ID NO: 87 - DNA, 21 bp, Artificial Sequence
  oligonucleotide
tggtgtaatgcacaaagctgg

SEQ ID NO: 88 - DNA, 82 bp, Artificial Sequence
  oligonucleotide
ccactatgaaaactgggcaataaactacacgtttgatttccctggaataaatgcagatgccctccacccagacctccaaacc

SEQ ID NO: 89 - DNA, 122 bp, Artificial Sequence
  oligonucleotide
attgtcgcagacaccagtgtcagcagcagtggtggtgaaagctctgaagaactcggtgaaagcagctttttcaacctcatcactccaggcgcctggaacagtgaaaccccgcgctctagtac

SEQ ID NO: 90 - DNA, 20 bp, Artificial Sequence
  oligonucleotide
tttacactccacttgcagac

SEQ ID NO: 91 - DNA, 55 bp, Artificial Sequence
  oligonucleotide
tccccgggaccagttcaggagaatcatttgtcggaagcccagtttcctccgaagt

SEQ ID NO: 92 - DNA, 29 bp, Artificial Sequence
  oligonucleotide
cagtttcgtgaactgttagttggggttga

SEQ ID NO: 93 - DNA, 43 bp, Artificial Sequence
  oligonucleotide
tgtgggagcttggtataatggatggaagtttcgtgaatttact

SEQ ID NO: 94 - DNA, 52 bp, Artificial Sequence
  oligonucleotide
aaaaaatgtgcttacttgtctggattacaaagttttgtggattatgagtaaa

SEQ ID NO: 95 - DNA, 39 bp, Artificial Sequence
  oligonucleotide
tactgagacagacttagagcttattcaaatattaaaaga

SEQ ID NO: 96 - DNA, 23 bp, Artificial Sequence
  oligonucleotide
atttctttagataatcccttaga

SEQ ID NO: 97 - DNA, 25 bp, Artificial Sequence
  oligonucleotide
tttgacttagttgctcgtattaaaa

SEQ ID NO: 98 - DNA, 28 bp, Artificial Sequence
  oligonucleotide
ctccagacctatatagtcatcattttca

SEQ ID NO: 99 - DNA, 43 bp, Artificial Sequence
  oligonucleotide
catggacagttatttgaccacccccatgccttatcacccagta

SEQ ID NO: 100 - DNA, 65 bp, Artificial Sequence
  oligonucleotide
cagaacctagaggagaagatgcagtattatctagtgaagacttacacaagcctgggcaagttagc

SEQ ID NO: 101 - DNA, 49 bp, Artificial Sequence
  oligonucleotide
tacccggtactaactatattggccctggcaatgagctacaagctgggcc

SEQ ID NO: 102 - DNA, 69 bp, Artificial Sequence
  oligonucleotide
tgctgtggacagtgctgcaaggattcatgactttaggtatagccaattggctaagctgggaataaatcc

SEQ ID NO: 103 - DNA, 20 bp, Artificial Sequence
  oligonucleotide
cattggactgtagcagatga

SEQ ID NO: 104 - DNA, 113 bp, Artificial Sequence
  oligonucleotide
ttaaaaaatataaaaaatgaaactgggtttcaagcacaagcagtaaaagattactttactttaaaaggtgcagctgcccctgtggcccattttcaaggaagtttgccggaagt

SEQ ID NO: 105 - DNA, 17 bp, Artificial Sequence
  oligonucleotide
tacaacgcctcagaaaa

SEQ ID NO: 106 - DNA, 44 bp, Artificial Sequence
  oligonucleotide
agcatgacttcagttaattctgcagaagccagcactggtgcagg

SEQ ID NO: 107 - DNA, 41 bp, Artificial Sequence
  oligonucleotide
ccacagagtggaccaattgggggtattaaatccatgggaat

SEQ ID NO: 108 - DNA, 17 bp, Artificial Sequence
  oligonucleotide
gtgggaattatgacagt

SEQ ID NO: 109 - DNA, 59 bp, Artificial Sequence
  oligonucleotide
ggtcatttaccatatgtactgtatgaccctacagctacagatgcaaagcaacaccacag

SEQ ID NO: 110 - DNA, 79 bp, Artificial Sequence
  oligonucleotide
gaaaagcctgaagaattgtggactgccaaaagccgtgtgcacccattgtaaacactccccaccgtgtcctcagccagga

SEQ ID NO: 111 - DNA, 24 bp, Artificial Sequence
  oligonucleotide
tatgaacaacatgtaattagaatg

SEQ ID NO: 112 - DNA, 19 bp, Artificial Sequence
  oligonucleotide
aaattttggcgggcttttt

SEQ ID NO: 113 - DNA, 16 bp, Artificial Sequence
  oligonucleotide
cccgccttatgcaaat

SEQ ID NO: 114 - DNA, 18 bp, Artificial Sequence
  oligonucleotide
taacaggtatttatacta

SEQ ID NO: 115 - DNA, 19 bp, Artificial Sequence
  oligonucleotide
tactaacatggagctattt

SEQ ID NO: 116 - DNA, 30 bp, Artificial Sequence; identical to SEQ ID NO: 65
  oligonucleotide

SEQ ID NO: 117 - DNA, 23 bp, Artificial Sequence
  oligonucleotide
acttctgactgggaaccactaac

SEQ ID NO: 118 - DNA, 22 bp, Artificial Sequence
  oligonucleotide
ctaacagattaatggcaatata

SEQ ID NO: 119 - DNA, 26 bp, Artificial Sequence
  oligonucleotide
gaatgtaacaaatttgaggaaggcta

SEQ ID NO: 120 - DNA, 23 bp, Artificial Sequence
  oligonucleotide
aaatttttgccagggatgactac

SEQ ID NO: 121 - DNA, 29 bp, Artificial Sequence
  oligonucleotide
tttagagatggagagcagtttatagaaaa

SEQ ID NO: 122 - DNA, 37 bp, Artificial Sequence; identical to SEQ ID NO: 71
  oligonucleotide

SEQ ID NO: 123 - DNA, 20 bp, Artificial Sequence
  oligonucleotide
tatatagacacctgtatttc

SEQ ID NO: 124 - DNA, 22 bp, Artificial Sequence
  oligonucleotide
taaagtttcaaaccatggtaaa

SEQ ID NO: 125 - DNA, 17 bp, Artificial Sequence
  oligonucleotide
tttcaaattcaaagtgc

SEQ ID NO: 126 - DNA, 18 bp, Artificial Sequence
  oligonucleotide
cagactttgagcaggtta

SEQ ID NO: 127 - DNA, 17 bp, Artificial Sequence
  oligonucleotide
attaaagaaaataaaat

SEQ ID NO: 128 - DNA, 21 bp, Artificial Sequence
  oligonucleotide
gtggattgacaaaaaatgtgg

SEQ ID NO: 129 - DNA, 43 bp, Artificial Sequence; identical to SEQ ID NO: 78
  oligonucleotide

SEQ ID NO: 130 - DNA, 77 bp, Artificial Sequence
  oligonucleotide
tggaataatgaaaactttccatttaatgatgtagcggggaaaagtttggtggtctgggatgaaggcattattaagtc

SEQ ID NO: 131 - DNA, 23 bp, Artificial Sequence
  oligonucleotide
gaagctgcaaaagccattttagg

SEQ ID NO: 132 - DNA, 26 bp, Artificial Sequence
  oligonucleotide
accagggtagatcagaaaatgcgtgg

SEQ ID NO: 133 - DNA, 32 bp, Artificial Sequence
  oligonucleotide
ataaccagcaatggtgacattacatttgttgt

SEQ ID NO: 134 - DNA, 26 bp, Artificial Sequence
  oligonucleotide
acaactgtgcatgctaaagccttaaa

SEQ ID NO: 135 - DNA, 20 bp, Artificial Sequence
  oligonucleotide
atggtaaagctaaactttac

SEQ ID NO: 136 - DNA, 22 bp, Artificial Sequence
  oligonucleotide
taagatgtagccctgacatggg

SEQ ID NO: 137 - DNA, 17 bp, Artificial Sequence
  oligonucleotide
acagaggctgatgtaca

SEQ ID NO: 138 - DNA, 21 bp, Artificial Sequence
  oligonucleotide
tggtgtaatgcacaaagctgg

SEQ ID NO: 139 - DNA, 82 bp, Artificial Sequence
  oligonucleotide
ccactatgaaaactgggcaataaactacacatttgatttccctggaataaatgcagatgccctccacccagatctccaaacc

SEQ ID NO: 140 - DNA, 122 bp, Artificial Sequence
  oligonucleotide
attgtcccagacaccagtatcagcagcagtggtggtgaaagctctgaagaactcagtgaaagcagctttttcaacctcatcactccaggcgcctggaacagtgaaaccccgcgctctagtac

SEQ ID NO: 141 - DNA, 18 bp, Artificial Sequence
  oligonucleotide
atttgcccaggacgtgta

SEQ ID NO: 142 - DNA, 55 bp, Artificial Sequence; identical to SEQ ID NO: 91
  oligonucleotide

SEQ ID NO: 143 - DNA, 29 bp, Artificial Sequence
  oligonucleotide
cagtttcgtgaactgttagtaggggttga

SEQ ID NO: 144 - DNA, 44 bp, Artificial Sequence
  oligonucleotide
tgtgggagcttggtataatggatggaaaatttagagagtttact

SEQ ID NO: 145 - DNA, 52 bp, Artificial Sequence
  oligonucleotide
aaaaaatgtgcttacctgtctggattacaaagttttgtggattatgagtaaa

SEQ ID NO: 146 - DNA, 34 bp, Artificial Sequence
  oligonucleotide
gtctggattacaaagctttgtagattatgagtaa

SEQ ID NO: 147 - DNA, 32 bp, Artificial Sequence
  oligonucleotide
taaagaaatctattaggaaatctcttggggag

SEQ ID NO: 148 - DNA, 32 bp, Artificial Sequence
  oligonucleotide
atttctttagataatcccctagaaaacccatc

SEQ ID NO: 149 - DNA, 18 bp, Artificial Sequence
  oligonucleotide
atttgcccaggacgtgta

SEQ ID NO: 150 - DNA, 39 bp, Artificial Sequence
  oligonucleotide
tactggaacagacttagagcttattcaaattttaaaaga

SEQ ID NO: 151 - DNA, 23 bp, Artificial Sequence
  oligonucleotide
atttctttagataatcctttaga

SEQ ID NO: 152 - DNA, 25 bp, Artificial Sequence
  oligonucleotide
tttgacttagttgctcgcattaaaa

SEQ ID NO: 153 - DNA, 28 bp, Artificial Sequence
  oligonucleotide
ctccagacctatatagtcatcattttca

SEQ ID NO: 154 - DNA, 43 bp, Artificial Sequence
  oligonucleotide
catggacagttatctgaccacccccatgccttatcatccagta

SEQ ID NO: 155 - DNA, 65 bp, Artificial Sequence
  oligonucleotide
cagaacctagaggagaaaatgcagtattatctagtgaagacttacacaagcctgggcaagttagc

SEQ ID NO: 156 - DNA, 49 bp, Artificial Sequence
  oligonucleotide
tacccggtactaactatgttgggcctggcaatgagctacaagctgggcc

SEQ ID NO: 157 - DNA, 69 bp, Artificial Sequence
  oligonucleotide
tgctgtggacagtgctgcaaggattcatgactttaggtatagccaattggctaagttgggaataaatcc

SEQ ID NO: 158 - DNA, 20 bp, Artificial Sequence
  oligonucleotide
cattggacggtagcagatga

SEQ ID NO: 159 - DNA, 113 bp, Artificial Sequence
  oligonucleotide
ttaaaaaatataaaaaatgaaacagggtttcaagcacaagcagtaaaagattactttactttaaaaggtgcagctgcccctgtggcccattttcaaggaagtttaccggaagt

SEQ ID NO: 160 - DNA, 17 bp, Artificial Sequence
  oligonucleotide
tacaacgcctcagaaaa

SEQ ID NO: 161 - DNA, 44 bp, Artificial Sequence
  oligonucleotide
agcatgacttcagttaactctgcagaagccagcactggtgcagg

SEQ ID NO: 162 - DNA, 41 bp, Artificial Sequence
  oligonucleotide
ccacaaagtgggccaattggaggtattaaatccatgggaat

SEQ ID NO: 163 - DNA, 17 bp, Artificial Sequence
  oligonucleotide
gtgggaattatgacagt

SEQ ID NO: 164 - DNA, 59 bp, Artificial Sequence
  oligonucleotide
ggtcatttaccatatgtactgtatgaccccacagctacagatgcaaagcaacaccacag

SEQ ID NO: 165 - DNA, 79 bp, Artificial Sequence
  oligonucleotide
gaaaagcctgaagaattgtggactgccaaaagccgtgtgcacccattgtaaacattccccaccgtgtcctcagccagga

SEQ ID NO: 166 - DNA, 24 bp, Artificial Sequence
  oligonucleotide
tatgaacaacatgtaattagaatg

SEQ ID NO: 167 - DNA, 20 bp, Artificial Sequence
  oligonucleotide
acacaactgtgttcactagc

SEQ ID NO: 168 - DNA, 20 bp, Artificial Sequence
  oligonucleotide
caacttcatccacgttcacc